(19) United States
(12) Patent Application Publication
(10) Pub. No.: US 2019/0119350 A1
(43) Pub. Date: Apr. 25, 2019

(54) NY-ESO-1 SPECIFIC TCRS AND METHODS OF USE THEREOF

(71) Applicant: IMMUNE DESIGN CORP., Seattle, WA (US)

(72) Inventors: Hailing LU, Seattle, WA (US); Jan Henrik TER MEULEN, Seattle, WA (US)

(21) Appl. No.: 15/756,440

(22) PCT Filed: Sep. 8, 2016
(86) PCT No.: PCT/US16/50826

§ 371 Date: Feb. 28, 2018

Related U.S. Application Data
(60) Provisional application No. 62/216,099, filed on Sep. 9, 2015.
(60) Provisional application No. 62/377,276, filed on Aug. 19, 2016.

(51) Int. Cl. C07K 14/725; A61P 35/00; G01N 33/574; A61K 31/7016; A61K 9/00; A61K 45/06; A61K 38/17

(52) U.S. Cl. CPC C07K 14/7051; A61P 35/00; G01N 33/57492; A61K 31/7016; C07K 2319/035; A61K 45/06; A61K 38/1774; C07K 2319/03; A61K 9/0019

(57) ABSTRACT

The present disclosure relates to NY-ESO-1 specific TCR amino acid sequences and methods of their use.

### BACKGROUND

One approach to treating cancer patients is to genetically modify T cells to target antigens expressed on tumor cells through the expression of chimeric antigen receptors (CARs) or recombinant T-cell receptors (rTCRs) for adoptive cell therapy (ACT). CARs are antigen receptors that are designed to recognize cell surface antigens in a human leukocyte antigen-independent manner. Attempts in using genetically modified cells expressing CARs to treat certain types of cancers have met with impressive success, especially in CD19 expressing liquid tumors. See for example, Porter David L, Levine Bruce L, Kalos Michael, Bagg Adam, June Carl H: Chimeric antigen receptor-modified T cells in chronic lymphoid leukemia. The New England journal of medicine 365(8): 725-33, August 2011; Kalos Michael, Levine Bruce L, Porter David L, Katz Sharyn, Grupp Stephan A, Bagg Adam, June Carl H: T cells with chimeric antigen receptors have potent antitumor effects and can establish memory in patients with advanced Leukemia. Science translational medicine 3(95): 95ra73, August 2011. Likewise, autologous T cells engineered to express a recombinant T cell receptor specific to a MHC-restricted peptide derived from the cancer-testis antigen NY-ESO-1 have recently shown impressive clinical efficacy in multiple myeloma and sarcoma. See for example, Rapoport A P, Stadtmauer E A, Binder-Scholl G K, Goloubeva 0, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin H K, Ribeiro L, Holdich T, Yanovich S, Hardy N, Yared J, Kerr N, Philip S, Westphal S, Siegel D L, Levine B L, Jakobsen B K, Kalos M, June C H. NY-ESO-1-specific TCR-engineered T cells mediate sustained antigen-specific antitumor effects in myeloma. Nat Med. 2015 August; 21(8):914-21; Robbins P F, Kassim S H, Tran T L, Crystal J S, Morgan R A, Feldman S A, Yang J C, Dudley M E, Wunderlich J R, Sherry R M, Kammula U S, Hughes M S, Restifo N P, Raffeld M, Lee C C, Li Y F, El-Gamil M, Rosenberg S A. Pilot trial using lymphocytes genetically engineered with an NY-ESO-1-reactive T-cell receptor: long-term follow-up and correlates with response. Clin Cancer Res. 2015 Mar. 1; 21(5):1019-27.)

However, setbacks have also been encountered, namely toxicities, especially in the case of rTCR with in vitro enhanced affinities (see e.g., Linette G P, Stadtmauer E A, Maus M V, Rapoport A P, Levine B L, Emery L, Litzky L, Bagg A, Carreno B M, Cimino P J, Binder-Scholl GK, Smethurst D P, Gerry A B, Pumphrey N J, Bennett A D, Brewer J E, Dukes J, Harper J, Tayton-Martin H K, Jakobsen B K, Hassan N J, Kalos M, June C H. Cardiovascular toxicity and titin cross-reactivity of affinity-enhanced T cells in myeloma and melanoma. Blood. 2013 Aug. 8; 122(6):863-71.). Accordingly, notwithstanding the foregoing efforts, there remains a continuing need for more effective and safe methods for redirecting T cells to tumors or other targets of interest.

Several methods are currently being employed for selection of T cell receptors for adoptive cell therapy, including immunization of human HLA transgenic mice and isolation of TCR from tumor infiltrating lymphocytes (TIL) of patients (for example see; Linnemann C, Heemskerk B, Kvistborg P, Kluin R J, Bolotin D A, Chen X, Bresser K, Nieuwland M, Schotte R, Michels S, Gomez-Eerland R, Jahn L, Hombrink P, Legrand N, Shu C J, Mamedov I Z, Velds A, Blank C U, Haanen J B, Turchaninova M A, Kerkhoven R M, Spits H, Hadrup S R, Heemskerk M H, Blankenstein T, Chudakov D M, Bendle G M, Schumacher T N. High-throughput identification of antigen-specific TCRs by TCR gene capture. Nat Med. 2013 November; 19(11):1534-41; Rosati S F, Parkhurst M R, Hong Y, Zheng Z, Feldman S A, Rao M, Abate-Daga D, Beard R E, Xu H, Black M A, Robbins P F, Schrump D A, Rosenberg S A, Morgan R A. A novel murine T-cell receptor targeting NY-ESO-1. J Immunother. 2014 April; 37(3):135-46. However, all of these methods have the potential drawback that they identify TCRs which are only functional in a given HLA context, which limits their usefulness to certain patient populations.

The TCR is a heterodimeric cell surface protein of the immunoglobulin superfamily which is associated with invariant proteins of the CD3 complex involved in mediating signal transduction. TCRs exist in αβ and γδ forms, which are structurally similar but T cells expressing them have quite distinct anatomical locations and functions. The extracellular portion of the receptor consists of two membrane-proximal constant regions, and two membrane-distal variable regions bearing three polymorphic loops analogous to the complementarity determining regions (CDRs) of antibodies. It is these loops which form the binding site of the TCR molecule and determine peptide specificity. Specifically, CDR1 and CDR2 interact mostly with the MHC-molecules, whereas CDR3 interacts specifically with the peptide ligand presented by the MHC (Shore D A, Issafras H, Landais E, Teyton L, Wilson I A. The crystal structure of CD8 in complex with YTS156.7.7 Fab and interaction with other CD8 antibodies define the binding mode of CD8 alphabeta to MHC class I. J Mol Biol. 2008 Dec. 31; 384(5):1190-202; Borg N A, Ely L K, Beddoe T, Macdonald W A, Reid H H, Clements C S, Purcell A W, Kjer-Nielsen L, Miles J J, Burrows S R, McCluskey J, Rossjohn J. The CDR3 regions of an immunodominant T cell receptor dictate the ‘energetic landscape’ of peptide-MHC recognition. Nat Immunol. 2005 February; 6(2):171-80). Mathematical estimates of potential TCR diversity are in the range of 10¹²-10¹⁵ different TCR, however, in facilitating self-tolerance, thymic positive and negative selection decease the size of the naïve TCRαβ repertoire in an individual to approximately 2×10⁷ TCRs for each human (Davis, M. M. & Chien, Y. H. in Fundamental Immunology 341-366, Lippincott-Raven, Philiadelphia 1999); Arstila, T. P. et al., A direct estimate of the human αβ T cell receptor diversity. Science 1999: 286958-961.) Since the CDR3 regions of the TCR alpha and beta chains confer specificity to the interaction with the MHC-presented peptide, characterization of CDR3 sequence variation provides a measure of T-cell diversity in an antigen-selected T-cell repertoire (Liaskou E, Henriksen E K, Holm K, Kaveh F, Hamm D, Fear J, Viken M K, Hov J R, Melum E, Robins H, Olweus J, Karlsen T H, Hirschfield G M. High-throughput T-cell receptor sequencing across chronic liver diseases reveals distinct disease-associated repertoires. Hepatology. 2015 Aug. 7. doi: 10.1002/hep.28116; Fang H, Yamaguchi R, Liu X, Daigo Y, Yew P Y, Tanikawa C, Matsuda K, Imoto S, Miyano S, Nakamura Y. Quantitative T cell repertoire analysis by deep cDNA sequencing of T cell receptor α and β chains using next-generation sequencing. Oncoimmunology. 2015 Jan. 7; 3(12):e968467). Interestingly, there is a bias in the TCR repertoire, as often identical and near-identical TCR repertoires can be observed across different individuals, so called “public TCRs” (Miles J J, Douek D C, Price D A. Bias in the αβ T-cell repertoire: implications for disease pathogenesis and vaccination. Immunol Cell Biol. 2011 March; 89(3):375-87). Several specific molecular features of these public TCR sequences have been postulated (Venturi V, Price D A, Douek D C, Davenport M P. The molecular basis for public T-cell responses? Nat Rev Immunol. 2008 March; 8(3):231-8). The MHC class I and class II ligands are also immunoglobulin superfamily proteins but are specialized for antigen presentation, with a polymorphic peptide binding site which enables them to present a diverse array of short peptide fragments at the APC cell surface.

A number of papers describe the production of rTCR heterodimers which include the native disulphide bridge which connects the respective subunits (Garboczi, et al., (1996), Nature 384(6605): 134-41; Garboczi, et al., (1996), J Immunol 157(12): 5403-10; Chang et al., (1994), PNAS USA 91: 11408-11412; Davodeau et al., (1993), J. Biol. Chem. 268(21): 15455-15460; Golden et al., (1997), J. Imm. Meth. 206: 163-169; U.S. Pat. No. 6,080,840). However, although such TCRs can be recognized by TCR-specific antibodies, none were shown to recognize its native ligand at anything other than relatively high concentrations and/or were not stable.

In WO 99/60120, a soluble TCR is described which is correctly folded so that it is capable of recognizing its native ligand, is stable over a period of time, and can be produced in reasonable quantities. This TCR comprises a TCR alpha. or gamma chain extracellular domain dimerised to a TCR β or δ chain extracellular domain respectively, by means of a pair of C-terminal dimerisation peptides, such as leucine zippers. This strategy of producing rTCRs is generally applicable to all TCRs.

Immunization with NY-ESO-1 has been used to induce both antibody and CD8+ CTL responses, although little effect on cancer progression was observed in these studies (Jager et al., Proc Natl Acad Sci USA. 2000; 97:12198). Adoptive immunotherapy, the transfer of lymphocytes with high antitumor activity, can mediate the regression of large established tumors, but the generation of HLA-matched, reactive lymphocytes is difficult, expensive, and labor intensive (Rosenberg et al., N Engl J Med. 1988; 319:1676; Walter et al., N Engl J Med. 1995; 333:1038; Mackinnon et al., Blood. 1995; 86:1261; Papadopoulos et al., N Engl J Med. 1994; 330:1185; Dudley et al., J Immunother. 2003; 26:332; Dudley et al., Nat Rev Cancer. 2003; 3:666). Tumor-infiltrating lymphocytes have been used in cell transfer therapies and have been shown to recognize a variety of melanoma tumor-associated antigens (TAA). The most commonly recognized TAA in melanoma is the MART-1, a melanocyte differentiation antigen, which is expressed in ˜90% of melanomas, whereas NY-ESO-1 is expressed in ˜34% of melanomas (Chen et al., Proc Natl Acad Sci USA. 1997; 94:1914).

Zhao et al, (J. Immunol., 2005 Apr. 1; 174(7): 4415-4423) describes the isolation of TCRs specific to the NY-ESO-1 CT antigen and their use to construct retroviral vectors, which were shown to transfer anti-NY-ESO-1 effector functions to normal primary human T cells. rTCR gene vectors, directed against common TAA, have the potential to be used to treat large numbers of cancer patients with their own transduced T cells without the need to identify antitumor T cells uniquely from each patient. However, currently all approaches to develop ACT based on rTCR are HLA-dependent and thus limited to certain patient populations.

### SUMMARY

One aspect of the present disclosure provides a chimeric heterodimeric T cell receptor (TCR) polypeptide comprising a) a first polypeptide comprising a TCR β chain variable region, a TCR β chain constant region, and optionally a transmembrane domain and a cytoplasmic signaling domain; b) a second polypeptide comprising a TCR alpha chain variable region, a TCR alpha chain constant region, and optionally a transmembrane domain and a cytoplasmic signaling domain; wherein the heterodimeric TCR specifically binds to an NY-ESO-1/MHC complex, wherein the TCR beta chain variable region comprises the TCR beta chain variable region amino acid sequence set forth in SEQ ID NO:9, or a functional variant thereof having at least 85% identity thereto; wherein the TCR alpha chain variable region comprises the cognate TCR alpha chain variable region amino acid sequence set forth in SEQ ID NO:8, or a functional variant thereof having at least 85% identity thereto; and wherein there is at least one disulfide bond between the first polypeptide and the second polypeptide. In certain embodiments of the chimeric TCR, the beta chain variable region CDR3 comprises the amino acid CASRLAGQETQYF (SEQ ID NO: 4). In certain other embodiments of the chimeric heterodimeric TCR described herein, the first polypeptide and the second polypeptide do not comprise the transmembrane domain and the cytoplasmic signaling domain and thus the chimeric heterodimeric TCR is soluble. The present disclosure also provides nucleic acids comprising a polynucleotide sequence that encodes the chimeric heterodimeric TCRs described herein. In other embodiments the present disclosure provides expression vectors comprising the nucleic acids that encodes the chimeric heterodimeric TCRs described herein. In certain embodiments the expression vector is a retroviral vector, such as a lentiviral. In certain embodiments, the present disclosure also provides isolated cells comprising the nucleic acids described herein or the vectors described herein encoding any of the engineered TCRs, such as the chimeric heterodimeric TCRs described herein. In certain embodiments, the isolated cells are T cells.

The present disclosure also provides pharmaceutical compositions comprising the chimeric heterodimeric TCRs, or any of the other engineered TCRs described herein, or any of the vectors expressing the chimeric heterodimeric TCRs as described herein, or the nucleic acids encoding the chimeric heterodimeric TCRs described herein or the isolated cells modified to express the chimeric heterodimeric TCRs described herein.

Another aspect of the present disclosure provides a single chain TCR comprising a TCR beta chain variable region, a TCR alpha chain variable region, a constant region and optionally a transmembrane domain and a cytoplasmic signaling domain; wherein the TCR beta chain variable region CDR3 comprises an amino acid sequence selected from the group consisting of CASSLNRDYGYTF (SEQ ID NO: 2), CASSLNRDQPQHF (SEQ ID NO: 3) and CASRLAGQETQYF (SEQ ID NO: 4); wherein the single chain TCR is specific for an NY-ESO-1/MHC complex. In certain embodiments of the single chain TCR, the TCR beta chain variable region comprises a TCR beta chain variable region amino acid sequence set forth in SEQ ID NO:9, or a functional variant thereof having at least 85% identity thereto; and the TCR alpha chain variable region comprises the cognate TCR alpha chain variable region amino acid sequence as set forth in SEQ ID NO:8, or a functional variant thereof having at least 85% identity thereto.

In certain embodiments, the single chain TCR is a soluble single chain TCR. In this regard, the single chain TCR does not comprise the transmembrane domain and the cytoplasmic signaling domain.

Another aspect of the present invention provides a nucleic acid comprising a polynucleotide sequence that encodes a single chain TCR as described herein. In certain embodiments, the nucleic acid is comprised in an expression vector. In certain embodiments the expression vector is a retroviral vector, such as a lentiviral vector. The present disclosure also provides isolated cells comprising a nucleic acid or a vector encoding a single chain TCR as described herein. In certain embodiments, the isolated cell is a T cell. The present disclosure also provides pharmaceutical compositions comprising the single chain TCRs described herein, vectors encoding or otherwise expressing the single chain TCRs, nucleic acids encoding the single chain TCRs, and or the isolated cells expressing the single chain TCRs.

Another aspect of the present invention provides a method of treating or a method of inhibiting proliferation of an NY-ESO-1 cancer in a mammalian subject comprising administering to the subject a therapeutic composition, said composition comprising the isolated cells expressing an engineered TCR, such as a chimeric heterodimeric TCR or a single chain TCR as described herein; wherein the therapeutic composition is administered in an amount effective to treat the cancer in the subject.

Another aspect of the present disclosure provides a method of treatment comprising: (a) identifying a mammalian subject as likely to benefit from a NY-ESO-1 cancer therapy comprising determining in a sample from the mammalian subject the presence of (i) a polynucleotide encoding a TCR polypeptide comprising a TCR beta chain variable region complementarity determining region 3 (VβCDR3) that is specific for NY-ESO-1, wherein the vβ CDR3 comprises the amino acid sequence of CASSLNRDXXXXF (SEQ ID NO: 1) or CASRLAGQETQYF (SEQ ID NO: 4), or both; or (ii) a TCR polypeptide comprising a VβCDR3 that is specific for NY-ESO-1, wherein the VβCDR3 comprises the amino acid sequence of CASSLNRDXXXXF (SEQ ID NO: 1) or CASRLAGQETQYF (SEQ ID NO: 4) or both; wherein the presence of (i) and/or (ii) is indicative that the subject is likely to benefit from the NY-ESO-1 cancer therapy; and (b) administering the NY-ESO-1 cancer therapy to the mammalian subject. In certain embodiments of the methods, the VβCDR3 comprises an amino acid sequence selected from the group consisting of CASSLNRDYGYTF (SEQ ID NO: 2), CASSLNRDQPQHF (SEQ ID NO: 3) or CASRLAGQETQYF (SEQ ID NO: 4); or a combination of two or more of the foregoing VβCDR3. In certain embodiments the NY-ESO-1 cancer therapy comprises administering a vector encoding an NY-ESO-1 polypeptide. In certain embodiments, the vector comprises a dendritic cell targeting retroviral vector, such as a lentiviral vector. In further embodiments the method further comprises administering an adjuvant to the subject. In this regard, the adjuvant may be a glucopyranosyl lipid A (GLA). In certain embodiments the GLA is formulated in a stable oil in water emulsion or may be in an aqueous formulation. In certain embodiments, the NY-ESO-1 cancer therapy comprises administering to the subject a composition comprising GLA, said composition comprising:

(a) GLA of the formula:

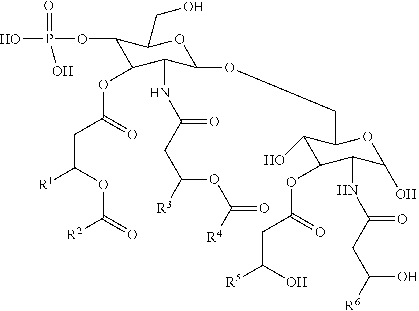

wherein:

R¹, R³, R⁵ and R⁶ are Cᵤ-C₂₀ alkyl; and

R² and R⁴ are C₁₂-C₂₀ alkyl; and

(b) a pharmaceutically acceptable carrier or excipient;

wherein the composition does not comprise antigen. In certain embodiments, R¹, R³, R⁵ and R⁶ are undecyl and R² and R⁴ are tridecyl. The methods herein may be used for the treatment of any mammal, in particular a human subject. In certain embodiments, the GLA composition is an aqueous formulation. In other embodiments, the composition is in the form of an oil-in-water emulsion, a water-in-oil emulsion, liposome, micellar formulation, or a microparticle. In one embodiment, the cancer to be treated by the method comprises a solid tumor. In this regard, the cancer may be selected from the group consisting of a sarcoma, prostate cancer, uterine cancer, thyroid cancer, testicular cancer, renal cancer, pancreatic cancer, ovarian cancer, oesophageal cancer, non-small-cell lung cancer, non-Hodgkin's lymphoma, multiple myeloma, melanoma, hepatocellular carcinoma, head and neck cancer, gastric cancer, endometrial cancer, colorectal cancer, cholangiocarcinoma, breast cancer, bladder cancer, myeloid leukemia and acute lymphoblastic leukemia. In certain embodiments, the composition is administered by subcutaneous, intradermal, intramuscular, intratumoral, or intravenous injection. In certain embodiments, the composition is administered in conjunction with one or more additional therapeutic agents or treatments. In one embodiment the therapeutic agent is an immune checkpoint inhibitor. In other embodiments the therapeutic agent is an antibody that activates a costimulatory pathway, such as an anti-CD40 antibody. In another embodiment, the therapeutic agent is a cancer therapeutic agent, such as a cancer therapeutic agent is selected from the group consisting of taxotere, carboplatin, trastuzumab, epirubicin, cyclophosphamide, cisplatin, docetaxel, doxorubicin, etoposide, 5-FU, gemcitabine, methotrexate, and paclitaxel, mitoxantrone, epothilone B, epidermal-growth factor receptor (EGFR)-targeting monoclonal antibody 7A7.27, vorinostat, romidepsin, docosahexaenoic acid, bortezomib, shikonin and an oncolytic virus. In another embodiment, the one or more additional therapeutic treatments is radiation therapy.

Another aspect of the present disclosure provides a method of identifying a mammalian subject that is likely to benefit from an NY-ESO-1 cancer therapy comprising: (a) determining in a sample from the mammalian subject the presence of (i) a polynucleotide encoding a TCR polypeptide comprising a VβCDR3 that is specific for NY-ESO-1, wherein the VβCDR3 comprises the amino acid sequence of CASSLNRDXXXXF (SEQ ID NO: 1) or CASRLAGQETQYF (SEQ ID NO: 4); or (ii) a TCR polypeptide comprising a VβCDR3 that is specific for NY-ESO-1, wherein the VβCDR3 comprises the amino acid sequence of CASSLNRDXXXXF (SEQ ID NO: 1) or CASRLAGQETQYF (SEQ ID NO: 4); wherein the presence of (i) and/or (ii) is indicative that the subject is likely to benefit from the NY-ESO-1 cancer therapy. In certain embodiments of the method, the VβCDR3 comprises an amino acid sequence selected from the group consisting of CASSLNRDYGYTF (SEQ ID NO: 2), CASSLNRDQPQHF (SEQ ID NO: 3) and CASRLAGQETQYF (SEQ ID NO: 4); or a combination of two or more of the foregoing VβCDR3.

An additional aspect of the present invention provides a method for detecting cells or tissue comprising an NY-ESO-1 peptide antigen presented on the cells or tissue in the context of an MHC complex, the method comprising: a) contacting the cells or tissue with at least one soluble TCR molecule or functional fragment thereof as described herein under conditions that form a specific binding complex between the presented NY-ESO-1 peptide antigen and the soluble TCR or fragment, b) washing the cells or tissue under conditions appropriate to remove any soluble TCR molecule or fragment not bound to the presented peptide antigen; and c) detecting the specific binding complex as being indicative of cells or tissue comprising the presented peptide antigen.

### BRIEF DESCRIPTION OF THE FIGURES

FIG. 1: Treatment with LV305 results in increased affinity of a polyclonal NY-ESO-1 specific T cell response. Cryopreserved PBMC from pre-Tx and post-Tx leukapheresis samples were thawed and used to set up the ELISPOT assay. 200,000 cells were plated into each well of the ELISPOT plate. Cells were treated with different concentrations of NY-ESO-1 peptide mix, which contains 43 of 15mer peptides that are overlapping by 11 amino acid. The concentration of peptides tested were 2.5 ug/mL (1670 nM), 0.5 ug/mL (334 nM), 0.1 ug/mL (60 nM), and 0.02 ug/mL (12 nM). Cells were incubated with the peptides or control treatment medium for 40 hr. The spots were counted by using an automated spot counter from C.T.L Technologies. The data symbol represents the average number of spot-forming units (SFU) per million PBMC in pre-Tx sample (empty circle) or post-Tx sample (filled square) at each of the tested concentration. The error bar represents standard deviation at each treatment condition.

FIG. 2: Tumor antigen-specific TCR sequences are enriched in post-Tx PBMC as compared to pre-Tx PBMC. In this study, the PBMC were collected from the patient before LV305 treatment and after three vaccinations with LV305. A pre-therapy tumor sample was also collected from the patient. The PBMC and tumor samples were subjected to DNA extraction and subsequent sequencing analysis of the T cell receptor (TCR) beta chain. The sequence similarity between pre-Tx and post-Tx PBMC was analyzed using scatter plot and the TCR sequences obtained from the tumor sample were also compared to the patients pre-Tx and post-Tx PBMC for similarity. The result showed that the TCR sequence from tumor samples, which contain tumor infiltrating lymphocytes that recognize tumor antigens, are enriched in the patient's post-Tx PBMC as compared to pre-Tx PBMC.

FIG. 3: Establishment of an oligoclonal culture from post-Tx PBMC through in vitro culture (IVS), that is highly enriched for NY-ESO-1 specific T. PBMC were collected from a patient Pt151006 after three vaccinations with LV305. The PBMC were cultured in OpTmizer T cell expansion medium (Invitrogen, Carlsbad, Calif.) with NY-ESO-1 overlapping peptides (0.5 ug/mL, JPT Technologies, Berlin, Germany) in the presence of IL-2 and IL-7 (10 ng/mL). After repeated stimulation, the PBMC culture was highly enriched for NY-ESO-1 specific T cells as measured by ELSIPOT assay. (A) Representative ELISPOT showing the secretion of IFN-γ of the oligoclonal T cells upon stimulation with NY-ESO-1 peptide pool. The cells (10K/well) were plated in triplicate in ELISPOT plates pre-coated with anti-IFNγ capture antibody (MabTech). The cells were treated with NY-ESO-1 peptide pool (2.5 ug/mL) or control medium (No Ag) and incubated in a CO₂ incubator for 40 hr. The cells were then washed off and the plate was incubated with a HRP-conjugated secondary antibody for 2 hours before TMB substrate was added. The number of spot forming units (SFU) per well was counted by an automated plate reader. Shown is the image of three wells with No Ag treatment (top row) and three wells with NY-ESO-1 treatment (bottom row). (B) Summary graph showing the number of SFU per well in the No Ag control and NY-ESO-1 peptide mix-treated wells. The column represents the Mean±SEM of each group. (C) Show are the top clones from the oligoclonal culture as determined by TCRβ deep sequencing analysis. Each column represents one clone with a unique TCRβ CDR3 sequence. The y-axis shows the relative frequency of each clone (percentage) among all sequence reads from the oligoclonal culture. The top 6 clones account for more than 90% of all the TCRs, indicating that the culture is highly oligoclonal.

FIG. 4: Several high-frequency TCRβ CDR3 sequences in the oligoclonal culture are enriched in post-Tx PBMC as compared to pre-Tx PBMC. Shown is a log-scaled scatter plot comparing the TCR sequences in pre-Tx PBMC (x-axis) to post-Tx PBMC (y-axis) of the same patient, from whom the NY-ESO-1 specific oligoclonal culture was derived. The sequences from the oligoclonal culture was overlaid onto the PBMC sequence. There is a skewing of the overlapping sequencing toward the y-axis, indicating that the NY-ESO-1 specific sequences are more frequently found in post-Tx PBMC than pre-Tx PBMC, as an indication that LV305 induced NY-ESO-1 specific T-cell responses.

FIG. 5: A TCRβ CDR3 clone that has a frequency of 20.5% in PT151006 IVS3 can be detected in PT151016 post-Tx PBMC. Shown is a log-scaled scatter plot comparing the TCR sequences of the oligoclonal culture from PT151006 IVS3 (y-axis) to the TCR sequences detected in post-Tx PBMC from a second patient, PT151016. The two clones that have high frequency in IVS3 are also detectable in post-Tx sample from PT151016. The box within the scatter plot shows the amino acid sequence of the CDR3 region of the TCRβ and the percent of frequency in PT151016 post-Tx PBMC (0.000298) and the percent of frequency in IVS3 (20.5). A similar analysis showed that this sequence is non-detectable in pre-Tx PBMC from PT151016.

FIG. 6: A TCRβ CDR3 clone that has a frequency of 8.5% in PT151006 IVS3 can be detected in PT151016 post-Tx PBMC. Shown is a log-scaled scatter plot comparing the TCR sequences of the oligoclonal culture from PT151006 IVS3 (y-axis) to the sequence in post-Tx PBMC from a second patient, PT151016. Two clones with a high frequency in IVS3 are also detectable in post-Tx sample from PT151016. The box within the scatter plot shows the amino acid sequence of the CDR3 region of the TCRβ and the percent of frequency in PT151016 post-Tx PBMC (0.000642) and the percent of frequency in IVS3 (8.52). Similar analysis showed that this sequence is non-detectable in pre-Tx PBMC from PT151016.

FIG. 7: A TCRβ CDR3 clone that has a frequency of 26.2% in PT151006 IVS3 can be detected in PT151014 post-Tx PBMC. Shown is a log-scaled scatter plot comparing the TCR sequences of the oligoclonal culture from PT151006 IVS3 (y-axis) to the sequence in post-Tx PBMC from PT151014. The clone that have the highest frequency in IVS3 (26.2%) is also detectable in post-Tx sample from PT151014. The box within the scatter plot shows the amino acid sequence of the CDR3 region of the TCRβ and the percent of frequency in PT151014 post-Tx PBMC (0.00076) and the percent of frequency in IVS3 (26.2). Similar analysis showed that this sequence is non-detectable in pre-Tx PBMC from PT151014.

FIG. 8: frequency of three public TCRβ CDR3 sequences in pre-Tx and post-Tx PBMC from eight sarcoma patients. The bar graph shows the frequency of public TCR in pre-Tx (hatched bar) and post-Tx (black bar) PBMC from each of the eight tested patients. All patients have received LV305 treatment. The amino acid sequence of the relevant TCRβ CDR3 is included in the top of each graph. See also Tables 1-3.

FIG. 9: Public TCRs that are shared between patients have different nucleotide sequences in each individual. Shown are the nucleotide sequences and amino acid sequences for the three public TCR from three patients. There is no complete homology at nucleotide level between different patients even though the sequences are the same at amino acid level. This is consistent with the concept of convergent recombination, which has been proposed for the generation of public TCR (Venturi et al., Nat Rev Immunol., 2008).

FIG. 10: Public TCRβ CDR3 sequences have a shorter length and fewer nontemplated nucleotide additions at the VJD junction region. (A) Shown is the TCRβ CDR3 length distribution in IVS3 (black column), the oligoclonal NY-ESO-1 specific T-cell in vitro culture from Pt151006, and the TCRβ CDR3 length distribution in the post-Tx PBMC from the same patient (hatched column). (B) The nucleotide sequence (CDR3 encoding sequence is underlined) and amino acid sequence of the CDR3 of the three public TCRs. The number of deletions and additions in the junction regions are also listed for each TCR. All three public TCR have the same CDR3 nucleotide length (n=39). These TCRs also have relatively few non-templated nucleotide additions at the junction region. One of the public TCRs we identified, CASRLAGQETQYF (SEQ ID NO: 4), had no nucleotide addition at either the N1 (VD) and N2 (DJ) insertion sites. The shorter CDR3 length and limited nt addition support the conclusion that these TCRs are public TCRs.

FIG. 11: The sequence of the full TCRα (SEQ ID NO: 8) and TCRβ (SEQ ID NO: 9) variable regions for one of the identified public TCR CDR3 CASRLAGQETQYF (SEQ ID NO: 4). This CDR3 sequence has a 26.2% frequency in IVS3 and is also shared in PT151014 and PT151050, as listed in Table 3. The annotation shown is standard annotation available from the IMGT database (The international ImMunoGeneTics information system; internet address at IMGT (dot) org). A BLAST search of the TCRα sequence indicates homology with a known NY-ESO-1 specific TCRα sequence.

FIG. 12: An alignment of the polynucleotide sequences encoding public TCRβ CDR3s identified from different cancer patients shows different nucleic acid sequences from different patients encoding the same CDR3 amino acid sequence. The nucleotide sequence SEQ ID NOs are as follows: 1st public TCRβ CDR3-PT006: SEQ ID NO: 5; PT016: SEQ ID NO: 10; PT050: SEQ ID NO: 11; 2^{nd} public TCRβ CDR3-PT 006: SEQ ID NO: 6; PT 016: SEQ ID NO: 12; PT 050; SEQ ID NO: 13; 3^{rd} public TCRβ CDR3-PT 006: SEQ ID NO: 7; PT 016: SEQ ID NO: 14; PT 050: SEQ ID NO: 15. (see also FIG. 9)

FIG. 13 shows the amino acid sequence of a TCRβ chain (SEQ ID NO: 16) identified from an NY-ESO-1 expanded T cell culture (IVS3) expanded from PBMCs from a cancer patient. The sequence was isolated as described in Example 11.

FIG. 14: MCC patient G2 expresses NY-ESO-1 and the expression level decreases after treatment with G100. Tumor biopsy was taken at baseline prior to intratumoral G100 treatment and at 4 weeks after treatment with G100. RNA was extracted from snap frozen biopsy tissue and 200 ng of RNA was used for nanostring gene expression analysis by using the human panCancer Immune Profiling kit. Shown is the expression of CTAG1B gene, which encodes NY-ESO-1, the cancer testis antigen. The Y-axis shows the binding density, which reflects the expression level of the gene. The expression level was lower in post-G100 sample as compared to baseline.

FIG. 15: T cells with public TCRs are detectable after treatment with G100 in a lymphnode biopsy of MCC patient G2. A tumor biopsy was taken at baseline prior to intratumoral G100 treatment and at 4 weeks after treatment with G100. DNA was extracted from snap frozen biopsy tissue and used for deep sequencing analysis of the CDR3 region of the TCR beta chain. Shown are two scatter plots showing the sequence overlaps between tumor biopsies from patient G2 and TCR isolated from an oligoclonal, NY-ESO-1 stimulated T-cell culture obtained from patient 151006 in the LV305 trial. Graph (A) shows the correlation between pre-G100 sample (X-axis) and the NY-ESO-1 specific TCRs from LV305 patient 151006 (Y-axis). Graph (B) shows the correlation between post-G100 sample (X-axis) and the NY-ESO-1 specific TCRs from LV305 patient 151006 (Y-axis). Two public CDR3 sequences that were non-detectable in a pre-G100 lymph node biopsy with MCC were detectable in a biopsy of a draining lymph node post-G100 treatment.

FIG. 16. The three public TCRβ CDR3 sequences were detected in patients from the anti-CTLA4 clinical trial. Shown are the frequency of the 3 public CDR3 amino acid sequences in the 21 patients on the anti-CTLA4 trial. The numbers on the X axis represent patient number. There are two columns associated with each number. The column on the left indicates frequency in pre-Tx PBMC. The column on the right indicates frequency in post-Tx PBMC.

FIG. 17. Patient 151006 and patient 151119 use different TCRβ V-genes for the same CDR3 sequence, CASSLNRDQPQHF (SEQ ID NO:3). The majority of TCRβ use the V07-07 gene. However, a small percentage of the TCRβ receptors use V07-08, V07-06, V07-09, V07-02, V07-03, V07-04, or V11-02. In patient 151119, only V07-08 is used for this TCR CDR3. Both patients use the same TCRβ J-gene, J01-05.

FIG. 18: This figure shows that one of the public TCRβ CDR3 CASSLNRDQPQHF (SEQ ID NO:3) was encoded by at least three different nucleotide sequences in patient PT151006. The polynucleotide sequences are provided in SEQ ID NOs:

FIG. 19. Patients from different clinical trials have different nucleotide sequences and different TCRβ V-gene usage for the same CDR3 amino acid sequences. For the first public TCR, CASSLNRDYGYTF (SEQ ID NO:2), PT151006 (from LV305 trial) and C131-001 (from C131 trial) and patient G2-C1W4B (from the G100-MCC trial)-use TCRβ V07-08, TCRβ V02-01, and TCRβ V28-01, respectively. For the second public TCR, CASSLNRDQPQHF (SEQ ID NO:3), PT151006 use TCRβ V07-07. Patient 131-013 uses three different TCRβ V, V05-08, V13-01, and V05-05. For the third public TCR, CASRLAGQETQYF (SEQ ID NO:4), PT151006, C131-001, and G2-C1W4B use TCRβ V28-01, TCRβ V06-06, and TCRβ V25-01, respectively.

FIG. 20. The NY-ESO-1 specific T cell culture from PT151006 (IVS3) are CD4 T cells. Shown are side-by-side staining of IVS3 cell culture (bottom row) with PBMC from a normal donor (top row). The cells were stained with anti-CD3-pacific blue (PB), anti-CD4-FITC, anti-CD8-PerCP, and anti-CD56-APC. Samples were acquired on a BD LSRII flow cytometer. Data analysis was done using the FlowJo software. The lymphocytes population was first gated on the FSC/SSC plot, then CD4 T cells were gated as CD3+CD4+ lymphocytes and CD8 T cells were gated as CD3+CD8+ lymphocytes. The NK cells were gated as CD3-CD56+ lymphocytes. The control donor PBMC has the expected percentages of CD4, CD8 T cells and NK cells, as normally observed in healthy donor PBMC (top row). In contrast, the cultured cells from PT151006-IVS3 show a lack of NK cells and CD8 T cells and only contains CD4 T cells (bottom row).

### BRIEF DESCRIPTION OF THE SEQUENCE IDENTIFIERS

SEQ ID NO:1 is the amino acid sequence of a public TCR CDR3 consensus sequence CASSLNRDXXXXF.

SEQ ID NO:2 is the amino acid sequence of a first public TCR CDR3 CASSLNRDYGYTF.

SEQ ID NO: 3 is the amino acid sequence of a second public TCR CDR3 CASSLNRDQPQHF.

SEQ ID NO: 4 is the amino acid sequence of a third public TCR CDR3 CASRLAGQETQYF.

SEQ ID NO: 5 is a polynucleotide sequence encoding the TCR V region including the CDR3 amino acid sequence set forth in SEQ ID NO: 2.

SEQ ID NO: 6 is a polynucleotide sequence encoding the TCR V region including the CDR3 amino acid sequence set forth in SEQ ID NO: 3.

SEQ ID NO: 7 is a polynucleotide sequence encoding the TCR V region including the CDR3 amino acid sequence set forth in SEQ ID NO: 4.

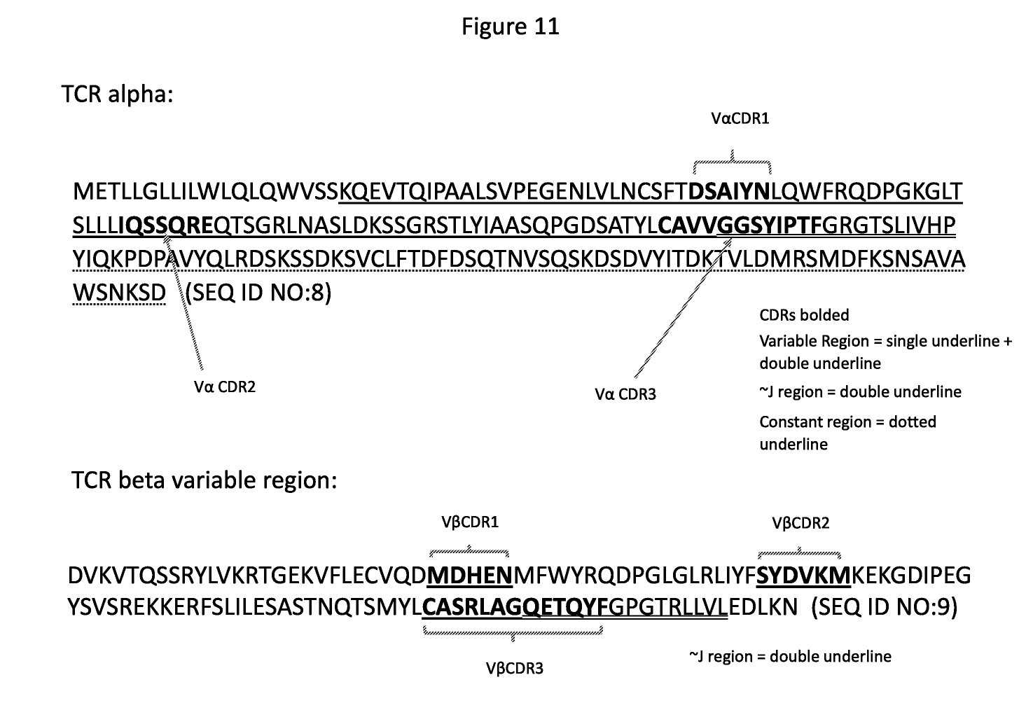

SEQ ID NO: 8 is the amino acid sequence of the alpha chain of a public TCR as shown in FIG. 11.

SEQ ID NO: 9 is the amino acid sequence of the beta chain variable region of a public TCR as shown in FIG. 11. This public TCR has the V β CDR3 sequence as shown in SEQ ID NO: 4.

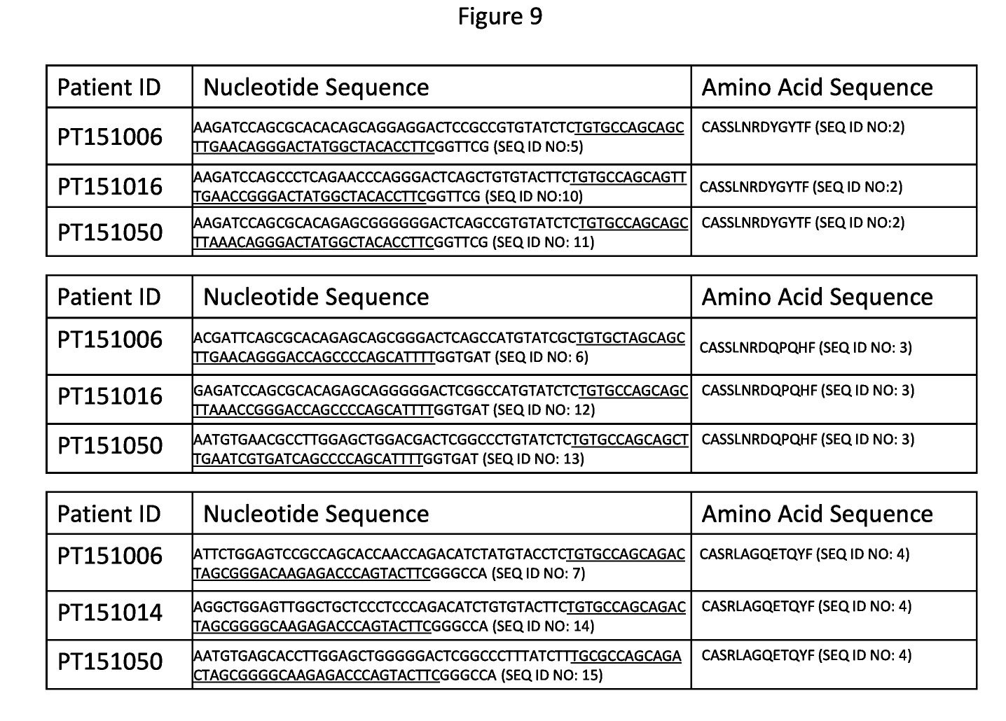

SEQ ID NO: 10 is a polynucleotide sequence encoding the TCR V region including the CDR3 amino acid sequence set forth in SEQ ID NO: 2 (see FIG. 9).

SEQ ID NO: 11 is a polynucleotide sequence encoding the TCR V region including the CDR3 amino acid sequence set forth in SEQ ID NO: 2 (see FIG. 9).

SEQ ID NO: 12 is a polynucleotide sequence encoding the TCR V region including the CDR3 amino acid sequence set forth in SEQ ID NO: 3 (see FIG. 9).

SEQ ID NO: 13 is a polynucleotide sequence encoding the TCR V region including the CDR3 amino acid sequence set forth in SEQ ID NO: 3 (see FIG. 9).

SEQ ID NO: 14 is a polynucleotide sequence encoding the TCR V region including the CDR3 amino acid sequence set forth in SEQ ID NO: 4 (see FIG. 9).

SEQ ID NO: 15 is a polynucleotide sequence encoding the TCR V region including the CDR3 amino acid sequence set forth in SEQ ID NO: 4 (see FIG. 9).

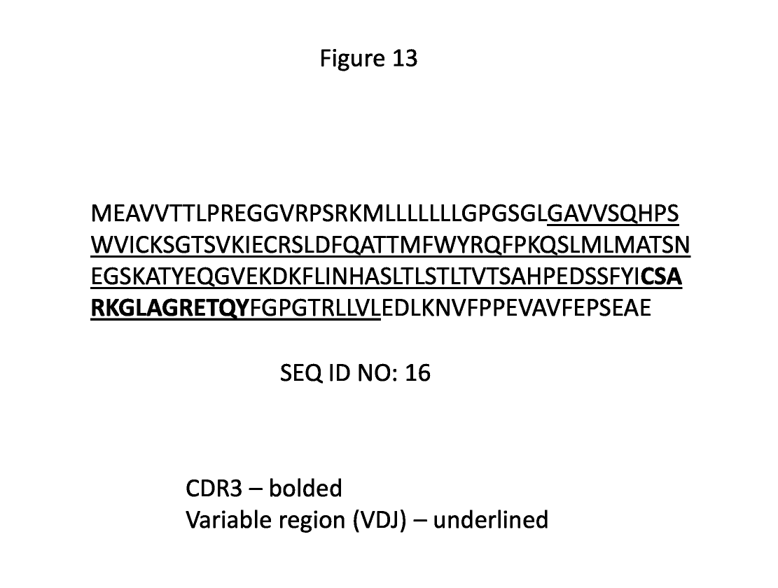

SEQ ID NO: 16 is the amino acid sequence of a TCRβ chain identified from an NY-ESO-1 expanded T cell culture (IVS3) expanded from PBMCs from a cancer patient as described in Example 11. The sequence is shown in FIG. 13 and annotated according to methods outlined at the IMGT database website.

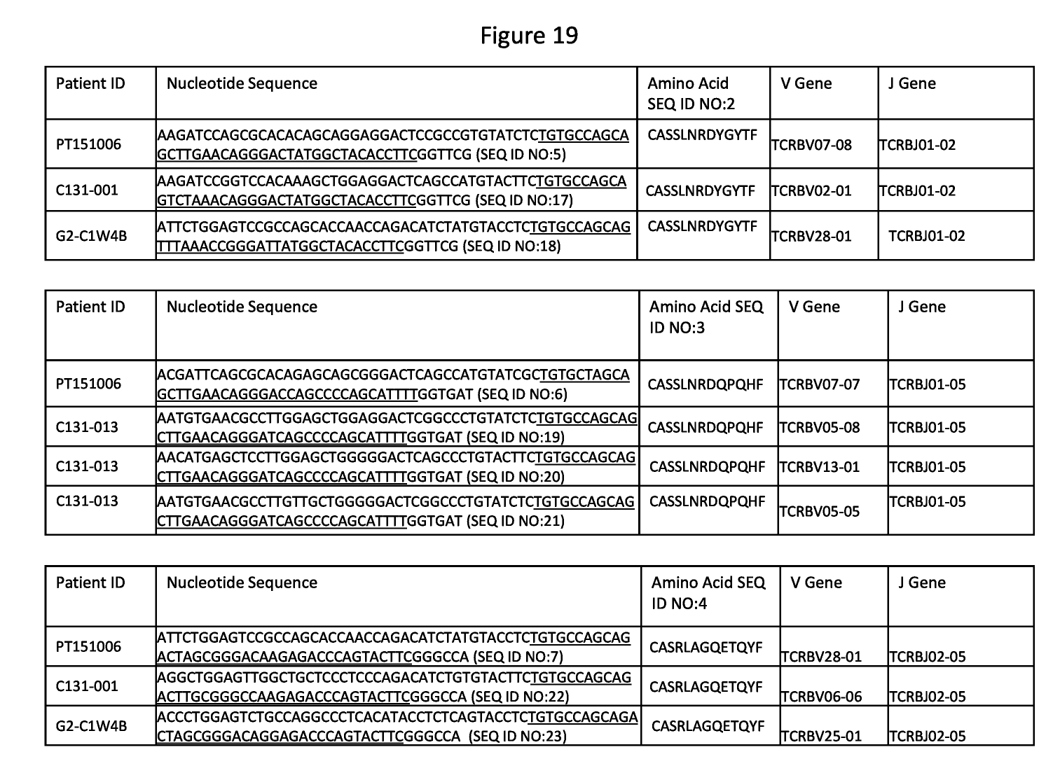

SEQ ID NO:17-23 are nucleotide sequences encoding public TCRβ CDR3 sequences from patients C131-001, G2-C1WB4, C131-013 as shown in FIG. 19.

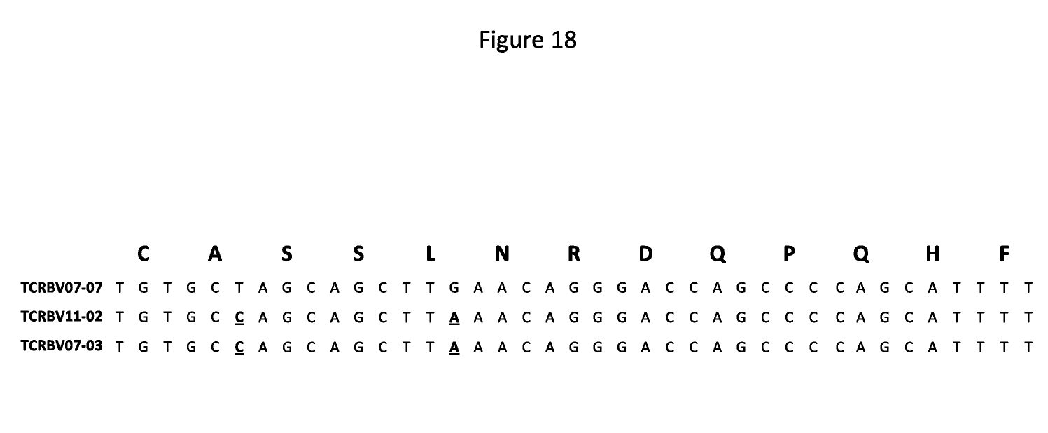

SEQ ID NO:24-26 are nucleotide sequences from PT151006 all encoding public TCRβ CDR3 sequence CASSLNRDQPQHF (SEQ ID NO:3) as shown in FIG. 18.

### DETAILED DESCRIPTION

The present disclosure is based in part on the discovery of a panel of public NY-ESO-1 specific TCR amino acid sequences that are shared between cancer patients of different MHC class I haplotypes. The term “public TCR” as used herein refers to a TCR sequence, in particular a TCRβ chain variable region CDR3 (VβCDR3) amino acid sequence, shared among multiple individuals (Venturi et al., Nat Rev Immunol. 2008; 8(3):231-238). As demonstrated in the Examples, the frequency of the public TCRs identified increased after treatment with NY-ESO-1 specific therapeutic immunization as well as after treatment with G100, a synthetic TLR4 agonist.

### Engineered TCR

The αβ TCR is a membrane-anchored heterodimeric protein comprising a highly variable alpha (α) and beta (β) chain expressed as part of a complex with the invariant CD3 chain molecules. Janeway C A Jr, Travers P, Walport M et al. (2001). Immunobiology: The Immune System in Health and Disease. 5th edition. Glossary: Garland Science. Each chain of the T cell receptor is comprised of two extracellular domains: Variable (V) region and a Constant (C) region. The Constant region is proximal to the cell membrane, followed by a transmembrane region and a short cytoplasmic tail, while the Variable region binds to the peptide/MHC complex. The variable domain of both the TCR α-chain and β-chain each have three hypervariable or complementarity determining regions (CDRs), whereas the variable region of the β-chain has an additional area of hypervariability (HV4) that does not normally contact antigen and, therefore, is not considered a CDR. The TCR is membrane bound, however, it is not able to mediate signal transduction itself due to its short cytoplasmic tail, so the TCR requires CD3 and zeta to carry out the signal transduction.

The present invention provides for engineered T cell receptors specific for NY-ESO-1/MHC complex and isolated cells expressing the engineered T cell receptors described herein. In certain embodiments described herein, the engineered TCR comprises the alpha chain variable region and the beta chain variable region as provided in SEQ ID NO:8 and 9 respectively. In other embodiments described herein, the engineered TCR comprises the beta chain variable region as provided in SEQ ID NO: 9 and an alpha chain pair such that the alpha/beta pair form a functional TCR that recognizes an NYESO1 epitope in the context of MHC. In particular embodiments, the VβCDR3 of the engineered TCRs described herein comprises an amino acid sequence comprising CASSLNRDXXXXF, wherein X is any amino acid (SEQ ID NO: 1). In some embodiments, the VβCDR3 comprises an amino acid sequence selected from the group consisting of CASSLNRDYGYTF (SEQ ID NO: 2) and CASSLNRDQPQHF (SEQ ID NO: 3), each of which are examples of sequences that fall within the consensus sequence of SEQ ID NO: 1. In another embodiment, the VβCDR3 comprises an amino acid sequence set forth in SEQ ID NO: 4.

In one aspect of the present invention, the engineered TCR is a heterodimeric TCR. A heterodimeric TCR comprises two polypeptides connected by at least one disulfide bond. One polypeptide in the heterodimeric TCR comprises an alpha chain variable region and an alpha chain constant region. In one embodiment, the alpha chain polypeptide optionally includes a transmembrane domain. In certain embodiments, the alpha chain polypeptide optionally includes a transmembrane domain and a cytoplasmic domain (e.g., intracellular signaling domain such as CD3 zeta chain signaling domain and optionally a costimulatory domain). The second polypeptide in the heterodimeric TCR comprises a beta chain variable region and a beta chain constant region, and optionally a transmembrane domain. In certain embodiments in the heterodimeric TCR, the second polypeptide comprises a beta chain variable region and a beta chain constant region and optionally a transmembrane domain and a cytoplasmic domain (e.g., an intracellular signaling domain and optionally a costimulatory domain). In those embodiments where the first and second polypeptides of the heterodimeric TCR do not contain the transmembrane domain and the cytoplasmic domain (e.g., intracellular signaling domain), the heterodimeric TCR is a soluble heterodimer. In one embodiment, the heterodimeric TCR may comprise one or more modifications to stabilize expression and minimize interaction with the native TCR that may be present in a host cell. In certain embodiments, the heterodimeric TCR is a chimeric heterodimeric TCR.

In one aspect of the present invention, the engineered T cell receptor is a chimeric T cell receptor (TCR). As used herein, the term “chimeric” refers to a molecule, e.g., a TCR, composed of parts of different origins. A chimeric molecule, as a whole, is non-naturally occurring, e.g., synthetic or recombinant, although the parts which comprise the chimeric molecule can be naturally occurring.

In some embodiments, the engineered TCR disclosed herein is a chimeric heterodimeric TCR. A chimeric heterodimeric TCR comprises two polypeptides connected by at least one disulfide bond. One polypeptide in the heterodimeric TCR comprises an alpha chain variable region and an alpha chain constant region. The alpha chain polypeptide optionally includes a transmembrane domain, or optionally a transmembrane domain and a cytoplasmic domain (e.g., an intracellular signaling domain such as a CD3 zeta chain signaling domain, with or without a costimulatory domain). The second polypeptide in the chimeric heterodimeric TCR comprises a beta chain variable region and a beta chain constant region, and optionally a transmembrane domain, or optionally a transmembrane domain and a cytoplasmic domain (e.g., an intracellular signaling domain with or without a costimulatory domain). In those embodiments where the first and second polypeptides of the chimeric heterodimer TCR do not contain the transmembrane domain and the cytoplasmic domain, the chimeric heterodimeric TCR is a soluble chimeric heterodimer.

Polypeptide chains of TCRs are known in the art.

The engineered TCRs described herein comprise an antigen binding domain generally composed of at least a portion of a beta chain variable region and at least a portion of an alpha chain variable region, wherein the antigen binding domain is specific for or specifically binds to NY-ESO-1/MHC. The term “specifically binds,” as used herein refers to the ability of the TCR to recognize a specific antigen in the context of an MHC, but that does not substantially recognize or bind irrelevant antigen/MHC in a sample. In some instances, the terms “specific binding” or “specifically binding,” can be used in reference to the interaction of a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

Single chain TCRs (see, e.g., US20100113300) are also contemplated. Briefly stated, a single chain (“sc-”) TCR molecule includes an alpha chain variable region (Vα) and a beta chain variable region (Vβ) covalently linked through a suitable peptide linker sequence. For example, the Vα can be covalently linked to the VP through a suitable peptide linker sequence fused to the C-terminus of the Vα and the N-terminus of the Vβ. The scTCR of the present invention may have a structure Vα-L-Vβ or may be in the other orientation, e.g. Vβ-L-Vα. In certain embodiments, the scTCR further comprises a constant domain (also referred to as constant region). In a further embodiment, the scTCR further comprises a constant domain, a transmembrane domain and a cytoplasmic domain. In one embodiment, the cytoplasmic domain comprises an intracellular signaling domain with or without a costimulatory domain. The Vα and Vβ of the sc-TCR fusion protein are generally about 200 to 400 amino acids in length, or about 300 to 350 amino acids in length, and will be at least 90% identical, and preferably 100% identical to the Vα and Vβ of a naturally-occurring TCR, such as the Vα and Vβ amino acid sequences provided herein that are specific for NY-ESO-1/MHC complex. By the term “identical” is meant that the amino acids of the Vα or Vβ are 100% identical to the corresponding naturally-occurring TCR Vβ or Vα.

In some embodiments, the engineered TCR described herein comprises an intracellular signaling domain (e.g., CD3 zeta chain signaling domain). The intracellular signaling domain, which may also be referred to as the cytoplasmic signaling domain, of an engineered TCR described herein is responsible for activation of at least one of the normal effector functions of the immune cell that expresses the engineered TCR. The term “effector function” refers to a specialized function of a cell. Effector function of a T cell, for example, may be cytolytic activity or helper activity including the secretion of cytokines. Thus the term “intracellular signaling domain” or “cytoplasmic signaling domain” refers to the portion of a protein that transduces the effector function signal and directs the cell to perform a specialized function. While usually the entire intracellular signaling domain can be employed, in many cases it is not necessary to use the entire signaling domain. To the extent that a truncated portion of the intracellular signaling domain is used, such truncated portion may be used in place of the intact full length signaling domain as long as it transduces the effector function signal. The term intracellular signaling domain is thus meant to include any truncated portion of the intracellular signaling domain sufficient to transduce the effector function signal.

Examples of intracellular signaling domains for use in the engineered TCRs described herein include the cytoplasmic sequences of the T cell receptor (TCR) and co-receptors that act in concert to initiate signal transduction following antigen receptor engagement, as well as any derivative or variant of these sequences and any synthetic sequence that has the same functional capability. Also contemplated herein are NK signaling molecules. Thus, contemplated for use herein as intracellular signaling domains are the polypeptides constituting the CD3 complex which are involved in the signal transduction, e.g., the γ, δ, ε, ζ, and η, CD3 chains. Among the polypeptides of the TCR/CD3 (the principal signaling receptor complex of T cells), especially promising are the zeta and its eta isoform chain, which appear as either homo- or hetero-S—S-linked dimers, and are responsible for mediating at least a fraction of the cellular activation programs triggered by the TCR recognition of ligand (Weissman, A. et al. EMBO J. 8:3651-3656 (1989); Bauer, A. et al. Proc. Natl. Acad. Sci. USA 88:3842-3846 (1991)). Further examples of intracellular signaling domains for use herein include the MB1 chain (CD79A), B29, Fc RIII and Fc RI and the like. Intracellular signaling portions of other members of the families of activating proteins can also be used, such as FcγRIII and FccRI. See Gross et al., FASEB J 6:3370, 1992; Stancovski, I. et al., J. Immunol. 151:6577, 1993; Moritz, D. et al., Proc. Natl. Acad. Sci. U.S.A. 91:4318, 1994; Hwu et al., Cancer Res. 55:3369, 1995; Weijtens, M. E. et al., J. Immunol. 157:836, 1996; and Hekele, A. et al., Int. J. Cancer 68:232, 1996; for disclosures of various alternative transmembrane and intracellular domains contemplated for use herein. Additional examples include the intracellular signaling domains of any one of the IL-2 receptor (IL-2R) p55 (.alpha.) or p75 (.beta.) or .gamma. chains, especially the p75 and .gamma. subunits which are responsible for signaling T cell and NK proliferation.

It is known that signals generated through the TCR alone are insufficient for full activation of the T cell and that a secondary or costimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or costimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use as intracellular signaling domains herein include those derived from TCR, FcR γ, FcR β, CD3 γ, CD3 delta, CD3 epsilon, CD5, CD22, CD79a, CD79b, and CD66d. In certain particular embodiments, the cytoplasmic signaling domain in the engineered TCR used herein comprises a cytoplasmic signaling sequence derived from CD3 zeta. The zeta chain portion sequence useful herein includes the intracellular domain. This domain, which spans amino acid residues 52-163 of the human CD3 zeta chain, can be amplified using standard molecular biology techniques.

In some embodiments, the cytoplasmic domain of the engineered TCR can be designed to comprise the CD3-zeta signaling domain by itself or combined with any other desired cytoplasmic domain(s) useful in the context of the TCRs for use herein. The “costimulatory signaling region” or “costimulatory domain” refers to a portion of the engineered TCR comprising the intracellular domain, or a functional fragment thereof, of a costimulatory molecule. Thus, the cytoplasmic domain of the engineered TCRs described herein may comprise an intracellular signaling domain and a costimulatory domain.

“Costimulatory ligand,” includes a molecule on an antigen presenting cell (e.g., an aAPC, dendritic cell, B cell, and the like) that specifically binds a cognate costimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A costimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A costimulatory ligand also encompasses, inter alia, an antibody that specifically binds with a costimulatory molecule present on a T cell, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40L, PD-1, DAP-10, ICOS, lymphocyte function-associated antigen-1 (LFA-1), CD2, CD7, LIGHT, NKG2C, B7-H3, and a ligand that specifically binds with CD83, and the like. Particularly illustrative costimulatory domains contemplated for use with the TCRs described herein are derived from 4-1BB and CD28 however, other costimulatory domains derived from other costimulatory molecules are within the scope of the invention.

The intracellular signaling sequences and costimulatory sequences within the cytoplasmic domain of the engineered TCRs disclosed herein may be linked to each other in any order in which each portion functions to signal properly. In certain embodiments, the costimulatory region, when present, is just on the cytoplasmic side of the TM domain, followed by a signaling domain (e.g. the signaling domain of CD3 zeta). In other embodiments, the order is reversed, with the signaling portion just next to the TM domain, followed by a costimulatory domain, when present. Optionally, a short oligo- or polypeptide linker, in certain embodiments, about 1, 2, 3, 4, 5, 6, 7, 8, 9, 10, 11, 12, 13, 14, or 15 amino acids in length may form the linkage. A glycine-serine doublet provides a particularly suitable linker. Any of a variety of linkers can be used and are known to the skilled person.

As noted herein, the engineered TCRs also comprise, in some embodiments, a transmembrane (TM) domain to anchor them to the surface of the host cell (e.g., T cell, NK cell). The TM can be derived from a TCR alpha chain, a TCR beta chain, from the CD3 zeta chain or can be derived from another transmembrane molecule, such as CD28 or CD4. As would be recognized by the skilled person, any TM domain that functions properly to anchor the chimeric receptor to the membrane can be used. With respect to the transmembrane domain, the chimeric TCR can be designed to comprise a transmembrane domain that is fused to the extracellular domain of the chimeric TCR. In one embodiment, the transmembrane domain that naturally is associated with one of the domains in the chimeric TCR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it may comprise predominantly hydrophobic residues such as leucine and valine. In certain embodiments, a triplet of phenylalanine, tryptophan and valine will be found at each end of a synthetic transmembrane domain. Optionally, a short linker, in certain embodiments between 1 or 2 and about 10, 11, 12, 13, 14, or 15 amino acids in length may form the linkage between the transmembrane domain and the cytoplasmic signaling domain of the chimeric TCR. A glycine-serine doublet provides a particularly suitable linker.

In certain embodiments, the transmembrane domain in the chimeric TCR is the CD8 transmembrane domain. In some instances, the transmembrane domain of the chimeric TCR for use herein comprises the CD8 hinge domain. In certain embodiments, the transmembrane domain is the CD28 transmembrane domain, in particular in the embodiment where the costimulatory region is derived from CD28 since it is largely as a matter of convenience to minimize the number of amplification/cloning steps that need to be performed. Thus, in certain embodiments, the TM domain may be derived from the same molecule as a costimulatory or intracellular signaling domain of the chimeric TCR. However, this is not necessary and the TM domain may be derived from any suitable transmembrane protein, including, but not limited to, the CD8 and CD3 zeta transmembrane domains.

TCR constant domains: the constant domains for use in the engineered TCRs of the present invention may be derived from the TCR alpha chain or the TCR beta chain. Such constant domains are known in the art and available from public sequence databases.

In certain embodiments one or more disulfide bonds may link amino acid residues of the constant domain sequences included in the engineered TCRs of the present invention. In one embodiment, the disulfide bond is between cysteine residues corresponding to amino acid residues whose beta carbon atoms are less than 0.6 nm apart in native TCRs. For example, the disulfide bond may be between cysteine residues substituted for Thr 48 of exon 1 of TRAC*01 and Ser 57 of exon 1 of TRBC1*01 or TRBC2*01 or the non-human equivalent thereof. Other sites where cysteines can be introduced to form the disulfide bond are the following residues in exon 1 of TRAC*01 for the TCR .alpha. chain and TRBC1*01 or TRBC2*01 for the TCR .beta. chain:

| TCR alpha chain | TCR beta chain | native beta carbon separation (nm) |
|---|---|---|
| Thr 45 | Ser 77 | 0.533 |
| Tyr 10 | Ser 17 | 0.359 |
| Thr 45 | Asp 59 | 0.560 |
| Ser 15 | Glu 15 | 0.59 |

In addition to the non-native disulfide bond referred to above, the dimeric TCR or scTCR form of the TCRs of the invention may include a disulfide bond between residues corresponding to those linked by a disulfide bond in native TCRs.

Soluble TCR

In some embodiments, the engineered TCR is a soluble TCR. Generally, “soluble TCRs” comprise TCR chains which have been truncated to remove the transmembrane regions thereof. For example, WO 03/020763 describes the production and testing of soluble TCRs having a non-native disulfide interchain bond to facilitate the association of the truncated TCR chains. Details of other potentially suitable soluble TCR designs can be found in WO 99/60120 which described the production of non-disulfide linked truncated TCR chains which utilize heterologous leucine zippers fused to the C-termini thereof to facilitate chain association, and WO 99/18129 which describe the production of single-chain soluble TCRs comprising a TCR Vα chain covalently linked to a TCR Vβ chain via a peptide linker. Boulter et al. also describe methods for producing soluble functional and stable TCR heterodimers (see Boulter et al., Protein Eng 2003, 16:707-711). In further embodiments, the soluble TCRs described herein can be chimeric, e.g., fused to a heterologous protein, such as IL-2 or other cytokines, Fc domain of an antibody, and the like. Illustrative soluble TCR fusion proteins are described for example in Cancer Immunol Immunother. 2004 April; 53(4):345-57; J Immunol. 2005 Apr. 1; 174(7):4381-8; Clin Immunol. 2006 October; 121(1):29-39.

### Functional Variants and Portions of Engineered TCR

Functional variants of the TCRs described herein are also contemplated. The term “functional variant” as used herein refers to a TCR having substantial or significant sequence identity or similarity to a parent TCR, which functional variant retains the biological activity of the TCR of which it is a variant. Functional variants encompass, for example, those variants of the TCR described herein that retain the ability to specifically bind to an NY-ESO-1 polypeptide/MHC complex for which the parent TCR has antigenic specificity or to which the parent polypeptide or protein specifically binds, to a similar extent, the same extent, or to a higher extent, as the parent TCR. In some embodiments, the functional variant comprises a beta chain variable domain comprising an amino acid sequence that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the beta chain variable domain amino acid sequence of the parent TCR, such as the beta chain variable domain amino acid sequences set forth in the sequence listing provided herein. In some embodiments, the functional variant comprises a VβCDR3 amino acid sequence that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the VβCDR3 amino acid sequence set forth in SEQ ID NO: 1, SEQ ID NO: 2 or SEQ ID NO: 3.

In some embodiments, the functional variant comprises an alpha chain variable domain comprising an amino acid sequence that is at least 50%, 60%, 70%, 80%, 81%, 82%, 83%, 84%, 85%, 86%, 87%, 88%, 89%, 90%, 91%, 92%, 93%, 94%, 95%, 96%, 97%, 98%, or 99% identical to the alpha chain variable domain amino acid sequence of the parent TCR, such as the alpha chain variable domain amino acid sequences set forth in the sequence listing provided herein.

In some embodiments, the amino acid sequence of the functional variant can comprise, for example, the amino acid sequence of the parent TCR with at least one conservative amino acid substitution. Conservative amino acid substitutions are known in the art, and include amino acid substitutions in which one amino acid having certain physical and/or chemical properties is exchanged for another amino acid that has the same chemical or physical properties. For example, the conservative amino acid substitution can be an acidic amino acid substituted for another acidic amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Val, etc.), a basic amino acid substituted for another basic amino acid (Lys, Arg, etc.), an amino acid with a polar side chain substituted for another amino acid with a polar side chain (Asn, Cys, Gln, Ser, Thr, Tyr, etc.), etc.

Alternatively or additionally, the functional variants can comprise the amino acid sequence of the parent TCR with at least one non-conservative amino acid substitution. In this case, it is preferable for the non-conservative amino acid substitution not to interfere with or inhibit the biological activity of the functional variant. Preferably, the non-conservative amino acid substitution enhances the biological activity of the functional variant, such that the biological activity of the functional variant is increased as compared to the parent TCR, polypeptide, or protein.

The amino acid substitution(s) of the amino acid sequence of the functional variant can be within any region of the amino acid sequence. For example, in some embodiments, the amino acid substitution(s) is located within the region of the amino acid sequence which encodes the variable region or the constant region of the functional variant. In the instance that the amino acid substitution(s) is/are located within the region of the amino acid sequence which encodes the variable region (e.g., a Vβ CDR3 amino acid sequence, such as SEQ ID NO: 1), it is understood that the amino acid substitution(s) do not significantly decrease the ability of the functional variant to bind to the peptide-MHC complex for which the parent TCR has antigenic specificity.

Functional portions of the engineered TCRs are also provided. In some embodiments, the functional portions can comprise any portion comprising contiguous amino acids of the parent TCR, provided that the functional portion comprises a portion of the Vβ chain comprising the amino acid sequence set forth in SEQ ID NO: 1. The term “functional portion” when used in reference to a TCR refers to any part or fragment of the TCR of the invention, which part or fragment retains the biological activity of the TCR of which it is a part (the parent TCR). Functional portions encompass, for example, those parts of a TCR that retain the ability to, e.g., specifically bind to NY-ESO-1 peptide-MHC complex, as the parent TCR.

In some embodiments, the functional portion comprises additional amino acids at the amino- or carboxy-terminus of the portion, or at both termini, that do not interfere with the biological function of the TCR portion.

The TCRs (including functional portions and functional variants) described herein optionally comprise synthetic amino acids in place of one or more naturally-occurring amino acids. Such synthetic amino acids are known in the art, and include, for example, aminocyclohexane carboxylic acid, norleucine, .alpha.-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine .beta.-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine.

The TCRs (including functional portions and functional variants) described herein can be glycosylated, amidated, carboxylated, phosphorylated, esterified, N-acylated, cyclized via, e.g., a disulfide bridge, or converted into an acid addition salt and/or optionally dimerized or polymerized, or conjugated.

In some embodiments, it is desirable to identify the presence of a TCR comprising a CDR3 sequence described herein that is specific for NY-ESO-1/MHC complex. Methods for identifying the presence of the TCR include deep sequencing strategies such as IMMUNOSEQ™ which is commercially available from Adaptive Biotechnologies (Seattle, Wash.). See also Nature, 515, 568-571 (27 Nov. 2014); Carreno et al., 2015 Science 348:803-808). IMMUNOSEQ was used in Examples 2-7 to discover the TCR CDR3 sequences herein, but methods for detecting the presence of the CDR3 sequences described herein are not limited to this method. Any technology that detects the presence or absence of specific nucleotide sequences encoding the TCR CDR3 sequences are contemplated for use herein. Additionally, the public TCRs having the VβCDR3 amino acid sequences described herein might be detected directly by immunoassay with monoclonal antibodies developed for this purpose. The immunoassays which can be used include, but are not limited to, competitive assay systems using techniques such western blots, radioimmunoassays, ELISA, “sandwich” immunoassays, immunoprecipitation assays, precipitin assays, gel diffusion precipitin assays, immunoradiometric assays, fluorescent immunoassays, protein A, immunoassays, and complement-fixation assays. Such assays are routine and well known in the art (see, e.g., Ausubel et al, eds, 1994 Current Protocols in Molecular Biology, Vol. 1, John Wiley & sons, Inc., New York). Additionally, routine cross-blocking assays such as those described in Antibodies, A Laboratory Manual, Cold Spring Harbor Laboratory, Ed Harlow and David Lane, 1988), can be performed. Either nucleic acid based assays or protein based detection assays can be used to determine in an individual the presence or absence of T cells having TCRs comprising a VβCDR3 amino acid sequence described herein.

### Nucleic Acids, Vectors and Cells

Nucleic acids comprising a nucleotide sequence encoding any of the engineered TCRs (or functional portion and functional variant thereof) described herein are also contemplated.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

Preferably, the nucleic acids described herein are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acids can be constructed based on chemical synthesis and/or enzymatic ligation reactions using procedures known in the art or commercially available (e.g, from Genscript, Thermo Fisher and similar companies). See, for example, Sambrook et al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl)uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid can comprise any nucleotide sequence which encodes any of the engineered TCRs, polypeptides, or proteins, or functional portions or functional variants thereof.

The present disclosure also provides variants of the isolated or purified nucleic acids wherein the variant nucleic acids comprise a nucleotide sequence that has at least 75%, 80%, 81%, 82%, 83%, 84%, 85%, 86%, 87%, 88%, 89%, 90%, 91%, 92%, 93%, 94%, 95%, 96%, 97%, 98%, or 99% identical to the nucleotide sequence encoding the parent TCR. In certain embodiments, the present disclosure provides isolated or purified nucleic acids comprising a nucleotide sequence that is at least 75%, 80%, 81%, 82%, 83%, 84%, 85%, 86%, 87%, 88%, 89%, 90%, 91%, 92%, 93%, 94%, 95%, 96%, 97%, 98%, or 99% identical to a nucleotide sequence provided in the sequence listing herein, wherein such variant nucleotide sequence encodes a functional TCR that specifically recognizes its cognate MHC-peptide complex (e.g., NY-ESO-1 peptide/MHC complex) at least as well as the parent TCR.

The disclosure also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions preferably hybridizes under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the TCRs described herein. It is generally appreciated that conditions can be rendered more stringent by the addition of increasing amounts of formamide.

In certain embodiments, the nucleic acids described herein can be incorporated into any of a variety of different types of vectors. In this regard, the disclosure provides one or more recombinant expression vectors comprising any one or more of the nucleic acids described herein. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors described herein are not naturally-occurring as a whole. However, parts of the vectors can be naturally-occurring. The recombinant expression vectors described herein can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages does not hinder the transcription or replication of the vector.

The recombinant expression vector described herein can be any suitable recombinant expression vector, and can be used to transform, transfect or transduce any suitable host. Suitable vectors include those designed for propagation and expansion or for expression or both, such as plasmids and viruses. The vector can be selected from the group consisting of the pUC series (Fermentas Life Sciences), the pBluescript series (Stratagene, LaJolla, Calif.), the pET series (Novagen, Madison, Wis.), the pGEX series (Pharmacia Biotech, Uppsala, Sweden), and the pEX series (Clontech, Palo Alto, Calif.) and other commercially available plasmid vectors. Bacteriophage vectors, such as λ610, λGT11, λZapII (Stratagene), λEMBL4, and λNM1149, also can be used. Examples of plant expression vectors include pBI01, pBI101.2, pBI101.3, pBI121 and pBIN19 (Clontech). Examples of animal expression vectors include pEUK-C1, pMAM and pMAMneo (Clontech).

In some embodiments, a vector for use herein is a viral vector, e.g., a retroviral vector, such as a lentiviral vector, an adenoviral vector, a poxvirus vector, a vaccinia virus vector. “Lentivirus” refers to a genus of retroviruses that are capable of infecting dividing and non-dividing cells. Several examples of lentiviruses include HIV (human immunodeficiency virus: including HIV type 1, and HIV type 2); equine infectious anemia virus; feline immunodeficiency virus (FIV); bovine immune deficiency virus (BIV); and simian immunodeficiency virus (SIV).

Exemplary lentiviral vectors include, but are not limited to, vectors derived from HIV-1, HIV-2, FIV, equine infectious anemia virus, SIV, and maedi/visna virus. Methods of using viral vectors, retroviral and lentiviral viral vectors and packaging cells for transducing mammalian target cells with viral particles containing TCRs transgenes are well known in the art and have been previous described, for example, in U.S. Pat. No. 8,119,772; Walchli et al., 2011, PLoS One 6:327930; Zhao et al., J. Immunol., 2005, 174:4415-4423; Engels et al., 2003, Hum. Gene Ther. 14:1155-68; Frecha et al., 2010, Mol. Ther. 18:1748-57; Verhoeyen et al., 2009, Methods Mol. Biol. 506:97-114. Retroviral and lentiviral vector constructs and expression systems are also commercially available.

The recombinant expression vectors can be prepared using standard recombinant DNA techniques described in, for example, Current Protocols in Molecular Biology (2015 John Wiley & Sons, Inc) or Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, λ, SV40, bovine papilloma virus, and the like.

In some embodiments, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA- or RNA-based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the engineered TCR, polypeptide, or protein (including functional portions and functional variants thereof), or to the variant nucleotide sequence encoding a functional TCR, or to the nucleotide sequence which is complementary to or which hybridizes to the nucleotide sequence encoding the modified TCR, polypeptide, or protein. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, an EF1α promoter, a ubiquitin promoter, an MHC Class I or II promoter, a T cell specific promoter, a cytokine promoter, or a promoter found in the long-terminal repeat of the murine stem cell virus. In certain embodiments, the promoter is a synthetic promoter.

As discussed herein, in those embodiments where viral vectors are used, the viral vector genome comprises a sequence of interest that is desirable to express in target cells. With regard to retroviral vectors, typically, the sequence of interest (e.g., a nucleic acid encoding an engineered TCR as described herein) is located between the 5′ LTR and 3′ LTR sequences (or partial 5′ or 3′ LTR sequence as may be used in certain embodiments). In certain embodiments, the sequence of interest is in a functional relationship with other genetic elements, for example transcription regulatory sequences including promoters or enhancers, to regulate expression of the sequence of interest in a particular manner. In certain instances, the useful transcriptional regulatory sequences are those that are highly regulated with respect to activity, both temporally and spatially. Expression control elements that may be used for regulating the expression of the components are known in the art and include, but are not limited to, inducible promoters, constitutive promoters, secretion signals, enhancers and other regulatory elements.

The sequence of interest and any other expressible sequence is typically in a functional relationship with internal promoter/enhancer regulatory sequences. An “internal” promoter/enhancer is one that is located between the 5′ LTR and the 3′ LTR sequences (or partial sequences thereof) in the viral vector construct and is operably linked to the sequence of interest. The internal promoter/enhancer may be any promoter, enhancer or promoter/enhancer combination known to increase expression of a nucleic acid with which it is in a functional relationship. A “functional relationship” and “operably linked” mean, without limitation, that the sequence is in the correct location and orientation with respect to the promoter and/or enhancer that the sequence of interest will be expressed when the promoter and/or enhancer is contacted with the appropriate molecules.

The choice of an internal promoter/enhancer is based on the desired expression pattern of the sequence of interest and the specific properties of known promoters/enhancers. Thus, the internal promoter may be constitutively active. Non-limiting examples of constitutive promoters that may be used include the promoter for ubiquitin (U.S. Pat. No. 5,510,474; WO 98/32869, each of which is incorporated herein by reference in its entirety), CMV (Thomsen et al., PNAS 81:659, 1984; U.S. Pat. No. 5,168,062, each of which is incorporated herein by reference in its entirety), beta-actin (Gunning et al. 1989 Proc. Natl. Acad. Sci. USA 84:4831-4835, which is incorporated herein by reference in its entirety) and pgk (see, for example, Adra et al. 1987 Gene 60:65-74; Singer-Sam et al. 1984 Gene 32:409-417; and Dobson et al. 1982 Nucleic Acids Res. 10:2635-2637, each of the foregoing which is incorporated herein by reference in its entirety). In some embodiments, the promoter used to control expression of the sequence of interest (e.g., the engineered TCRs described herein) encoded by a vector (e.g., a pseudotyped retroviral vector genome) is an intron-deficient promoter. In some embodiments, the human Ubiquitin-C(UbiC) promoter is used to control expression of the TCRs encoded by the viral vector genome. In various embodiments, the UbiC promoter has been modified to remove introns, i.e., the promoter is intron deficient. The full-length UbiC promoter is 1250 nucleotides. The intron begins at 412 and goes all the way to the end (412-1250). This region can be deleted for the purpose of minimizing heterogeneous viral genomic transcripts. The HIV viral genome has a native intron within it. Thus, a lentivirus comprising a UbiC promoter would have a total of 2 introns in the lentivirus genome. The UbiC intron can exist in both spliced and unspliced forms. Deletion of the UbiC intron eliminates the possibility of heterogenous viral transcripts and ensures homogeneity in the delivered pseudotyped lentiviral particles.

Alternatively, the promoter may be a tissue specific promoter. In some preferred embodiments, the promoter is a target cell-specific promoter. For example, the promoter can be from any product expressed by dendritic cells, T cells, NK cells, including but not limited to, IL-2, IL-2R, interferon γ, MHC class I, MHC class II, CD3, CD11c, CD103, TLRs, DC-SIGN, BDCA-3, DEC-205, DCIR2, mannose receptor, Dectin-1, Clec9A. In addition, promoters may be selected to allow for inducible expression of the sequence of interest. A number of systems for inducible expression are known in the art, including the tetracycline responsive system, the lac operator-repressor system, as well as promoters responsive to a variety of environmental or physiological changes, including heat shock, metal ions, such as metallothionein promoter, interferons, hypoxia, steroids, such as progesterone or glucocorticoid receptor promoter, radiation, such as VEGF promoter. A combination of promoters may also be used to obtain the desired expression of the gene of interest. The artisan of ordinary skill will be able to select a promoter based on the desired expression pattern of the gene in the organism or the target cell of interest.

The viral genome may comprise at least one RNA Polymerase II or III responsive promoter. This promoter can be operably linked to the sequence of interest and can also be linked to a termination sequence. In addition, more than one RNA Polymerase II or III promoters may be incorporated. RNA polymerase II and III promoters are well known to one of skill in the art. A suitable range of RNA polymerase III promoters can be found, for example, in Paule and White, Nucleic Acids Research, Vol. 28, pp 1283-1298 (2000), which is incorporated herein by reference in its entirety. RNA polymerase II or III promoters also include any synthetic or engineered DNA fragment that can direct RNA polymerase II or III to transcribe downstream RNA coding sequences. Further, the RNA polymerase II or III (Pol II or III) promoter or promoters used as part of the viral vector genome can be inducible. Any suitable inducible Pol II or III promoter can be used with the methods of the disclosure. Particularly suited Pol II or III promoters include the tetracycline responsive promoters provided in Ohkawa and Taira, Human Gene Therapy, Vol. 11, pp 577-585 (2000) and in Meissner et al. Nucleic Acids Research, Vol. 29, pp 1672-1682 (2001), each of which is incorporated herein by reference in its entirety.

An internal enhancer may also be present in the viral construct to increase expression of the gene of interest. For example, the CMV enhancer (Boshart et al. Cell, 41:521, 1985; which is incorporated herein by reference in its entirety) may be used. Many enhancers in viral genomes, such as HIV, CMV, and in mammalian genomes have been identified and characterized (see GenBank). An enhancer can be used in combination with a heterologous promoter. One of ordinary skill in the art will be able to select the appropriate enhancer based on the desired expression pattern.

The viral vector genome may also contain additional genetic elements. The types of elements that may be included in the construct are not limited in any way and may be chosen to achieve a particular result. For example, a signal that facilitates nuclear entry of the viral genome in the target cell may be included. An example of such a signal is the HIV-1 cPPT/CTS. Further, elements may be included that facilitate the characterization of the provirus integration site in the target cell. For example, a tRNA amber suppressor sequence may be included in the construct. An insulator sequence from e.g., chicken β-globin may also be included in the viral genome construct. This element reduces the chance of silencing an integrated provirus in the target cell due to methylation and heterochromatinization effects. In addition, the insulator may shield the internal enhancer, promoter and exogenous gene from positive or negative positional effects from surrounding DNA at the integration site on the chromosome. In addition, the vector genome may contain one or more genetic elements designed to enhance expression of the gene of interest. For example, a woodchuck hepatitis virus responsive element (WRE) may be placed into the construct (Zufferey et al. 1999. J. Virol. 74:3668-3681; Deglon et al. 2000. Hum. Gene Ther. 11:179-190, each of which is incorporated herein by reference in its entirety).

The viral vector genome is typically constructed in a plasmid form that may be transfected into a packaging or producer cell line. The plasmid generally comprises sequences useful for replication of the plasmid in bacteria. Such plasmids are well known in the art. In addition, vectors that include a prokaryotic origin of replication may also include a gene whose expression confers a detectable or selectable marker such as a drug resistance. Typical bacterial drug resistance products are those that confer resistance to ampicillin or tetracycline.

The inventive recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

Also provided is a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, 293F, 293T cells, and the like. In particular, for the purposes of producing viral particles for delivery of the TCRs described herein, 293F and 293T host cells may be used. For purposes of amplifying or replicating the recombinant expression vector, the host cell may be a prokaryotic cell, e.g., a DH5a cell. For purposes of producing a recombinant modified TCR, polypeptide, or protein, the host cell may be a mammalian cell. In certain embodiments, the host cell is a human cell. The host cell can be of any cell type, can originate from any type of tissue, and can be of any developmental stage. In certain embodiments, the host cell is a peripheral blood lymphocyte (PBL). In certain embodiments, the host cell is a T cell.

In some embodiments, a vector described herein encodes a TCR (or functional variant or portion) described herein. In this regard, in those embodiments where the TCR is a heterodimeric TCR, both the TCR beta chain and the TCR alpha chain may be expressed from the same vector or may be expressed from different vectors within the same host cell such that a functional dimeric TCR is expressed at the surface of the cell. In other embodiments where the TCR is a single chain TCR, a vector described herein comprises a nucleic acid encoding the single chain TCR or other forms of the TCRs described herein.

In additional embodiments, a vector described herein may encode more than one product. In this regard, the sequence to be delivered can comprise a nucleic acid encoding a TCR as described herein in addition to other nucleic acids of interest, encoding multiple genes encoding at least one protein, at least one siRNA, at least one microRNA, at least one dsRNA or at least one anti-sense RNA molecule or any combinations thereof. For example, the sequence to be delivered can include one or more genes that encode one or more TCRs. The one or more TCRs can be associated with a single disease or disorder, or they can be associated with multiple diseases and/or disorders. In some instances, a gene encoding an immune regulatory protein can be included along with a gene encoding a TCR as described herein, and the combination can elicit and regulate the immune response to the desired direction and magnitude. In other instances, a sequence encoding an siRNA, microRNA, dsRNA or anti-sense RNA molecule can be constructed with a gene encoding a TCR as described herein, and the combination can regulate the scope of the immune response. The products may be produced as an initial fusion product in which the encoding sequence is in functional relationship with one promoter. Alternatively, the products may be separately encoded and each encoding sequence in functional relationship with a promoter. The promoters may be the same or different.

In some embodiments, vectors contain polynucleotide sequences that encode immunomodulatory molecules. Exemplary immunomodulatory molecules include GM-CSF, IL-2, IL-4, IL-6, IL-7, IL-12, IL-15, IL-18 IL-21, IL-23, interferon gamma, TNFα, B7.1, B7.2, 4-1BB, CD40, CD40 ligand (CD40L), drug-inducible CD40 (iCD40), and the like, or ligands, or single chain antibodies that bind thereto. These polynucleotides are typically under the control of one or more regulatory elements that direct the expression of the coding sequences in host cells.

In certain embodiments, the vectors described herein may express a checkpoint inhibitor. The checkpoint inhibitor may be expressed from the same vector as the TCRs described herein or from a separate vector. Immune checkpoints refer to a variety of inhibitory pathways of the immune system that are crucial for maintaining self-tolerance and for modulating the duration and amplitude of an immune responses. Tumors use certain immune-checkpoint pathways as a major mechanism of immune resistance, particularly against T cells that are specific for tumor antigens. (see, e.g., Pardo11, 2012 Nature 12:252; Chen and Mellman 2013 Immunity 39:1). The present disclosure provides immune checkpoint inhibitors that can be expressed from the expression vectors described herein in combination with the TCRs described herein. Illustrative checkpoint inhibitors include antibodies, or antigen-binding fragments thereof, that bind to and block or inhibit immune checkpoint receptors or antibodies, or antigen-binding fragments thereof that bind to and block or inhibit immune checkpoint receptor ligands. Illustrative immune checkpoint molecules that may be targeted for blocking or inhibition include, but are not limited to, CTLA-4, 4-1BB (CD137), 4-1BBL (CD137L), PDL1, PDL2, PD1, B7-H3, B7-H4, BTLA, HVEM, TIM3, GAL9, LAG3, TIM3, B7H3, B7H4, VISTA, KIR, 2B4 (belongs to the CD2 family of molecules and is expressed on all NK, γδ, and memory CD8⁺ (4) T cells), CD160 (also referred to as BY55) and CGEN-15049. Immune checkpoint inhibitors include antibodies, or antigen binding fragments thereof, or other binding proteins, that bind to and block or inhibit the activity of one or more of CTLA-4, PDL1, PDL2, PD1, B7-H3, B7-H4, BTLA, HVEM, TIM3, GAL9, LAG3, TIM3, B7H3, B7H4, VISTA, KIR, 2B4, CD160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody) and Yervoy/ipilimumab (anti-CTLA-4 checkpoint inhibitor).

The expression vectors (e.g., retroviral vectors or lentiviral vectors) for expressing the TCRs herein can be engineered to express more than one, e.g., two, three, or four, sequences of interest at a time. Several methods are known in the art for simultaneously expressing more than one sequences from a single vector. For example, the vectors can comprise multiple promoters fused to a coding sequence's open reading frames (ORFs), insertion of splicing signals between coding sequences, fusion of sequences of interest whose expressions are driven by a single promoter, insertion of proteolytic cleavage sites between coding sequences, insertion of internal ribosomal entry sites (IRESs) between coding sequences, insertion of bi-directional promoters between coding sequences, and/or “self-cleaving” 2A peptides. Each component to be expressed in a multicistronic expression vector may be separated, for example, by an internal ribosome entry site (IRES) element or a viral 2A element, to allow for separate expression of the various proteins from the same promoter. IRES elements and 2A elements are known in the art (U.S. Pat. No. 4,937,190; de Felipe et al. 2004. Traffic 5: 616-626, each of which is incorporated herein by reference in its entirety). In one embodiment, oligonucleotides encoding furin cleavage site sequences (RAKR) (Fang et al. 2005. Nat. Biotech 23: 584-590, which is incorporated herein by reference in its entirety) linked with 2A-like sequences from foot-and-mouth diseases virus (FMDV; F2A), porcine teschovirus-1 (P2A), equine rhinitis A virus (ERAV; E2A), and thosea asigna virus (TaV; T2A) (Szymczak et al. 2004. Nat. Biotechnol. 22: 589-594, which is incorporated herein by reference in its entirety) are used to separate genetic elements in a multicistronic vector. The efficacy of a particular multicistronic vector can readily be tested by detecting expression of each of the genes using standard protocols.

Expression of two or more sequences of interest (e.g. sequences encoding a TCR alpha chain and a TCR beta chain; a single chain TCR and an immunomodulatory molecule) can also be accomplished using Internal Ribosome Entry Sites (IRES). IRES enable eukaryotic ribosomes to enter and scan an mRNA at a position other than the 5′ m⁷ G-cap structure. If positioned internally, e.g., 3′ of a first coding region (or cistron), an IRES will enable translation of a second coding region within the same transcript. The second coding region is identified by the first ATG encountered after the IRES. Exemplary IRES elements include viral IRES such as the picornavirus IRES and the cardiovirus IRES (see, e.g., U.S. Pat. No. 4,937,190) and non-viral IRES elements found in 5′ UTRs (e.g., those elements of transcripts encoding immunoglobulin heavy chain binding protein (BiP) (Macejak et al., Nature, 35390-4, 1991); Drosophila Antennapedia (Oh et al., Genes Dev. 6:1643-53, 1992) and Ultrabithorax (Ye et al., Mol. Cell Biol., 17:1714-21, 1997); fibroblast growth factor 2 (Vagner et al., Mol. Cell Biol., 15:35-44, 1995); initiation factor eIF4G (Gan et al., J. Biol. Chem. 273:5006-12, 1998); proto-oncogene c-myc (Nanbru et al., J. Biol. Chem., 272:32061-6, 1995; Stoneley, Oncogene, 16:423-8, 1998); and vascular endothelial growth factor (VEGF) (Stein et al., Mol. Cell Biol., 18:3112-9, 1998).

Expression of two or more sequences of interest can also be accomplished using bidirectional promoters, i.e., a promoter region or two back-to-back cloned promoters whose reading directions point away from each other, and from which two open reading frames flanking the promoter region are transcribed. Examples of such promoters include the PDGF-A, neurotropic JC virus, BRCA1, transcobalamin II, and dipeptidylpeptidase IV promoters.

### Production of Lentiviral Particles

In certain embodiments, retroviral vectors are used to transduce T cells to modify the T cells to express the TCRs of the present invention, and other sequences of interest as described herein. Any of a variety of methods already known in the art may be used to produce infectious viral, e.g., retroviral and lentiviral, particles whose genome comprises an RNA copy of the viral vector genome. In one method, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences of interest. In order to prevent replication of the genome in the target cell, however, endogenous viral genes required for replication will usually be removed and provided separately in the packaging cell line.

In general, the retroviral vector particles are produced by a cell line that is transfected with one or more plasmid vectors containing the components necessary to generate the particles. These retroviral vector particles are typically not replication-competent, i.e., they are only capable of a single round of infection. Most often, multiple plasmid vectors are utilized to separate the various genetic components that generate the vector particles, mainly to reduce the chance of recombination events that might otherwise generate replication competent viruses. A single plasmid vector having all of the retroviral components can be used if desired, however. As one example of a system that employs multiple plasmid vectors, a cell line is transfected with at least one plasmid containing the viral vector genome (i.e., the vector genome plasmid), including the LTRs, the cis-acting packaging sequence, and the sequence(s) of interest, which are often operably linked to a heterologous promoter, at least one plasmid encoding the virus enzymatic and structural components (i.e., the packaging plasmid that encodes components such as, Gag and Pol), and at least one envelope plasmid encoding an envelope glycoprotein (e.g., an envelope protein derived from a retrovirus or other suitable envelope glycoproteins such as VSV G, Sindbis envelope, measles virus envelope, and the like). Additional plasmids can be used to enhance retrovirus particle production, e.g., Rev-expression plasmids, as described herein and known in the art. Viral particles bud through the cell membrane and comprise a core that includes a genome containing the sequence of interest and an envelope glycoprotein.

Transfection of packaging cells with plasmid vectors of the present disclosure can be accomplished by well-known methods, and the method to be used is not limited in any way. A number of non-viral delivery systems are known in the art, including for example, electroporation, lipid-based delivery systems including liposomes, delivery of “naked” DNA, and delivery using polycyclodextrin compounds, such as those described in Schatzlein A G. (2001. Non-Viral Vectors in Cancer Gene Therapy: Principles and Progresses. Anticancer Drugs, which is incorporated herein by reference in its entirety). Cationic lipid or salt treatment methods are typically employed, see, for example, Graham et al. (1973. Virol. 52:456; Wigler et al. (1979. Proc. Natl. Acad. Sci. USA 76:1373-76), each of the foregoing which is incorporated herein by reference in its entirety. The calcium phosphate precipitation method is most often used. However, other methods for introducing the vector into cells may also be used, including nuclear microinjection and bacterial protoplast fusion.

The packaging cell line provides the components, including viral regulatory and structural proteins, that are required in trans for the packaging of the viral genomic RNA into retroviral (e.g., lentiviral) vector particles. The packaging cell line may be any cell line that is capable of expressing lentiviral proteins and producing functional lentiviral vector particles. Some suitable packaging cell lines include 293 (ATCC CCL X), 293T, HeLa (ATCC CCL 2), D17 (ATCC CCL 183), MDCK (ATCC CCL 34), BHK (ATCC CCL-10) and Cf2Th (ATCC CRL 1430) cells. The packaging cell line may stably express the necessary viral proteins. Such a packaging cell line is described, for example, in U.S. Pat. No. 6,218,181, which is incorporated herein by reference in its entirety. Alternatively a packaging cell line may be transiently transfected with nucleic acid molecules encoding one or more necessary viral proteins along with the viral vector genome. The resulting viral particles are collected and used to infect a target cell. The gene(s) encoding envelope glycoprotein(s) is usually cloned into an expression vector, such as pcDNA3 (Invitrogen, Calif. USA). Eukaryotic cell expression vectors are well known in the art and are available from a number of commercial sources. Packaging cells, such as 293T cells are then co-transfected with the viral vector genome encoding a sequence of interest (typically encoding an antigen), at least one plasmid encoding virus packing components, and a vector for expression of the targeting molecule. The envelope is expressed on the membrane of the packaging cell and incorporated into the viral vector.

For purposes provided herein, the TCRs herein can be expressed in T cells or NK cells or other suitable cells of the immune system. The T cell can be any T cell, such as a cultured T cell, e.g., a primary T cell, or a T cell from a cultured T cell line, e.g., Jurkat, SupT1, etc., or a T cell obtained from a mammal. If obtained from a mammal, the T cell can be obtained from numerous sources, including but not limited to blood, peripheral blood mononuclear cells (PBMCs), peripheral blood leukocytes (PBLs), apheresis sample, bone marrow, lymph node, the thymus, or other tissues or fluids. T cells can also be enriched for or purified. In some embodiments, the T cell is a human T cell. In some embodiments, the T cell is a T cell isolated from a human. The T cell can be any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells (TILs), memory T cells, naïve T cells, and the like.

Also provided by the disclosure is a population of cells comprising at least one cell described herein. The population of cells can be a heterogeneous population comprising the host cell comprising any of the recombinant expression vectors described, in addition to at least one other cell, (e.g., a T cell), which does not comprise any of the recombinant expression vectors, or a cell other than a T cell, e.g., a B cell, NK cells, a macrophage, a neutrophil, an erythrocyte, a hepatocyte, an endothelial cell, an epithelial cells, a muscle cell, a brain cell, etc. Alternatively, the population of cells can be a substantially homogeneous population, in which the population comprises mainly of cells (e.g., consisting essentially of) comprising the recombinant expression vector. The population also can be a clonal population of cells, in which all cells of the population are clones of a single cell comprising a recombinant expression vector, such that all cells of the population comprise the recombinant expression vector. In one embodiment of the invention, the population of cells is a clonal population comprising cells comprising a recombinant expression vector as described herein.

Ex Vivo Genetic Modification of T Cells

As noted above, in certain embodiments, it may be desirable to use the viral vectors disclosed herein to genetically modify T cells ex vivo. In this regard, the sources of T cells, culture and expansion of the T cells is described.

Prior to expansion and genetic modification of the T cells, a source of T cells is obtained from a subject. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In certain embodiments of the present invention, any number of T cell lines available in the art, may be used. In certain embodiments of the present invention, T cells can be obtained from a unit of blood collected from a subject using any number of techniques known to the skilled artisan, such as FICOLL™ separation. In one embodiment, cells from the circulating blood of an individual are obtained by apheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Again, surprisingly, initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca²⁺⁻free, Mg²⁺-free PBS, PlasmaLyte A, or other saline solution with or without buffer. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient or by counterflow centrifugal elutriation. A specific subpopulation of T cells, such as CD3+, CD28+, CD4+, CD8+, CD45RA+, and CD45RO+ T cells, can be further isolated by positive or negative selection techniques. For example, in one embodiment, T cells are isolated by incubation with anti-CD3/anti-CD28 (i.e., 3×28)-conjugated beads, such as DYNABEADS™ M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immune-compromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain embodiments, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD 14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-C25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In certain embodiments, specific sub-types of T cells may be isolated and genetically modified with the lentiviral vector particles as described herein, using methods such as those described for example, in WO 2012/129514, the disclosure of which is incorporated by reference in its entirety.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

In other embodiments, the cells may be incubated on a rotator for varying lengths of time at varying speeds at either 2-10° C. or at room temperature.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80 C. at a rate of 1o per minute and stored in the vapor phase of a liquid nitrogen storage tank. Other methods of controlled freezing may be used as well as uncontrolled freezing immediately at −20 C. or in liquid nitrogen.

In certain embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or an apheresis is taken from a generally healthy subject. In certain embodiments, a blood sample or an apheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin) (Liu et al., Cell 66:807-815, 1991; Henderson et al., Immun 73:316-321, 1991; Bierer et al., Curr. Opin. Immun. 5:763-773, 1993). In a further embodiment, the cells are isolated for a patient and frozen for later use in conjunction with (e.g., before, simultaneously or following) bone marrow or stem cell transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g., Rituxan.

In a further embodiment, T cells are obtained from a patient directly following treatment. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

Whether prior to or after genetic modification of the T cells ex vivo to express a sequence of interest, the T cells can be activated and expanded generally using methods known in the art. Illustrative methods for activating and expanding T cells are as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and published application Nos. WO2012/129514 and US20060121005, the disclosure of which are incorporated herein by reference in their entireties.

In certain embodiments, T cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol. Meth. 227(1-2):53-63, 1999).

In certain embodiments, the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts.

In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation.

Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-.gamma., IL-4, IL-7, GM-CSF, IL-10, IL-12, IL-15, TGF.beta., and TNF-.alpha. or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (Tc, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

In certain embodiments, the present disclosure contemplates the use of T cells genetically modified to stably express a TCR as described herein. T cells expressing and engineered TCR, are referred to herein as chimeric TCR modified T cells. Preferably, the cell can be genetically modified to stably express an antibody binding domain on its surface, conferring novel antigen specificity that is MHC independent.

### Compositions Comprising and Administration of Modified T Cells

In certain embodiments, the present disclosure provides compositions comprising T cells that have been modified using the lentiviral vector particles described herein to express a transgene of interest, such as the engineered TCRs described herein. Such compositions can be administered to subjects in the methods of the present disclosure as described further herein.

Compositions comprising the modified T cells as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective amount of cells in the composition is typically greater than 10² cells, and up to 10⁶, up to and including 10⁸ or 10⁹ cells and can be more than 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 mls or less, even 250 mls or 100 mls or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10″ cells or the appropriate number of immune cells as determined by a clinician skilled in the art.

### Methods of Diagnosis and Treatment

Also provided herein are compositions comprising engineered TCRs (e.g., soluble TCRs described herein, and fusion proteins or chimeric proteins thereof); compositions comprising viral vector particles comprising a sequence encoding an engineered TCR; or compositions comprising cells, in particular T cells, expressing the engineered TCRs described herein for use in methods of treating cancer (e.g., a NY-ESO-1 cancer) or for use in inhibiting proliferation of a cancer cell that expresses NY-ESO-1.

In this regard, described herein is a method of treating a cancer associated with NY-ESO-1 expression in a mammalian subject comprising administering to the subject a therapeutic composition, said composition comprising one or more therapeutic agents selected from the group consisting of (a) an engineered TCR as described herein; (b) an isolated cell comprising a polynucleotide encoding an engineered TCR polypeptide disclosed herein; (c) a soluble TCR, or a chimeric or fusion polypeptide comprising the soluble TCR that is specific for NY-ESO-1 in the context of a MHC molecule; (d) a polynucleotide encoding an engineered TCR polypeptide; (e) a polynucleotide encoding a soluble TCR that is specific for NY-ESO-1 in the context of a MHC molecule; (f) a viral vector comprising a polynucleotide encoding an engineered TCR polypeptide herein that is specific for NY-ESO-1/MHC complex; wherein the therapeutic composition is administered to the subject in an amount effective to treat the cancer in the subject. Illustrative TCR sequences for use in the engineered TCRs described herein for use in the methods of treatment and methods for inhibiting the proliferation of cancer described herein are provided in the sequence listing and include the beta chain variable region provided in SEQ ID N0:9 and the alpha chain variable region provided in SEQ ID N0:8.

In this regard, described herein is a method of treating a cancer associated with NY-ESO-1 expression in a mammalian subject comprising administering to the subject a therapeutic composition, said composition comprising one or more therapeutic agents selected from the group consisting of (a) an isolated cell comprising a polynucleotide encoding an engineered TCR polypeptide comprising a VβCDR3 that is specific for NY-ESO-1, wherein the Vβ CDR3 comprises the amino acid sequence of CASSLNRDXXXXF (SEQ ID NO: 1) or wherein the V beta CDR3 comprises an amino acid sequence as set forth in SEQ ID NOs: 2-4; or where in the beta chain variable region is as provided in SEQ ID NO:9 and the alpha chain variable region is as provided in SEQ ID NO:8; (b) a soluble TCR, or a chimeric or fusion polypeptide comprising the soluble TCR, comprising a Vβ chain CDR3 that is specific for NY-ESO-1 in the context of a MHC molecule, wherein the Vβ CDR3 comprises the amino acid sequence of CASSLNRDXXXXF (SEQ ID NO: 1) or wherein the V beta CDR3 comprises an amino acid sequence as set forth in SEQ ID Nos: 2-4; (c) a polynucleotide encoding a chimeric TCR polypeptide comprising a Vβ chain CDR3 that is specific for NY-ESO-1, wherein the Vβ CDR3 comprises the amino acid sequence of CASSLNRDXXXXF (SEQ ID NO: 1) or wherein the V beta CDR3 comprises the amino acid sequence as set forth in SEQ ID Nos: 2-4; (d) a polynucleotide encoding a soluble TCR comprising a Vβ chain CDR3 that is specific for NY-ESO-1 in the context of a MHC molecule, wherein the Vβ CDR3 comprises the amino acid sequence of CASSLNRDXXXXF (SEQ ID NO: 1 or wherein the V beta CDR3 comprises an amino acid sequence as set forth in SEQ ID Nos: 2-4; (e) a vector comprising a polynucleotide encoding a chimeric TCR polypeptide comprising a Vβ chain CDR3 that is specific for NY-ESO-1; and (f) a vector comprising a Vβ chain CDR3 that is specific for NY-ESO-1 in the context of a MHC molecule, wherein the therapeutic composition is administered to the subject in an amount effective to treat the cancer in the subject. In certain of the embodiments described herein, the engineered TCR comprises the alpha chain variable region and the beta chain variable region as provided in SEQ ID NO:8 and 9 respectively.

The terms “NY-ESO-1 cancer” and “cancer cell that expresses NY-ESO-1” as used herein refer to a tumor comprising cells that express the NY-ESO-1 tumor antigen. Such cancers are known in the art and expression of NY-ESO-1 in a particular cancer can be determined by a person of ordinary skill in the art. In some embodiments, the tumor is a solid tumor. Exemplary NY-ESO-1 cancers include, but are not limited to, sarcoma (e.g. soft tissue sarcoma), melanoma, lymphoma, prostate cancer, uterine cancer, thyroid cancer, testicular cancer, renal cancer, pancreatic cancer, ovarian cancer, oesophageal cancer, non-small-cell lung cancer, non-Hodgkin's lymphoma, NHL (DLCL), multiple myeloma, hepatocellular carcinoma, head and neck cancer, gastric cancer, endometrial cancer, renal cancer, colorectal cancer, cholangiocarcinoma, breast cancer, bladder cancer, neuroblastoma, myeloid leukemia and acute lymphoblastic leukemia.

Also described herein is a method of inhibiting proliferation of a cancer cell that expresses NY-ESO-1 in a mammalian subject comprising administering to the subject a therapeutic composition comprising one or more therapeutic agents selected from the group consisting of (a) an isolated cell comprising a polynucleotide encoding a chimeric TCR polypeptide comprising a Vβ chain complementarity determining region 3 (CDR3) that is specific for NY-ESO-1, wherein the Vβ CDR3 comprises the amino acid sequence of CASSLNRDXXXXF (SEQ ID NO: 1) or wherein the V beta CDR3 comprises an amino acid sequence as set forth in SEQ ID Nos: 2-4; (b) a soluble TCR comprising a Vβ chain CDR3 that is specific for NY-ESO-1 in the context of a MHC molecule, wherein the Vβ CDR3 comprises the amino acid sequence of CASSLNRDXXXXF (SEQ ID NO: 1) or wherein the V beta CDR3 comprises an amino acid sequence as set forth in SEQ ID Nos: 2-4; (c) a polynucleotide encoding a chimeric TCR polypeptide comprising a Vβ chain CDR3 that is specific for NY-ESO-1, wherein the Vβ CDR3 comprises the amino acid sequence of CASSLNRDXXXXF (SEQ ID NO: 1) or wherein the V beta CDR3 comprises an amino acid sequence as set forth in SEQ ID Nos: 2-4; (d) a polynucleotide encoding a soluble TCR comprising a Vβ chain CDR3 that is specific for NY-ESO-1 in the context of a MHC molecule, wherein the Vβ CDR3 comprises the amino acid sequence of CASSLNRDXXXXF (SEQ ID NO: 1) or wherein the V beta CDR3 comprises an amino acid sequence as set forth in SEQ ID Nos: 2-4; (e) a vector comprising a polynucleotide encoding a chimeric TCR polypeptide comprising a Vβ chain CDR3 that is specific for NY-ESO-1; and (f) a vector comprising a Vβ chain CDR3 that is specific for NY-ESO-1 in the context of a MHC molecule, wherein the therapeutic composition is administered to the subject in an amount effective to inhibit proliferation of the cancer cell in the subject. In certain of the embodiments described herein, the engineered TCR comprises the alpha chain variable region and the beta chain variable region as provided in SEQ ID NO:8 and 9 respectively.

A “therapeutically effective amount” or “effective amount” as used herein, means an amount which provides a therapeutic or prophylactic benefit.

Methods of identifying subjects likely to benefit from treatment with a therapeutic composition, such as the compositions described herein are also contemplated. In this regard, the method comprises (a) identifying a mammalian subject as likely to benefit from a NY-ESO-1 cancer therapy comprising determining in a sample from the mammalian subject the presence of (i) a polynucleotide encoding a TCR polypeptide comprising a Vβ chain CDR3 that is specific for NY-ESO-1, wherein the Vβ chain comprises the amino acid sequence of CASSLNRDXXXXF (SEQ ID NO: 1) or wherein the V beta CDR3 comprises an amino acid sequence as set forth in SEQ ID Nos: 2-4; or (ii) a TCR polypeptide comprising a Vβ chain CDR3 that is specific for NY-ESO-1, wherein the Vβ chain comprises the amino acid sequence of CASSLNRDXXXXF (SEQ ID NO: 1) or wherein the V beta CDR3 comprises an amino acid sequence as set forth in SEQ ID Nos: 2-4; wherein the presence of (i) and/or (ii) is indicative that the subject will likely benefit from a NY-ESO-1 cancer therapy.

In a further embodiment, a method of treating a subject that has been identified as a subject that is likely to benefit from the treatment is also contemplated herein. In this regard, the method of treatment comprises (a) identifying a mammalian subject as likely to benefit from a NY-ESO-1 cancer therapy comprising determining in a sample from the mammalian subject the presence of (i) a polynucleotide encoding a TCR polypeptide comprising a Vβ chain CDR3 that is specific for NY-ESO-1, wherein the Vβ chain comprises the amino acid sequence of CASSLNRDXXXXF (SEQ ID NO: 1) or wherein the V beta CDR3 comprises an amino acid sequence as set forth in SEQ ID Nos: 2-4; or (ii) a TCR polypeptide comprising a Vβ chain CDR3 that is specific for NY-ESO-1, wherein the Vβ chain comprises the amino acid sequence of CASSLNRDXXXXF (SEQ ID NO: 1) or wherein the V beta CDR3 comprises an amino acid sequence as set forth in SEQ ID Nos: 2-4; wherein the presence of (i) and/or (ii) is indicative that the subject will likely benefit from a NY-ESO-1 cancer therapy; and (b) administering the NY-ESO-1 cancer therapy to the mammalian subject.

The presence of a polynucleotide encoding a TCR polypeptide comprising a Vβ chain CDR3 that is specific for NY-ESO-1, wherein the Vβ chain comprises the amino acid sequence of CASSLNRDXXXXF (SEQ ID NO: 1) or wherein the V beta CDR3 comprises an amino acid sequence as set forth in SEQ ID Nos: 2-4 can be determined, for example, by the deep sequencing methods such as those commercially available from Adaptive Biotechnologies (Seattle, Wash.) and described in Examples 2-7. Other methods, including multiplex PCR, and other technologies known in the art that detect the presence or absence of specific nucleotide sequences can also be utilized. Additionally, the TCR polypeptides may be detected directly by immunoassay with either the appropriate tetramer or monoclonal antibodies developed for this purpose. The immunoassays which can be used include, but are not limited to, competitive assay systems using techniques such as western blots, radioimmunoassays, ELISA, “sandwich” immunoassays, immunoprecipitation assays, precipitin assays, gel diffusion precipitin assays, immunoradiometric assays, fluorescent immunoassays, protein A, immunoassays, plasmon surface residence, and complement-fixation assays, and the like. Such assays are routine and well known in the art (see, e.g., Ausubel et al, eds, 2015 Current Protocols in Molecular Biology, Vol. 1, John Wiley & sons, Inc., New York). Additionally, routine cross-blocking assays such as those described in Antibodies, A Laboratory Manual, Cold Spring Harbor Laboratory, Ed Harlow and David Lane, 1988), can be performed. Either nucleic acid based or protein based assays can distinguish individuals carrying a TCR comprising a Vβ chain having the CDR3 amino acid sequence described herein at high frequency from individuals not carrying the diagnostic clonotype.

The methods of treatment contemplated herein include any NY-ESO-1 specific cancer therapy, in particular immuno therapies. In one embodiment, the treatment methods useful for patients expressing the public TCRs as described herein are such as those described in U.S. Pat. No. 9,044,420. In some embodiments, the NY-ESO-1 cancer therapy comprises administering a vector comprising a polynucleotide encoding an NY-ESO-1 polypeptide to the subject. In some embodiments, the vector is a lentiviral vector. In some embodiments, the vector comprises a polynucleotide encoding an NY-ESO-1 polypeptide. In some embodiments, the vector comprises a polynucleotide encoding an NY-ESO-1 polypeptide.

In some embodiments, the NY-ESO-1 cancer therapy comprises administering to the subject an effective amount of a composition comprising GLA, said composition comprising:

(a) GLA of formula (I):

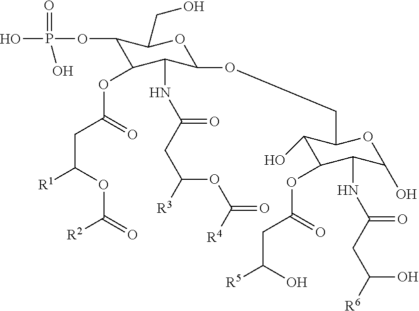

wherein: R¹, R³, R⁵ and R⁶ are C₁₁-C₂₀ alkyl; and R² and R⁴ are C₁₂-C₂₀ alkyl; and

(b) a pharmaceutically acceptable carrier or excipient; wherein the composition does not comprise antigen. In one embodiments of the methods described herein, R¹, R³, R⁵ and R⁶ are undecyl and R² and R⁴ are tridecyl. In another embodiment of the methods described herein, the mammal is human. In yet a further embodiment, the composition is an aqueous formulation, and in certain embodiments, the composition is in the form of an oil-in-water emulsion, a water-in-oil emulsion, liposome, micellar formulation, or a microparticle.

U.S. Patent Publication No. 2008/0131466 that provides formulations, such as aqueous formulation (AF) and stable emulsion formulations (SE) for GLA compounds, wherein these formulations may be used for any of the compounds of formula (I).

GLA as described herein is present in a composition in an amount of 0.1-10 μg/dose, or 0.1-20 μg/dose, 0.1-30 μg/dose, 0.1-40 μg/dose, or 0.1-50 μg/dose, or 1-20 μg/dose, or 1-30 μg/dose, or 1-40 μg/dose, or 1-50 μg/dose, or 0.2-5 μg/dose, or in an amount of 0.5-2.5 μg/dose, or in an amount of 0.5-8 μg/dose or 0.5-15 μg/dose. Doses may be, for example, 0.5 μg/dose, 0.6 μg/dose, 0.7 μg/dose, 0.8 μg/dose, 0.9 μg/dose, 1.0 μg/dose, 2.0 μg/dose, 3.0 μg/dose, 3.5 μg/dose, 4.0 μg/dose, 4.5 μg/dose, 5.0 μg/dose, 5.5 μg/dose, 6.0 μg/dose, 6.5 μg/dose, 7.0 μg/dose, 7.5 μg/dose, 8.0 μg/dose, 9.0 μg/dose, 10.0 μg/dose, 11.0 μg/dose, 12.0 μg/dose, 13.0 μg/dose, 14.0 μg/dose, or 15.0 μg/dose. Doses may be adjusted depending upon the body mass, body area, weight, blood volume of the subject, or route of delivery. In one embodiment, 2 μg, 3 μg, 4 μg, 5 μg, 6 μg, 7 μg, 8 μg, 9 μg, 10 μg, 11 μg, or 12 μg of GLA in 1 ml is administered intratumorally. In this regard, the 1 mL dose of GLA may be injected in equal amounts in multiple zones of the tumor. In certain embodiments, about 0.01 mg/kg to about 100 mg/kg body weight of GLA will be administered, typically by the intradermal, intratumoral, subcutaneous, intramuscular or intravenous route, or by other routes. In certain embodiments, the dosage of GLA is about 0.1 mg/kg to about 1 mg/kg, and in certain embodiments, ranges from about 0.1 μg/kg, 0.2 μg/kg, 0.3 μg/kg, 0.4 μg/kg, 0.5 μg/kg, 0.6 μg/kg, 0.7 μg/kg, 0.8 μg/kg, 0.9 μg/kg, 1 μg/kg, 2 μg/kg, 3 μg/kg, 4 μg/kg, 5 mg/kg, 6 mg/kg, 7 mg/kg, 8 mg/kg, 9 mg/kg, 10 mg/kg to about 200 mg/kg. It will be evident to those skilled in the art that the number and frequency of administration will be dependent upon the response of the host. As described herein, the appropriate dose may also depend upon the patient's (e.g., human) condition, that is, stage of the disease, general health status, as well as age, gender, and weight, and other factors familiar to a person skilled in the medical art. As noted elsewhere herein, the GLA compositions described herein do not include antigen.

### Delivery of the Lentiviral Particles

The viral particles for delivery of the engineered TCRs described herein (e.g. retroviral, lentiviral or other viral particles) may be delivered to a target cell in any way that allows the virus to contact the target cell, e.g., T cell, NK cell or dendritic cell, in which delivery of a polynucleotide of interest is desired. At times, a suitable amount of virus will be introduced into a human or other animal directly (in vivo), e.g., though injection into the body. Suitable animals include, without limitation, horses, dogs, cats, cattle, pigs, sheep, rabbits, chickens or other birds. Viral particles may be injected by a number of routes, such as intravenous, intra-dermal, subcutaneous, intranodal, intra-peritoneal cavity, or mucosal. The virus may be delivered using a subdermal injection device such the devices disclosed in U.S. Pat. Nos. 7,241,275, 7,115,108, 7,108,679, 7,083,599, 7,083,592, 7,047,070, 6,971,999, 6,808,506, 6,780,171, 6,776,776, 6,689,118, 6,670,349, 6,569,143, 6,494,865, 5,997,501, 5,848,991, 5,328,483, 5,279,552, 4,886,499, all of which are incorporated by reference in their entirety. Other injection locations also are suitable, such as directly into organs comprising target cells. For example, intra-lymph node injection, intra-spleen injection, or intra-bone marrow injection may be used to deliver virus to the lymph node, the spleen and the bone marrow, respectively. Depending on the particular circumstances and nature of the target cells, introduction can be carried out through other means including for example, inhalation, or direct contact with epithelial tissues, for example those in the eye, mouth or skin.

Alternatively, target cells are provided and contacted with the virus in vitro, such as in culture plates. The target cells are typically populations of cells comprising dendritic cells or T cells obtained from a healthy subject or a subject in need of treatment or in whom it is desired to stimulate an immune response to an antigen. Methods to obtain cells from a subject are well known in the art and includes phlebotomy, surgical excision, and biopsy. Human DCs may also be generated by obtaining CD34α+ human hematopoietic progenitors and using an in vitro culture method as described elsewhere (e.g., Banchereau et al. Cell 106, 271-274 (2001) incorporated by reference in its entirety).

The virus may be suspended in media and added to the wells of a culture plate, tube or other container. Media containing the virus may be added prior to the plating of the cells or after the cells have been plated. Cells are typically incubated in an appropriate amount of media to provide viability and to allow for suitable concentrations of virus in the media such that transduction of the host cell occurs. The cells are preferably incubated with the virus for a sufficient amount of time to allow the virus to infect the cells. Preferably the cells are incubated with virus for at least 1 hour, at least 5 hours or at least 10 hours.

In both in vivo and in vitro delivery, an aliquot of viral particles containing sufficient number to infect the desired target cells may be used. When the target cell is to be cultured, the concentration of the viral particles is generally at least 1 IU/μL, more preferably at least 10 IU/μ1, even more preferably at least 300 IU/μL, even more preferably at least 1×10⁴ IU/μL, even more preferably at least 1×10⁵ IU/μL, even more preferably at least 1×10⁶IU/μL, or even more preferably at least 1×10⁷ IU/μL.

Following infection with the virus in vitro, target cells can be introduced (or re-introduced) into a human or other animal. The cells can be introduced into the dermis, under the dermis, or into the peripheral blood stream. The cells introduced into an animal are preferably cells derived from that animal, to avoid an adverse immune response. Cells derived from a donor having a similar immune background may also be used. Other cells that also can be used include those designed to avoid an adverse immunologic response.

Target cells may be analyzed for integration, transcription and/or expression of the sequence or gene(s) of interest, the number of copies of the gene integrated, and the location of the integration, for example. Such analysis may be carried out at any time and may be carried out by any method known in the art.

Subjects in which a virus, or virus-infected T cells, are administered can be analyzed for location of infected cells, expression of the virus-delivered polynucleotide or gene of interest, stimulation of an immune response, and monitored for symptoms associated with a disease or disorder by any methods known in the art.

The methods of infecting cells disclosed herein do not depend upon individual-specific characteristics of the cells. As a result, they are readily extended to a variety of animal species. In some instances, viral particles are delivered to a human or to human T cells, and in other instances they are delivered to an animal such as a mouse, horse, dog, cat, or mouse or to birds. As discussed herein, the viral vector genome is pseudotyped to confer upon it a broad host range as well as target cell specificity. One of skill in the art would also be aware of appropriate internal promoters and other elements to achieve the desired expression of a sequence of interest in a particular animal species. Thus, one of skill in the art will be able to modify the method of infecting dendritic cells from any species.

### Combination Therapy

The therapeutic compositions described herein may also be administered simultaneously with, prior to, or after administration of one or more other therapeutic agents. Such combination therapy may include administration of a single pharmaceutical dosage formulation which contains a therapeutic composition described herein and one or more additional active agents, as well as administration of compositions (e.g., compositions comprising an engineered TCR as described herein, or compositions comprising lentiviral vector particles comprising a sequence encoding an engineered TCR as described herein or compositions comprising isolated T cells modified to express an engineered TCR as described herein) and each active agent in its own separate pharmaceutical dosage formulation. For example, a therapeutic composition as described herein and the other active agent can be administered to the mammalian subject together in a single oral dosage composition such as a tablet or capsule, or each agent administered in separate oral dosage formulations. Similarly, the compositions described herein (e.g., comprising the lentiviral vector particles comprising a sequence encoding an engineered TCR as described herein, or a composition comprising T cells modified ex vivo with the such particles, or compositions comprising an engineered TCR) and the other active agent can be administered to the mammalian subject together in a single parenteral dosage composition such as in a saline solution or other physiologically acceptable solution, or each agent administered in separate parenteral dosage formulations. Where separate dosage formulations are used, the compositions disclosed herein and one or more additional active agents can be administered at essentially the same time, i.e., concurrently, or at separately staggered times, i.e., sequentially and in any order; combination therapy is understood to include all these regimens. Thus, in certain embodiments, also contemplated is the administration of one or more compositions disclosed herein, in combination with one or more other therapeutic agents. Such therapeutic agents may be accepted in the art as a standard treatment for cancer. Exemplary therapeutic agents contemplated include cytokines, growth factors, immune checkpoint inhibitors, TLR agonists including TLR4 agonists such as glucopyranosyl lipid adjuvant (GLA) (as described for example in U.S. Pat. No. 8,273,361, WO2008/153541 and WO2009143457, the disclosure of which are incorporated herein by reference in their entireties), steroids, NSAIDs, DMARDs, anti-inflammatories, chemotherapeutics, radiotherapeutics, or other active and ancillary agents.

In certain embodiments, the therapeutic compositions disclosed herein may be administered in conjunction with any number of immune checkpoint inhibitors. Immune checkpoint inhibitors include antibodies, or antigen binding fragments thereof, that bind to and block or inhibit the activity of one or more of CTLA-4, PDL1, PDL2, PD1, B7-H3, B7-H4, BTLA, HVEM, TIM3, and GALS. Illustrative immune checkpoint inhibitors include Tremelimumab (CTLA-4 blocking antibody), anti-OX40, PD-L1 monoclonal Antibody (Anti-B7-H1; MEDI4736), ipilimumab, MK-3475 (PD-1 blocker). Nivolumamb (anti-PD1 antibody).

In certain embodiments, the compositions disclosed herein may be administered in conjunction with any number of chemotherapeutic agents. Examples of chemotherapeutic agents include alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pento statin; phenamet; pirarubicin; podophyllinic acid; 2-ethylhydrazide; procarbazine; PSK®; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOL®, Bristol-Myers Squibb Oncology, Princeton, N.J.) and doxetaxel (TAXOTERE®, Rhne-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid derivatives such as Targretin™ (bexarotene), Panretin™ (alitretinoin); ONTAK™ (denileukin diftitox); esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the present disclosure provides a method of treating, inhibiting the progression of or preventing a cancer associated with NY-ESO-1 expression by administering to a mammalian subject afflicted by a cancer associated with NY-ESO-1 expression a therapeutically effective amount of an engineered TCR disclosed herein, lentiviral vectors comprising a nucleic acid encoding an engineered TCR disclosed herein, or a composition comprising T cells modified ex vivo with such particles, and then further administering to the patient a composition comprising a pseudotyped lentiviral vector particle comprising an envelope that targets dendritic cells and can thus be used for dendritic cell vaccination (see e.g., U.S. Pat. Nos. 8,329,162; 8,372,390; 8,273,345; 8,187,872; 8,323,662 and published PCT application WO2013/149167). In this manner, antigen specific T-cells can be generated through in vivo or ex vivo genetic modification using the lentiviral vectors described herein, and then are boosted in vivo through active immunization of dendritic cells, using a DC-tropic lentiviral vector.

In another embodiment, the present disclosure provides a method of treating, inhibiting the progression of or preventing an NY-ESO-1 cancer by administering to a mammalian subject afflicted with the NY-ESO-1 cancer a therapeutically effective amount of a composition comprising an engineered TCR disclosed herein, a composition comprising lentiviral vectors comprising a nucleic acid encoding an engineered TCR disclosed herein, or a composition comprising T cells modified ex vivo with such particles, and then further boosting the immune response by administering to the patient a composition comprising a TLR4 agonist, such as Glucopyranosyl Lipid A (GLA) (see e.g., U.S. Pat. No. 8,273,361 and published applications WO2012/141984 and US20120328655), with or without an antigen. In this manner, antigen specific T-cells can be generated through in vivo or ex vivo genetic modification using the lentiviral vectors described herein, and then are boosted in vivo through activation of dendritic cells.

For purposes of the inventive methods, wherein host cells or populations of cells are administered to the subject, the cells can be cells that are allogeneic or autologous to the host. Preferably, the cells are autologous to the subject.

The subject referred to herein can be any subject. Preferably, the subject is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

### Pharmaceutical Compositions and Kits

Also contemplated herein are pharmaceutical compositions and kits containing one or more of (1) an engineered TCR as described herein; (2) viral particles comprising a nucleic acid encoding an engineered TCR; (3) immune cells, such as T cells or NK cells, modified to express an engineered TCR as described herein; (4) nucleic acids encoding an engineered TCR as described herein. In some embodiments, the present disclosure provides compositions comprising lentiviral vector particles comprising a nucleotide sequence encoding an engineered TCR described herein (or T cells that have been modified using the vector particles described herein to express an engineered TCR). Such compositions can be administered to subjects in the methods of the present disclosure as described further herein.

Compositions comprising the modified T cells as described herein can be utilized in methods and compositions for adoptive immunotherapy in accordance with known techniques, or variations thereof that will be apparent to those skilled in the art based on the instant disclosure. See, e.g., US Patent Application Publication No. 2003/0170238 to Gruenberg et al; see also U.S. Pat. No. 4,690,915 to Rosenberg, the disclosure of which are incorporated herein by reference in their entireties.

In some embodiments, the cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin.

A treatment-effective amount of cells in the composition is typically greater than 10² cells, and up to 10⁶, up to and including 10⁸ or 10⁹ cells and can be more than 10¹⁰ cells. The number of cells will depend upon the ultimate use for which the composition is intended as will the type of cells included therein. For example, if cells that are specific for a particular antigen are desired, then the population will contain greater than 70%, generally greater than 80%, 85% and 90-95% of such cells. For uses provided herein, the cells are generally in a volume of a liter or less, can be 500 ml or less, even 250 ml or 100 ml or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁹, 10¹⁰ or 10¹¹ cells.

Pharmaceutical compositions provided herein can be in various forms, e.g., in solid, liquid, powder, aqueous, or lyophilized form. Examples of suitable pharmaceutical carriers are known in the art. Such carriers and/or additives can be formulated by conventional methods and can be administered to the subject at a suitable dose. Stabilizing agents such as lipids, nuclease inhibitors, polymers, and chelating agents can preserve the compositions from degradation within the body. In a composition intended to be administered by injection, one or more of a surfactant, preservative, wetting agent, dispersing agent, suspending agent, buffer, stabilizer and isotonic agent may be included.

The engineered TCRs as described herein, or the viral vector particles comprising a nucleotide sequence encoding an engineered TCR provided herein, can be packaged as kits. Kits can optionally include one or more components such as instructions for use, devices, and additional reagents, and components, such as tubes, containers and syringes for practice of the methods. Exemplary kits can include the nucleic acids encoding the engineered TCRs, the engineered TCR polypeptides, or viruses provided herein, and can optionally include instructions for use, a device for detecting a virus in a subject, a device for administering the compositions to a subject, and a device for administering the compositions to a subject.

Kits comprising polynucleotides encoding a gene of interest (e.g., an engineered TCR) are also contemplated herein. Kits comprising a viral vector encoding a sequence of interest (e.g., an engineered TCR) and optionally, a polynucleotide sequence encoding an immune checkpoint inhibitor are also contemplated herein.

Kits contemplated herein also include kits for carrying out the methods for detecting the presence of polynucleotides encoding any one or more of the public TCR VβCDR3 sequences disclosed herein. In particular, such diagnostic kits may include sets of appropriate amplification and detection primers and other associated reagents for performing deep sequencing to detect the polynucleotides encoding the public TCR VβCDR3 sequences disclosed herein. In further embodiments, the kits herein may comprise reagents for detecting the TCR polypeptide comprising the TCR VβCDR3, such as antibodies or other binding molecules. Diagnostic kits may also contain instructions for determining the presence of the polynucleotides encoding the public TCR VβCDR3 sequences or for determining the presence of the TCR polypeptides comprising the public TCR VβCDR3s disclosed herein.

A kit may also contain instructions. Instructions typically include a tangible expression describing the virus and, optionally, other components included in the kit, and methods for administration, including methods for determining the proper state of the subject, the proper dosage amount, and the proper administration method, for administering the virus. Instructions can also include guidance for monitoring the subject over the duration of the treatment time.

Kits provided herein also can include a device for administering a composition described herein to a subject. Any of a variety of devices known in the art for administering medications or vaccines can be included in the kits provided herein. Exemplary devices include, but are not limited to, a hypodermic needle, an intravenous needle, a catheter, a needle-less injection device, an inhaler, and a liquid dispenser, such as an eyedropper. Typically, the device for administering a virus of the kit will be compatible with the virus of the kit; for example, a needle-less injection device such as a high pressure injection device can be included in kits with viruses not damaged by high pressure injection, but is typically not included in kits with viruses damaged by high pressure injection.

Kits provided herein also can include a device for administering a compound, such as a T cell activator or stimulator, or a TLR agonist, such as a TLR4 agonist (see e.g., U.S. Pat. No. 8,273,361, the disclosure of which is incorporated herein by reference in its entirety), to a subject. Any of a variety of devices known in the art for administering medications to a subject can be included in the kits provided herein. Exemplary devices include a hypodermic needle, an intravenous needle, a catheter, a needle-less injection, but are not limited to, a hypodermic needle, an intravenous needle, a catheter, a needle-less injection device, an inhaler, and a liquid dispenser such as an eyedropper. Typically, the device for administering the compound of the kit will be compatible with the desired method of administration of the compound.

The following examples are offered by way of illustration, and not by way of limitation.

### EXAMPLES

### Example 1: Treatment with LV305 Increased the Polyclonal Affinity of NY-ESO-1 Specific T Cells Post-Therapy

This Example demonstrates that treatment with LV305 increased the polyclonal affinity T cells that recognize NY-ESO-1 tumor antigen as measured by ELISPOT.

In this study on LV305, the patient received 3 injections with LV305, a dendritic cell tropic lentivector encoding the NY-ESO-1 tumor antigen. T cell response to NY-ESO-1 in pre-vaccination (pre-Tx) and post-vaccination (post-Tx) PBMC samples was measured by ELISPOT, in which the cells were stimulated with NY-ESO-1 peptide mix for 40 hr and the number of T cells that secreted IFN-γ was measured by counting the spots in plates that were pre-coated with anti-IFN-7 antibody.

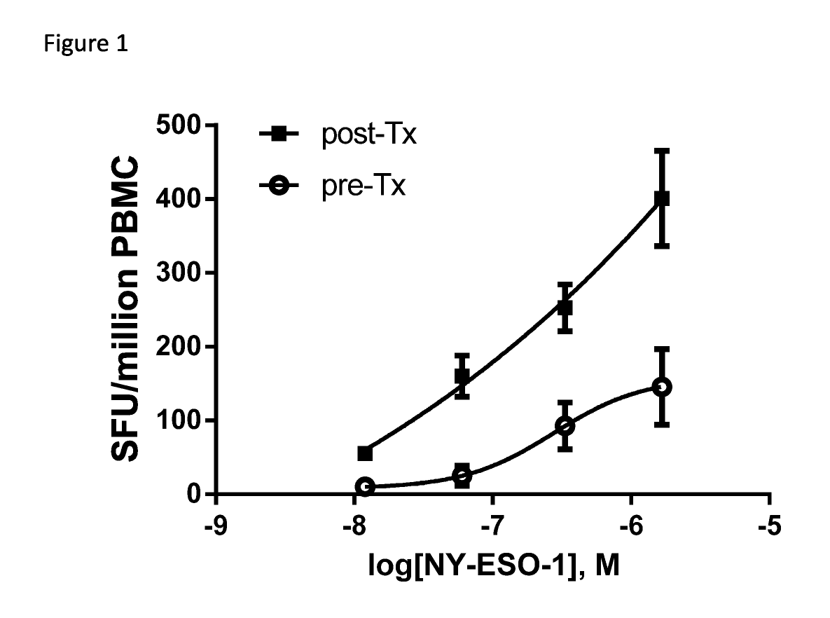

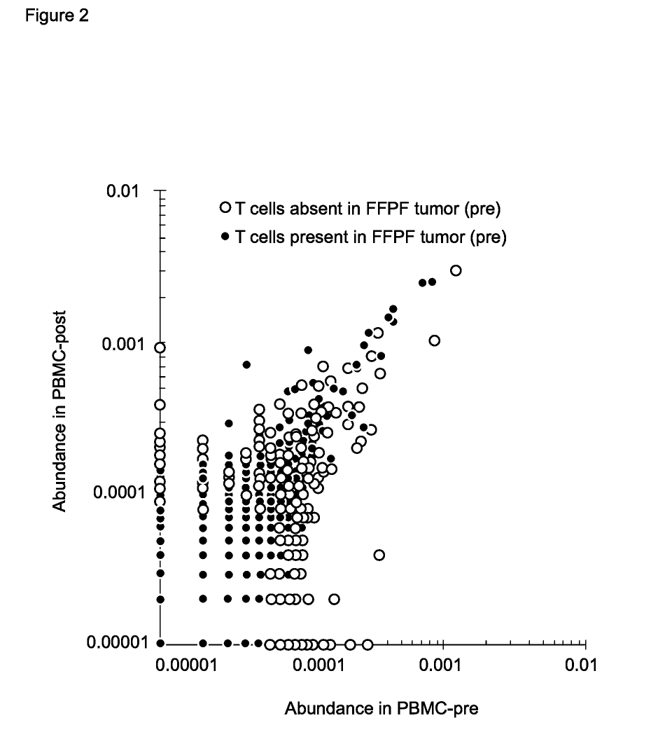

As shown in FIG. 1, post-Tx T cells had higher response to NY-ESO-1 peptide mix than pre-Tx T cells, at all the NY-ESO-1 concentrations tested (1670, 334, 60, and 12 nM). At the two lower concentrations we tested (12 and 60 nM), there was no detectable T cell response in pre-Tx samples, yet there was significant T cell response in post-Tx sample. These data demonstrate that treatment with LV305 enhances T cell response to NY-ESO-1 and resulted in higher affinity NY-ESO-1 specific T cells, which recognize the antigen at low concentrations of NY-ESO-1 peptides.

### Example 2: Tumor Antigen-Specific TCR Sequences are Enriched in Post-Tx PBMC as Compared to Pre-Tx PBMC

This Example demonstrates that treatment with LV305 resulted enrichment of NY-ESO-1 specific TCR sequences in the peripheral blood.

In this study, PBMC were collected from the patient before LV305 treatment and after three vaccinations with LV305. A pre-Tx tumor sample was also collected from the patient. The PBMC and tumor sample were subjected to DNA extraction and subsequent sequencing analysis of the T cell receptor (TCR) beta chain. The sequence similarity between pre-Tx and post-Tx PBMC was analyzed using scatter plot. Then the TCR sequence from the tumor sample was also compared to the pre-Tx and post-Tx PBMC for similarity. The result showed that the TCR sequence from the T cells infiltrating the tumor samples are enriched in post-Tx PBMC as compared to pre-Tx PBMC.

### Example 3: An Oligoclonal Culture that is Highly Enriched for NY-ESO-1 Specific T Cells has been Established from Post-Tx PBMC

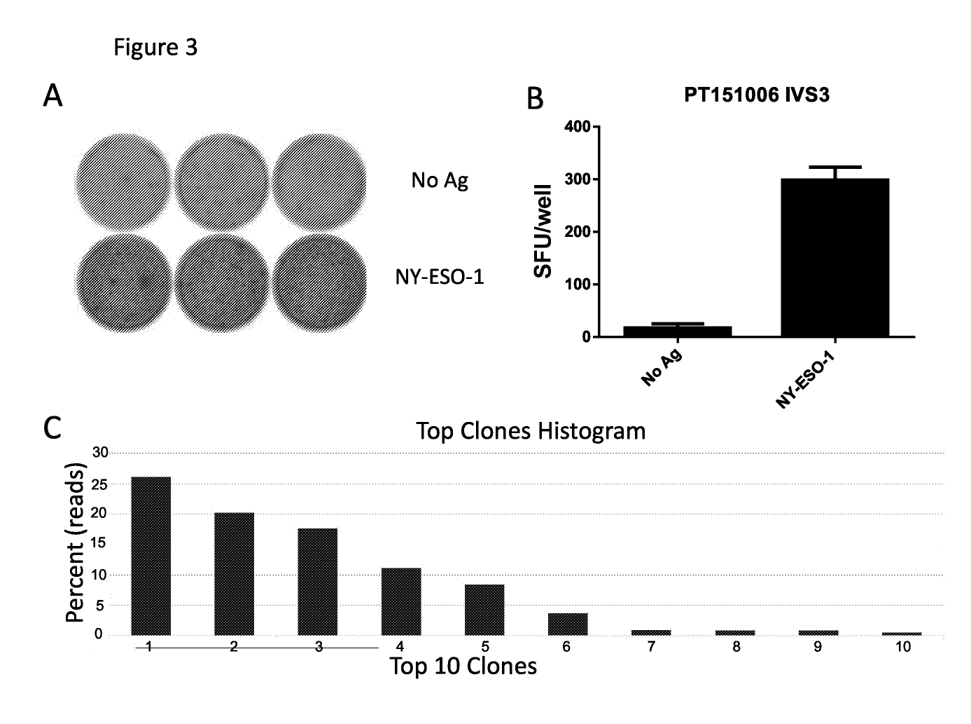

This Example demonstrates that an oligoclonal culture that is highly enriched for NY-ESO-1 specific T cells has been established from post-Tx PBMC. The culture was started by using PBMC from a patient after vaccination with LV305. The PBMC was cultured in OpTmizer T cell expansion medium (Invitrogen, Carlsbad, Calif.) with NY-ESO-1 overlapping peptide (0.5 ug/mL, JPT Technologies, Berlin, Germany) in the presence of IL-2 and IL-7 (10 ng/mL). After repeated stimulation and long-term culture (>3 months), the PBMC culture was highly enriched for NY-ESO-1 specific T cells. As shown in FIG. 3, the enriched T cells secreted high amount of IFN-γ upon stimulation with NY-ESO-1 peptides. TCR sequencing analysis showed that the culture is very oligoclonal as the top 6 clones accounts for more than 90% of all the T cells.

### Example 4: TCRβ CDR3 Sequences in the Oligoclonal Culture are Enriched in Post-Tx PBMC as Compared to Pre-Tx PBMC

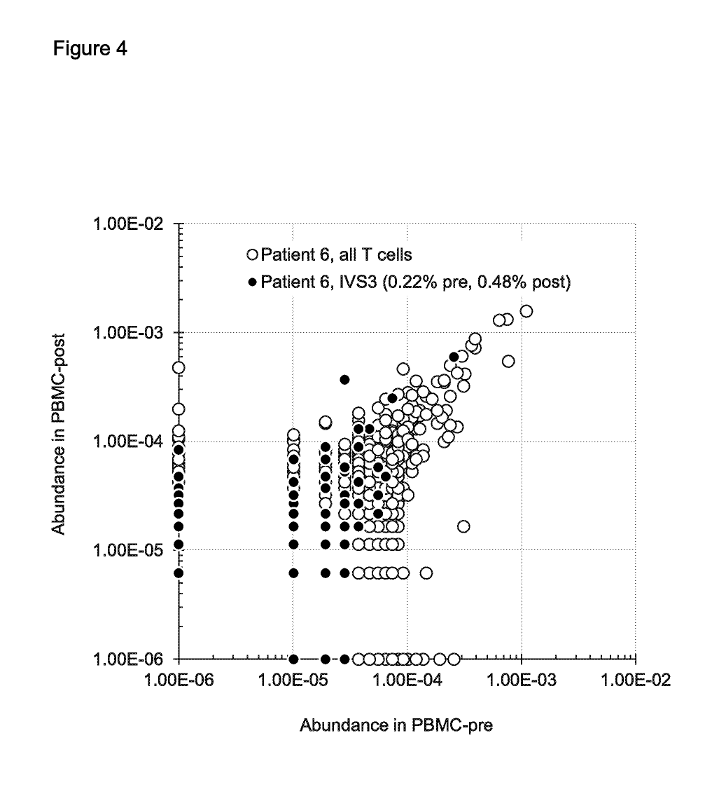

This Example demonstrates that the TCR sequences from the NY-ESO-1 stimulated oligoclonal culture (PT151006 IVS3) are enriched in post-Tx PBMC as compared to pre-Tx PBMC. The sequence similarity between pre-Tx and post-Tx PBMC was analyzed using scatter plot (FIG. 4). Then the top 6 TCR sequences from PT151006 IVS3 were also compared to the pre-Tx and post-Tx PBMC for similarity. The result showed that the TCR sequences from PT151006 IVS3, are enriched in post-Tx PBMC as compared to pre-Tx PBMC.

### Example 5: A TCRβ CDR3 Clone with a Frequency of 20.5% in Pt151006 IVS3 can be Detected in Pt151016 Post-Tx PBMC

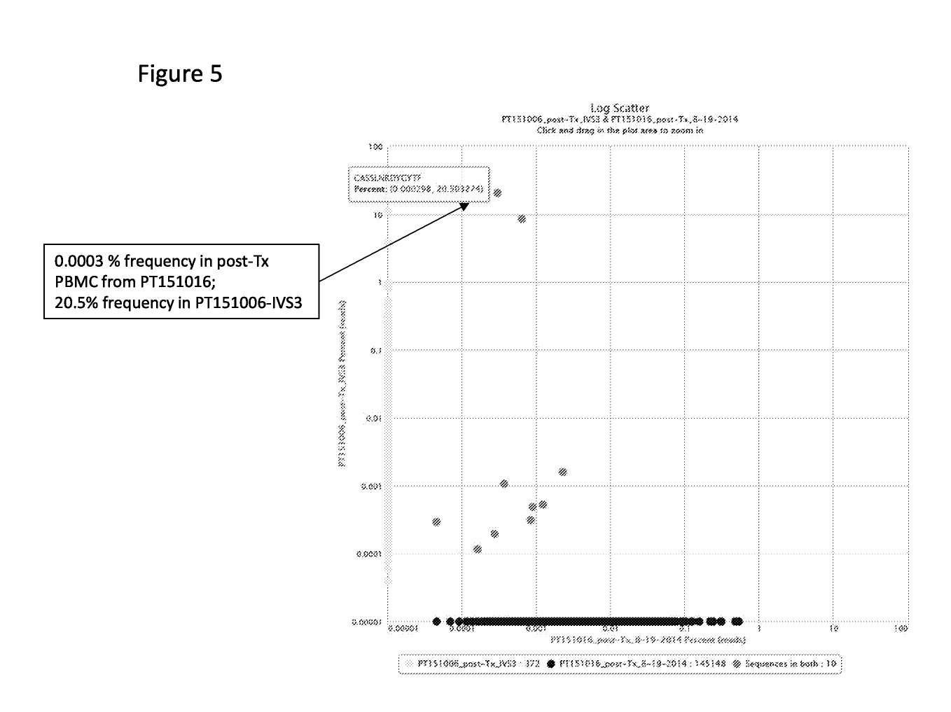

This Example demonstrates that the second dominant clone from PT151006 IVS3 is also detected in PT151016 post-Tx PBMC (FIG. 5). Of note, the two patients have different Class I HLA background (see Table 4). The CDR3 sequence of the TCRβ chain of the 2^{nd} dominant clone (20.5% frequency) from IVS3 is CASSLNRDYGYTF (SEQ ID NO: 2). As shown in FIG. 5, this amino sequence is detected at 0.0003% frequency in the post-Tx PBMC from PT151016, while it is not detected in the pre-Tx PBMC from PT151016.

### Example 6: A TCRB CDR3 Clone with a Frequency of 8.5% in Pt151006 IVS3 can be Detected in Pt151016 Post-Tx PBMC

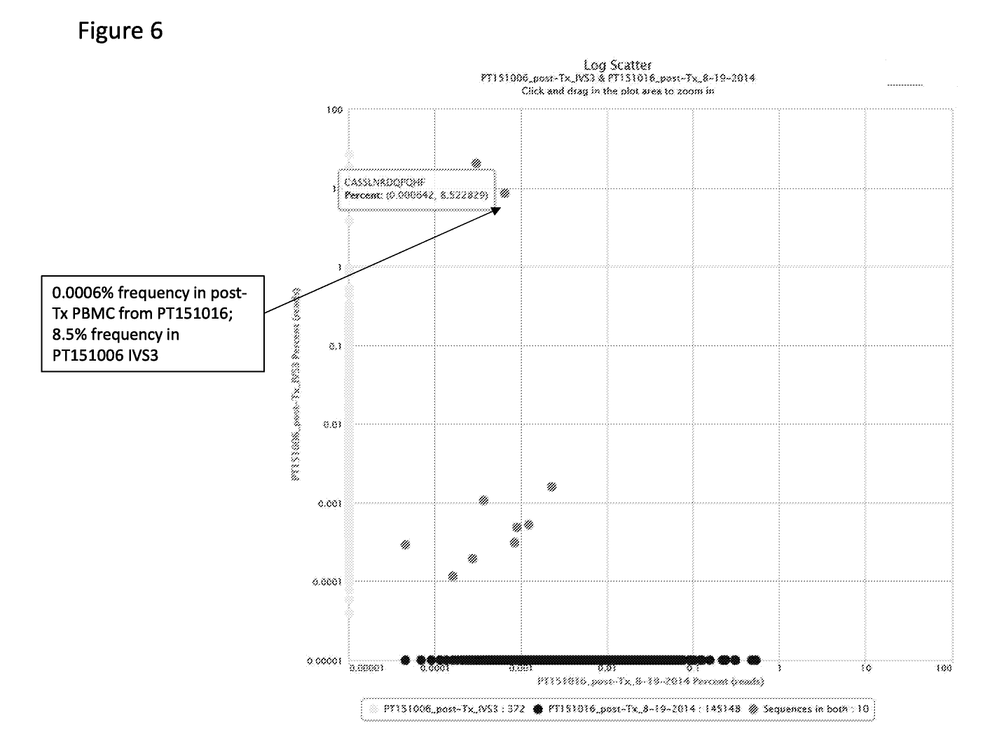

This Example demonstrates that the fifth dominant clone from PT151006 IVS3 is also detected in PT151016 post-Tx PBMC. The CDR3 sequence of the TCRbeta chain of the 5^{th} dominant clone (frequency 8.5%) from IVS3 is CASSLNRDQPQHF (SEQ ID NO: 3). As shown in FIG. 6, this amino sequence is detected at 0.0006% frequency in the post-Tx PBMC from PT151016, while it is not detected in the pre-Tx PBMC from PT151016.

### Example 7: A TCRB CDR3 Clone with a Frequency of 26.2% in Pt151006 IVS3 can be Detected in Pt151014 Post-Tx PBMC

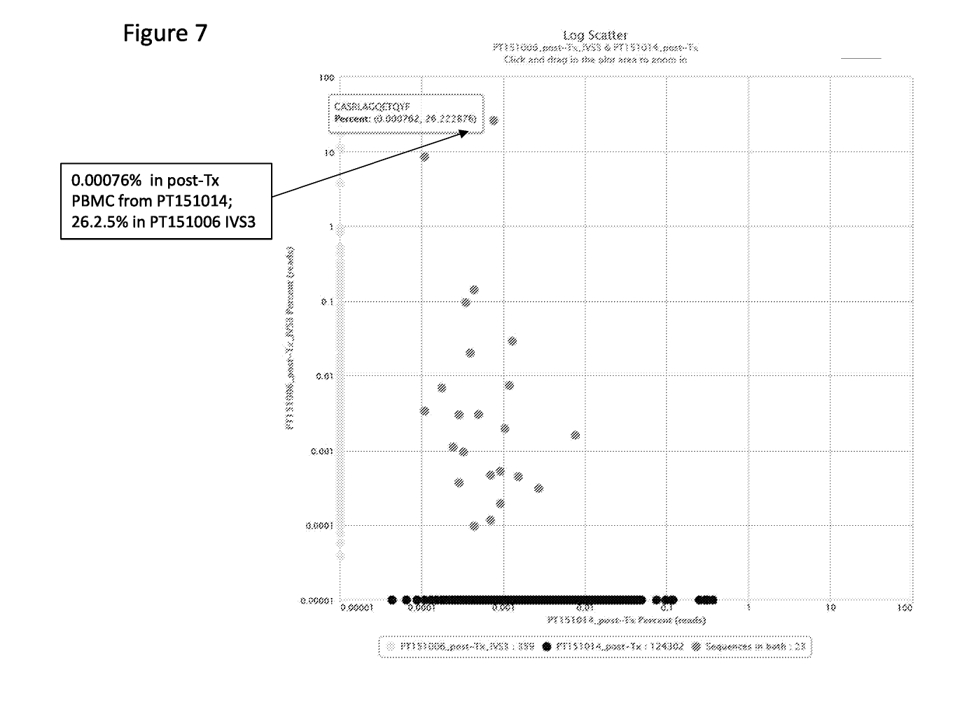

This Example demonstrates that the dominant clone from PT151006 IVS3 (26.2% frequency) is also detected in PT151014 post-Tx PBMC. The CDR3 sequence of this clone is CASRLAGQETQYF (SEQ ID NO: 4). As shown in FIG. 7, this amino sequence is detected at 0.000762% frequency in the post-Tx PBMC from PT151014, while it is not detected in the pre-Tx PBMC from PT151014.

### Example 8: Three Out of the Top Six TCRβ CDR3 Clones are Public Clones that are Shared Between More than One Patient

This Example demonstrates that the three public sequences we discovered are detected in multiple patients with different HLA background and increase in frequency in PBMC sampled post LV305 therapy.

The first public CDR3 sequence of TCR is CASSLNRDYGYTF (SEQ ID NO: 2). As shown in Table 1, this sequence is detected at 0.001% in pre-Tx PBMC from PT151006, and increased to 0.003% in post-Tx PBMC from the same patient. This sequence is non-detectable (0%) in pre-Tx PBMC from PT151016 and can be detected at 0.0003% in post-Tx PBMC from the same patient. This sequence is non-detectable (0%) in pre-Tx PBMC from PT151050 and can be detected at 0.0003% in post-Tx PBMC from the same patient.

TABLE 1
Frequency of the 1st Public TCRβ CDR3
Sequence, CASSLNRDYGYTF, in eight patients

| PT: | 151006 | 151014 | 151016 | 151035 | 151039 | 151050 | 151119 | 151070 |
|---|---|---|---|---|---|---|---|---|
| Pre-Tx | 0.001% | 0% | 0% | 0% | 0% | 0% | 0% | 0% |
| PBMC | | | | | | | | |
| Post-Tx | 0.003% | 0% | 0.0003% | 0% | 0% | 0.0003% | 0% | 0% |
| PBMC | | | | | | | | |
| IVS3 | 20.5% | | | | | | | |
| from | | | | | | | | |
| PT151006 | | | | | | | | |

Table 1. The frequency of a 1st public TCRβ CDR3 sequence in pre-Tx and post-Tx PBMC samples from eight patients. This table shows the frequency of the CDR3 sequence, CASSLNRDYGYTF (SEQ ID NO: 2), in different patient PBMC samples collected either before or after treatment with LV305. For example, this sequence is detected at 0.001% in pre-Tx PBMC from PT151006, and increased to 0.003% in post-Tx PBMC from the same patient. This sequence is non-detectable (0%) in pre-Tx PBMC from PT151016 and can be detected at 0.0003% in post-Tx PBMC from the same patient. This sequence is non-detectable (0%) in pre-Tx PBMC from PT151050 and can be detected at 0.0003% in post-Tx PBMC from the same patient.

The second public CDR3 sequence of TCR is CASSLNRDQPQHF (SEQ ID NO: 3). As shown in Table 2, this sequence is detected at 0.0058% in pre-Tx PBMC from PT151006, and increased to 0.017% in post-Tx PBMC from the same patient. The sequence can also be detected in a pre-Tx tumor biopsy from this patient (0.06%) and a tumor infiltrating lymphocytes (TIL) culture from the same patient, 0.002% in TIL-PC12-04A1. This sequence is non-detectable (0%) in pre-Tx PBMC from PT151014 and can be detected at 0.000109% in post-Tx PBMC from the same patient. This sequence is non-detectable (0%) in pre-Tx PBMC from PT151050 and can be detected at 0.0012% in post-Tx PBMC from the same patient. Overall, this TCR is detectable in 6 out of 8 patients, increased in frequency post-Tx sample in 5/8 patients and decreased in 1/8 patient.

TABLE 2
Frequency of the 2nd Public TCRβ CDR3 Sequence, CASSLNRDQPQHF, in eight patients

| PT: | 151006 | 151014 | 151016 | 151035 | 151039 | 151050 | 151119 | 151070 |
|---|---|---|---|---|---|---|---|---|
| Pre-Tx | 0.0058% | 0% | 0% | 0.001% | 0% | 0% | 0% | 0% |
| PBMC | | | | | | | | |
| Post-Tx | 0.017% | 0.000109% | 0.0012% | 0% | 0% | 0.0006% | 0.000893% | 0% |
| PBMC | | | | | | | | |
| IVS3 | 8.5% | | | | | | | |
| from | | | | | | | | |
| PT151006 | | | | | | | | |
| TIL from | 0.002% | | | | | | | |
| PT151006 | | | | | | | | |
| Fixed | 0.06% | | | | | | | |
| tumor | | | | | | | | |
| from | | | | | | | | |
| PT151006 | | | | | | | | |

Table 2. The frequency of a 2nd public TCRβ CDR3 sequence in pre-Tx and post-Tx PBMC samples from eight patients. This table shows the frequency of the CDR3 sequence, CASSLNRDQPQHF (SEQ ID NO: 3), in different patient PBMC samples collected either before or after treatment with LV305. For example, this sequence is detected at 0.0058% in pre-Tx PBMC from PT151006, and increased to 0.017% in post-Tx PBMC from the same patient. The sequence can also be detected from fixed tumor from this patient (0.06%) and a TIL culture from this patient, 0.000647%. This sequence is non-detectable (0%) in pre-Tx PBMC from PT151016 and can be detected at 0.0006% in post-Tx PBMC from the same patient. This sequence is non-detectable (0%) in pre-Tx PBMC from PT151050 and can be detected at 0.0012% in post-Tx PBMC from the same patient.

The third public CDR3 sequence of TCR is CASRLAGQETQYF (SEQ ID NO: 4). As shown in Table 3, this sequence is 0% in pre-Tx PBMC from PT151006, and increased to 0.000196% in post-Tx PBMC from the same patient. This sequence is non-detectable (0%) in pre-Tx PBMC from PT151-014 and can be detected at 0.000761% in post-Tx PBMC from the same patient. This sequence is also detected at 0.000274% in pre-Tx PBMC from PT151050.

TABLE 3
Frequency of the 3^{rd} Public TCRβ CDR3 Sequence, CASRLAGQETQYF, in eight patients

| PT: | 151006 | 151014 | 151016 | 151035 | 151039 | 151050 | 151119 | 151070 |
|---|---|---|---|---|---|---|---|---|
| Pre-Tx | 0% | 0% | 0% | 0% | 0% | 0.000274% | 0% | 0% |
| PBMC | | | | | | | | |
| Post-Tx | 0.000196% | 0.00076% | 0% | 0% | 0% | 0% | 0% | 0% |
| PBMC | | | | | | | | |
| IVS3 | 26% | | | | | | | |
| PT151006 | | | | | | | | |

Table 3. The frequency of a 3rd public TCRβ CDR3 sequence in pre-Tx and post-Tx PBMC samples from eight patients. This table shows the frequency of the CDR3 sequence, CASRLAGQETQYF (SEQ ID NO: 4), in different patient PBMC samples collected either before or after treatment with LV305. The sequence can be detected in PT151014 and PT151050 in addition to PT151006.

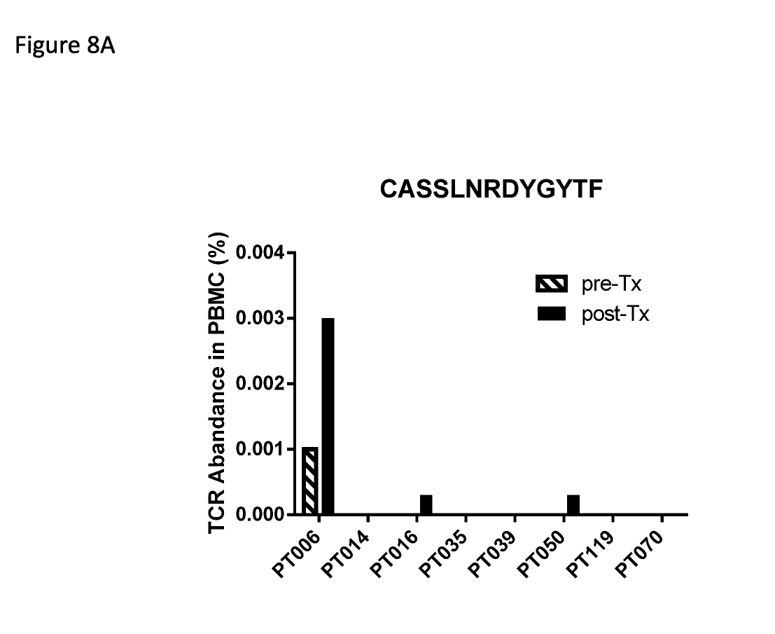

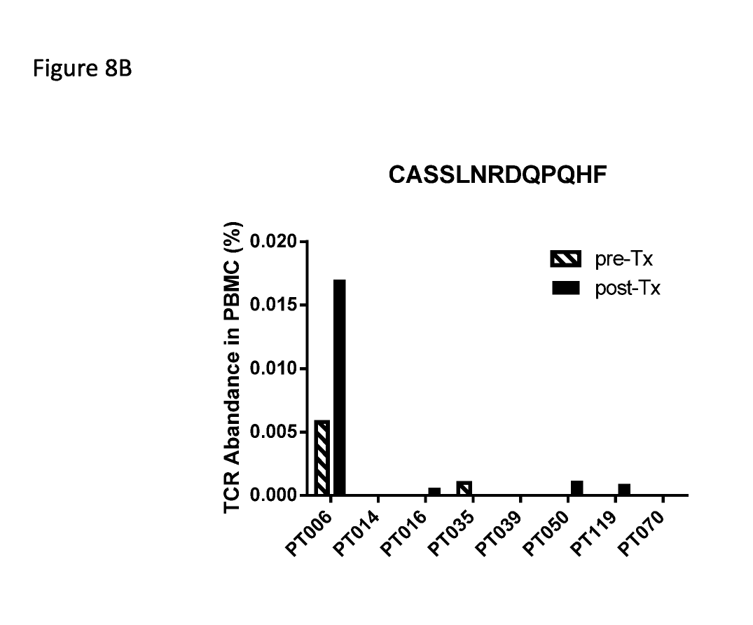

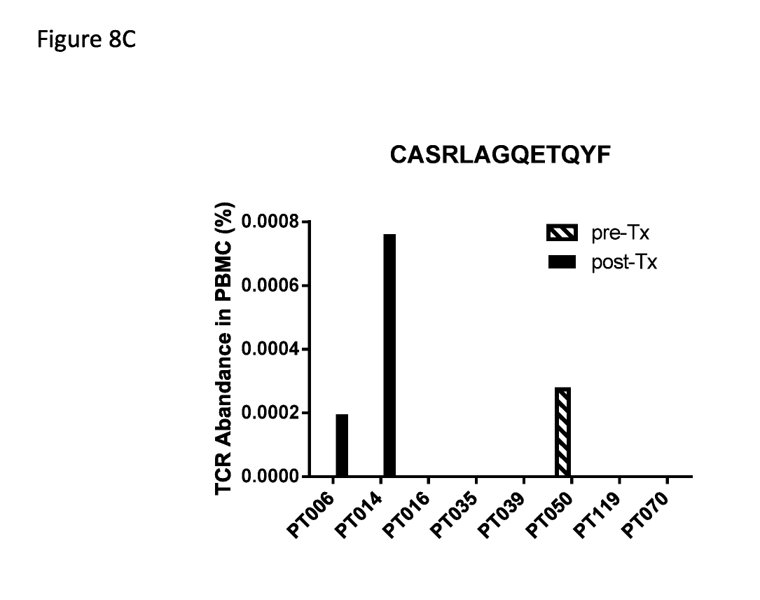

The frequency of the three identified public TCRs in pre-Tx and post-Tx PBMC from eight patients is summarized in FIG. 8.

### Example 9: The CDR3 of the Three Identified Public TCRB has the Typical Features of a Public TCR

This Example demonstrates that the identified TCRβ CDR3 sequences have the typical features of public TCRs: (1) they are encoded by different nucleotide sequences in different patients; (2) they have relatively short CDR3 lengths; and (3) they have a relatively limited untemplated nucleotide addition. All of these features are characteristic of public TCR sequences (Venturi Nat Rev Immunol 2008).

Diversification of the TCR Vα and Vβ gene segments depends on the use of different CDR1 and CDR2 regions (encoded in different V gene segments). CDR3 is created by the juxtaposition of different V(D)J germline segments after somatic recombination, with the diversity of the naive TCR repertoire increased further by a lack of precision during V(D)J gene rearrangement and by the addition of non-template encoded nucleotides (N) at the V(D)J junctions (Turner, et al, Nature Review Immunology 2006).

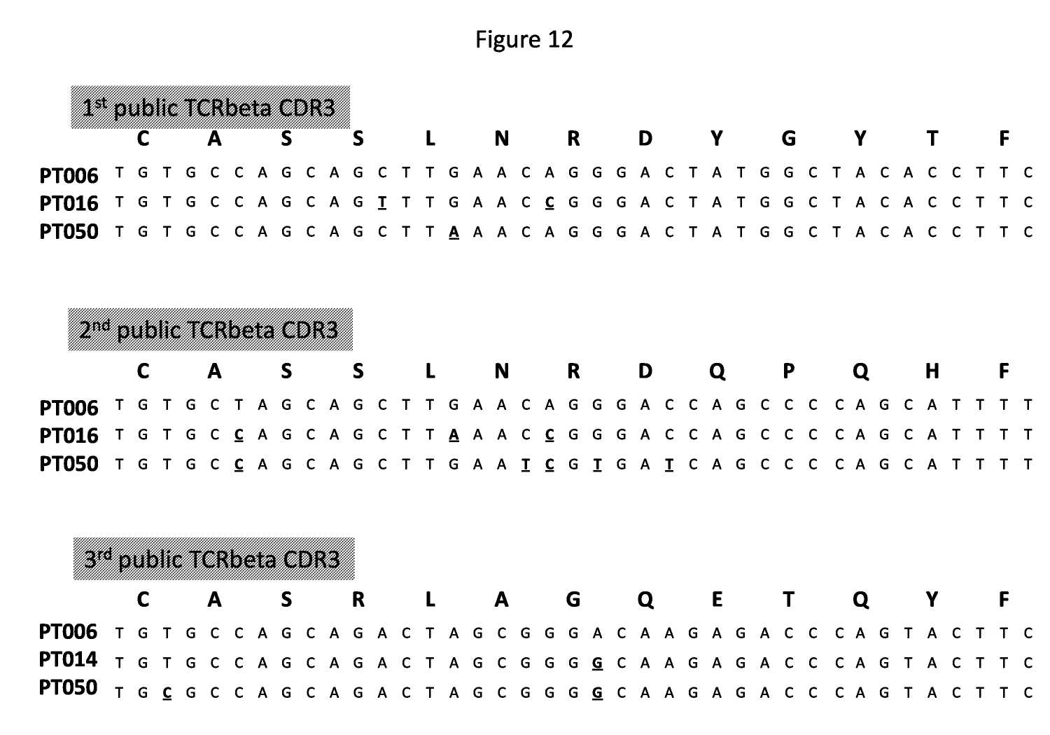

One possible way that public TCRs are generated is through convergent recombination. That is, many different V(D)J recombination events “converge” to produce the same nucleotide sequence and many different nucleotide sequences “converge” to encode the same amino-acid sequence (Venturi, et al, Nature Review Immunology 2008). As shown in FIG. 9 and FIG. 12, the public TCR sequences we identified have different nucleotide sequences in different individuals.

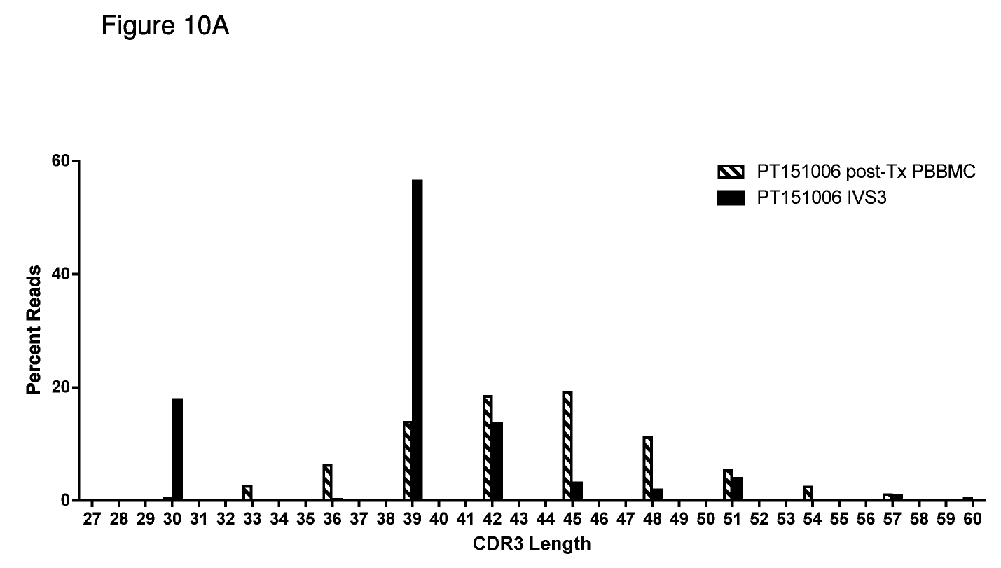

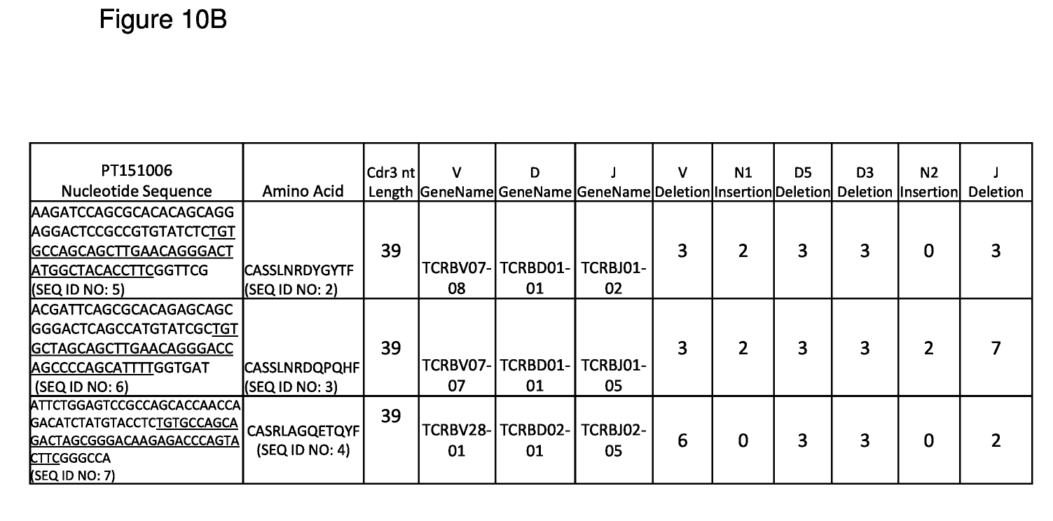

It has been reported that compared to private TCRs, shared TCR CDR3 aa sequences tended to be shorter on average by about one aa residue and, in addition, showed a significantly lower number of nucleotide (nt) insertions in the VD and DJ junction (Madi, et al, Genome Research, 2014). A comparison of TCRβ CDR3 length distribution in IVS3 showed that the T cells in IVS3 (FIG. 10A, black columns) have relatively shorter CDR3 region than the unstimulated T cells in post-Tx PBMC (FIG. 10A, hatched column). As shown in FIG. 10B, all three public TCR have the same CDR3 length (n=39 nucleotides). These TCRs also have relatively few nontemplated (NT) nucleotide (nc) addition at the junction region (0 to 2 of nucleotide additions). One of the public TCRs we identified, CASRLAGQETQYF (SEQ ID NO: 4), had no NT nc addition at the N1 (VD) insertion site and no nt addition at the N2 (DJ) insertion site. The phenotypic features of shorter length and limited number of NT nc additions support the conclusion that these TCRβ CDR3 sequences are public TCR sequences.

### Example 10: HLA Tissue Typing Results for Patients Treated with LV305

Patient samples were forwarded to a commercial HLA tissue typing service. The results are summarized in Table 4 below and confirm no overlap in HLA-A or HLA-B alleles between patients sharing the public TCR CDR3. The HLA-DR, DP or DQ alleles are also different between the patients.

TABLE 4
HLA Tissue Typing Results for 4 Patients Treated with LV305

| | | |
|---|---|---|
| PT151006 | | |
| HLA-A | *02:01 | *24:02 |
| HLA-B | *13:02P | *35:01 |
| HLA-C | *04:01 | *06:02 |
| HLA-DRB1 | *11:01 | *13:01 |
| HLA-DRB3 | *02:02 | — |
| HLA-DRB4 | — | — |
| HLA-DRB5 | — | — |
| HLA-DQB1 | *03:01 | *06:03P |
| HLA-DPB1 | *04:01 | — |
| HLA-DQA1 | *01:03/10 | *05:01:01G |
| HLA-DPA1 | *01:03P | — |
| PT151016 | | |
| HLA-A | *01:01 | *29:02 |
| HLA-B | *40:01 | *57:01 |
| HLA-C | *03:04 | *06:02 |
| HLA-DRB1 | *04:01 | *07:01P |
| HLA-DRB3 | — | — |
| HLA-DRB4 | *01:01 | *01:03 |
| HLA-DRB5 | — | — |
| HLA-DQB1 | *03:01 | *03:03 |
| HLA-DPB1 | *04:01 | *04:02 |
| HLA-DQA1 | *02:01 | *03:01:01G |
| HLA-DPA1 | *01:03P | — |
| PT151014 | | |
| HLA-A | *02:01 | *03:01 |
| HLA-B | *15:01 | *40:01 |
| HLA-C | *03:04 | — |
| HLA-DRB1 | *04:01 | *08:01 |
| HLA-DRB3 | — | — |
| HLA-DRB4 | *01:01 | — |
| HLA-DRB5 | — | — |
| HLA-DQB1 | *03:02 | *04:02 |
| HLA-DPB1 | *02:01P | *03:01:01G |
| HLA-DQA1 | *03:01:01G | *04:01:01G |
| HLA-DPA1 | *01:03P | — |
| PT151050 | | |
| HLA-A | *33:03 | *74:01 |
| HLA-B | *39:10 | *58:01 |
| HLA-C | *07:01 | *12:03 |
| HLA-DRB1 | *01:02P | *15:03P |
| HLA-DRB3 | — | — |
| HLA-DRB4 | — | — |
| HLA-DRB5 | *01:01P | — |
| HLA-DQB1 | *05:01 | *06:02 |
| HLA-DPB1 | No HLA-DPB type could | No HLA-DPB type could be |
| | be obtained for this sample. | obtained for this sample. The |
| | The probe reactivity from | probe reactivity |
| | rPCR-SSOP does not | from rPCR-SSOP |
| | correlate with any described | does not correlate with any |
| | HLA-DPB allele(s) | described HLA-DPB allele(s) |
| HLA-DQA1 | *01:01 | *01:02 |
| HLA-DPA1 | *01:03P | *02:01P |

### Example 11: Sequencing of the TCRA and TCRB Variable Regions of Public TCRS from Patients Treated with LV305

5′-RLM-RACE (Ambion, Austin, Tex.) was used for the TCR cloning. This method makes it feasible to identify a TCR without prior knowledge of the variable domain sequence. In brief, RNA was isolated from the NY-ESO-1 specific T cells using a Trizol 6 extraction. Purified RNA was then dephosphorylated, de-capped using Tobacco Acid Pyrophosphatase, and ligated to a 5′ adapter. Then, cDNA was prepared from the RNA using M-MLV reverse transcriptase (Ambion, Austin, Tex.) with random decamers following the manufacture's instruction. The cDNA was then used in the PCR. The PCR was performed with primers annealing to 5′ adapter and constant region of either TCRα or TCRβ (illustrative primers and sequences such as pCa1 and pCb1 are listed in the publication by Walchli, et al, (2011) A Practical Approach to T-Cell Receptor Cloning and Expression. PLoS ONE 6(11): e27930; but have been modified to add a restriction site for cloning purposes). The amplicons were then digested, gel purified, and cloned into a prepared vector using restriction sites incorporated into the PCR primers. Agar plates containing colonies were then sent out for sequencing of cloned TCR mRNA.

151 clones were sequenced from 2 separate rounds of PCR for TCRβ. Two different TCRβ variable region sequences were found to occur at high frequency, one of which contained the public Vβ CDR3 sequence previously identified and provided in SEQ ID NO: 4. The full-length TCRβ variable region sequence containing the public VβCDR3 of SEQ ID NO: 4 is provided in SEQ ID NO: 9 and is shown in FIG. 11. The other TCRβ variable region sequence is provided in SEQ ID NO: 16 and shown in FIG. 13 (the sequence may be annotated and the different regions of the TCR identified using standard methodologies such as those described at the IMGT database website). Almost 100 clones from 2 separate rounds of PCR were sequenced for TCR alpha. A single TCRα chain variable region was identified and is provided in SEQ ID NO: 8 and shown in FIG. 11. A BLAST search of the TCRα sequence indicates homology with a known NY-ESO-1 specific TCRα sequence (see PDB:2BNQ_D; Boulter et al., Protein Eng. (2003) 16 (9): 707-711; Chen, J. L. et al. (2000) J. Immunol., 165, 948-955). No known matches were identified in a similar search using the TCRβ sequence of SEQ ID NO: 9.

### Example 12: NY-ESO-1 Specific T Cells with Public TCRS Infiltrate into Tumor after Treatment with G100

This Example demonstrates that the public TCRβ CDR3 sequences that were originally identified from a sarcoma patient in the LV305 immunotherapy trial can be detected in patients from two different clinical trials using G100, intratumoral injection of glucopyranosyl lipid Adjuvant in stable emulsion (GLA-SE). The first G100 trial (NCT02035657) is a proof-of-concept trial of GLA-SE in patients with Merkel Cell Carcinoma (MCC). The second G100 trial (NCT02180698) is TLR4 agonist GLA-SE and radiation therapy in treating patients with soft tissue sarcoma that is metastatic or cannot be removed by surgery. Biopsies at the tumor site or a draining lymph node were taken before and after the administration of G100. DNA was extracted from the biopsy tissue and peripheral blood and deep sequencing of the TCRβ CDR3 region was carried out to evaluate the diversity of the T cell repertoire. Unexpectedly, several public TCRs identified from LV305 patient PT151006 were also detected in patients with Merkel Cell Carcinoma (MCC) and sarcoma, who received local immune modulation by intratumoral G100 treatment combined with irradiation. Table 5 to Table 7 lists the frequency of the 3 public TCRs in pre-G100 and post-G100 PBMC and biopsy samples in one MCC patient and two sarcoma patients. The first public TCRβ CDR3, CASSLNRDYGYTF (SEQ ID NO:2), was detectable in the MCC patient G2 and the sarcoma patient P13. The second public TCRβ CDR3, CASSLNRDQPQHF (SEQ ID NO:3), was detectable in the MCC patient G2 and the sarcoma patient P12. The third public TCRβ CDR3, CASRLAGQETQYF (SEQ ID NO:4), was detectable in MCC patient G2.

TABLE 5
Frequency of the 1^{st} Public TCRβ CDR3
Sequence, CASSLNRDYGYTF, in G100 patients

| | G100- | | G100- | | G100- | |
|---|---|---|---|---|---|---|
| | MCC-G2 | | Sarcoma-P12 | | Sarcoma-P13 | |
| Pa- | G2- | G2- | P12- | P12- | P13- | P13- |
| tient | PBMC | biopsy | PBMC | biopsy | PBMC | biopsy |
| Pre- | 0% | 0% | 0% | 0% | 0.000069% | 0% |
| G100 | | | | | | |
| Post- | 0% | 0.000442% | 0% | 0% | 0% | 0 |
| G100 | | | | | | |

Table 5: The frequency of the 1^{st} public TCRβ CDR3 sequence, CASSLNRDYGYTF (SEQ ID NO: 2), in G100 patients. Shown are the presence (frequency) of the public TCR in pre-G100 or post-G100 PBMC and tumor biopsy from a G100-MCC patient (G100-MCC-G2) and two G100-Sarcoma patients (G100-Sarcoma-P12 and G100-Sarcoma-P13). The sequence was detected at 0.000442% in post-Tx biopsy from patient G2 but not detectable in pre-Tx biopsy or PBMC samples.

TABLE 6
Frequency of the 2^{nd} Public TCRβ CDR3
Sequence, CASSLNRDQPQHF, in G100 patients

| | G100- | | G100- | | G100- | |
|---|---|---|---|---|---|---|
| | MCC-G2 | | Sarcoma-P12 | | Sarcoma-P13 | |
| Pa- | G2- | G2- | P12- | P12- | P13- | P13- |
| tient | PBMC | biopsy | PBMC | biopsy | PBMC | biopsy |
| Pre- | 0.000421% | 0% | 0.000154% | 0% | 0% | 0% |
| G100 | | | | | | |
| Post- | 0% | 0% | 0% | 0% | 0% | 0% |
| G100 | | | | | | |

Table 6: The frequency of the 2^{nd} public TCR TCRβ CDR3 sequence, CASSLNRDQPQHF, in G100 patients. Shown are the presence (frequency) of the public TCR in pre-G100 or post-G100 PBMC and tumor biopsy from a G100-MCC patient (G100-MCC-G2) and two G100-Sarcoma patients (G100-Sarcoma-P12 and G100-Sarcoma-P13). This sequence was detected in pre-Tx PBMC from G2 and P12.

TABLE 7
Frequency of the 3^{rd} Public TCRβ CDR3
Sequence, CASRLAGQETQYF, in G100 patients

| | G100- | | G100- | | G100- | |
|---|---|---|---|---|---|---|
| | MCC-G2 | | Sarcoma-P12 | | Sarcoma-P13 | |
| Pa- | G2- | G2- | P12- | P12- | P13- | P13- |
| tient | PBMC | biopsy | PBMC | biopsy | PBMC | biopsy |
| Pre- | 0% | 0% | 0% | 0% | 0% | 0% |
| G100 | | | | | | |
| Post- | 0% | 0.000442% | 0% | 0% | 0% | 0% |
| G100 | | | | | | |

Table 7: The frequency of the 3^{rd} public TCR TCRB CDR3 sequence, CASRLAGQETQYF, in G100 patients. Shown are the presence (frequency) of the public TCR in pre-G100 or post-G100 PBMC and tumor biopsy from a G100-MCC patient (G100-MCC-G2) and two G100-Sarcoma patients (G100-Sarcoma-P12 and G100-Sarcoma-P13). The sequence was detected at 0.000442% in post-Tx biopsy from patient G2 but not detectable in pre-Tx biopsy or PBMC samples.

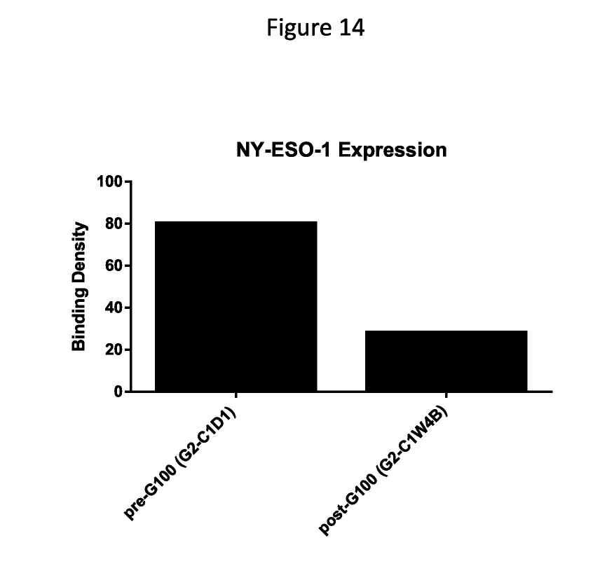

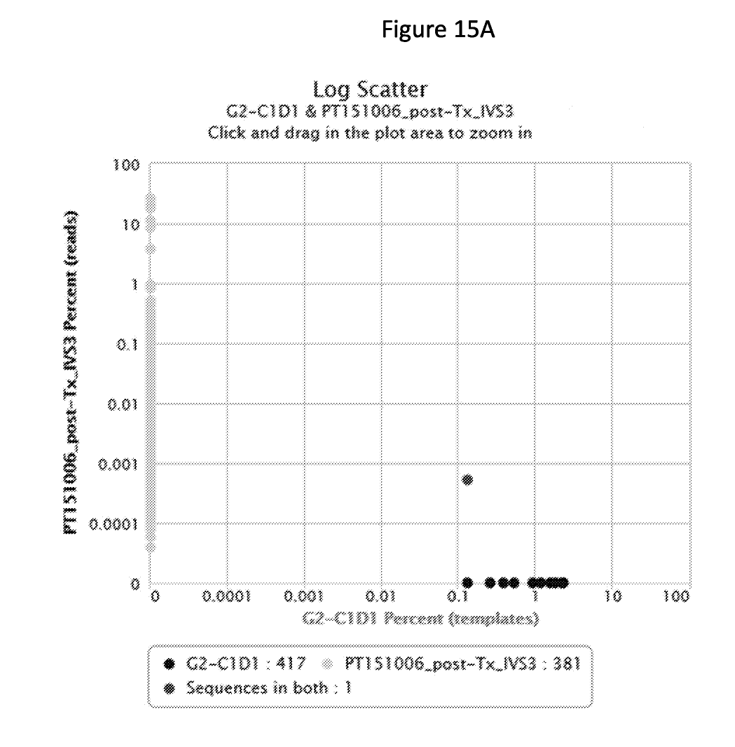

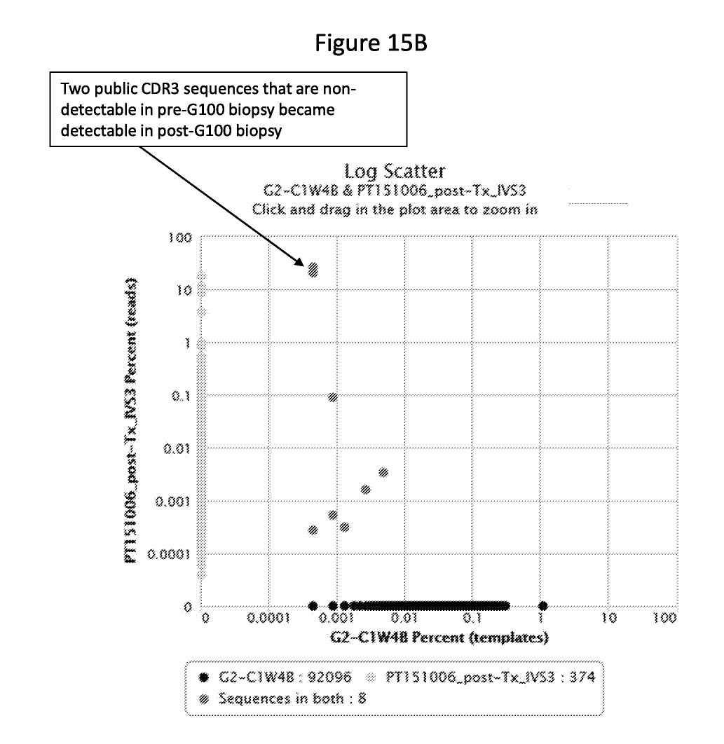

G2 is a MCC patient with a NY-ESO-1 expressing tumor who had a complete response following intratumoral G100 treatment. As shown in FIG. 14, the pre-G100 biopsy from this patient shows NY-ESO-1 expression, which was decreased by approximately 3 fold in the post-G100 biopsy, consistent with the disappearance of cytokeratin (CK20) positive tumor cells post-G100 treatment (data not shown). As shown in FIG. 15, two public TCRβ CDR3, which were non-detectable in pre-G100 biopsy, became detectable in post-G100 biopsy. These data suggest modulating the tumor microenvironment with G100 may have directed antigen-specific T cells with public TCRs into the tumor from the peripheral blood. These findings support further exploration of public TCRβ sequences as biomarkers for NY-ESO-1 specific immunotherapy.

### Example 13: The Public TCRB CDR3 Sequences Identified in Patients from LV305 Clinical Trial can Also be Detected in Patients from the C131 Clinical Trial

This example demonstrates that the three public TCRβ CDR3 sequences identified from PT151006, a patient from the LV305 trial (NCT02122861), can also be detected in patients from C131 (NCT02387125), a different clinical trial.

C131 is a Phase 1b safety study of CMB305 (sequentially administered LV305 and G305 (G305 consists of recombinant NY-ESO-1 protein formulated with a synthetic small molecule called glucopyranosyl lipid A (GLA), a TLR4 agonist, and is designed to boost the CTL response via the induction of antigen-specific CD4 “helper” T cells) in patients with locally advanced, relapsed, or metastatic cancer expressing NY-ESO-1. We examined the TCRβ CDR3 repertoire from 13 C131 patients using the public sequences identified from PT151006. As shown in Table 8, the first public TCRβ CDR3 sequence, CASSLNRDYGYTF (SEQ ID NO:2), was detected in 3 out of the 13 C131 patients; as shown in Table 9, the 2^{nd} public TCRβ CDR3 sequence, CASSLNRDQPQHF (SEQ ID NO:3), was detected in 6 out of 13 C131 patients; As shown in Table 10, the 3^{rd} public TCRβ CDR3 sequence, CASRLAGQETQYF (SEQ ID NO:4), was also detected in 6 out of 13 C131 patients. These data showed that the CDR3 sequences from PT151006 were shared in patients from different trials. In four patients these public TCR were detected after administration of the CMB305 regimen and are thus likely induced by the therapy.

TABLE 8
The 1st Public TCRβ CDR3 Sequence, CASSLNRDYGYTF, can be detected in 3 out of 13 C131 patients

| | C131- | C131- | C131- | C131- | C131- | C131- | C131- | C131- | C131- | C131- | C131- | C131- | C131- |
|---|---|---|---|---|---|---|---|---|---|---|---|---|---|
| | 001 | 002 | 003 | 007 | 009 | 010 | 011 | 012 | 013 | 017 | 018 | 020 | 026 |
| Pre-Tx | 6.71E−04% | 0% | 0% | 0% | 0% | 0% | 0% | 5.20E−04% | 0% | 0% | 0% | 0% | 0% |
| PBMC | | | | | | | | | | | | | |
| Post-Tx | 6.94E−04% | 0% | 0% | 0% | 0.001162% | 0% | 0% | 0% | 0% | 0% | 0% | 0% | 0% |
| PBMC | | | | | | | | | | | | | |
| IVS3 | 20% | | | | | | | | | | | | |
| from | | | | | | | | | | | | | |
| PT151006 | | | | | | | | | | | | | |

Table 8. The frequency of the 1^{st} public TCRβ CDR3 sequence in pre-Tx and post-Tx PBMC samples from 13 patients in the C131 trial. This table shows the frequency of the CDR3 sequence, CASSLNRDYGYTF (SEQ ID NO: 2), in 13 patients PBMC samples collected either before or after treatment with CMB305. The sequence can be detected in 3 out of 13 patients.

TABLE 9
The 2nd Public TCRβ CDR3 Sequence, CASSLNRDQPQHF,
can be detected in 6 out of 13 C131 patients

| | C131- | C131- | C131- | C131- | C131- | C131- | C131- |
|---|---|---|---|---|---|---|---|
| | 001 | 002 | 003 | 007 | 009 | 010 | 011 |
| Pre-Tx | 0% | 0% | 0% | 0% | 4.85E−04% | 0% | 0.001869% |
| PBMC | | | | | | | |
| Post-Tx | 0% | 0% | 0% | 0% | 0% | 0% | 0% |
| PBMC | | | | | | | |
| IVS3 | 8.5% | | | | | | |
| from | | | | | | | |
| PT151006 | | | | | | | |

| | C131- | C131- | C131- | C131- | C131- | C131- |
|---|---|---|---|---|---|---|
| | 012 | 013 | 017 | 018 | 020 | 026 |
| Pre-Tx | 2.85E−04% | 0.00133% | 0% | 5.00E−04% | 0% | 7.92E−04% |
| PBMC | | | | | | |
| Post-Tx | 0% | 0% | 0% | 0% | 0% | 0.001153% |
| PBMC | | | | | | |
| IVS3 | | | | | | |
| from | | | | | | |
| PT151006 | | | | | | |

Table 9. The frequency of the 2^{nd} public TCRβ CDR3 sequence in pre-Tx and post-Tx PBMC samples from 13 patients in the C131 trial. This table shows the frequency of the CDR3 sequence, CASSLNRDQPQHF (SEQ ID NO: 3), in 13 patients PBMC samples collected either before or after treatment with CMB305. The sequence can be detected in 6 out of 13 patients.

TABLE 10
The 3^{rd} Public TCRβ CDR3 Sequence, CASRLAGQETQYF,
can be detected in 6 out of 13 C131 patients

| | C131- | C131- | C131- | C131- | C131- | C131- | C131- |
|---|---|---|---|---|---|---|---|
| | 001 | 002 | 003 | 007 | 009 | 010 | 011 |
| Pre-Tx | 0.000336% | 0.000407% | 0% | 0% | 0.000485% | 0% | 0% |
| PBMC | | | | | | | |
| Post-Tx | 0% | 0% | 0% | 0% | 0% | 0% | 0% |
| PBMC | | | | | | | |
| IVS3 | 26% | | | | | | |
| from | | | | | | | |
| PT151006 | | | | | | | |

| | C131- | C131- | C131- | C131- | C131- | C131- |
|---|---|---|---|---|---|---|
| | 012 | 013 | 017 | 018 | 020 | 026 |
| Pre-Tx | 0% | 0.000771% | 0% | 0% | 0% | 0% |
| PBMC | | | | | | |
| Post-Tx | 0% | 0% | 0.000344% | 0.000429% | 0% | 0% |
| PBMC | | | | | | |
| IVS3 | | | | | | |
| from | | | | | | |
| PT151006 | | | | | | |

Table 10. The frequency of the 3^{rd} public TCRβ CDR3 sequence in pre-Tx and post-Tx PBMC samples from 13 patients in the C131 trial. This table shows the frequency of the CDR3 sequence, CASRLAGQETQYF (SEQ ID NO: 4), in 13 patients PBMC samples collected either before or after treatment with CMB305. The sequence can be detected in 6 out of 13 patients.

### Example 14: The CDR3 of the Three Public TCRβ Identified from LV305 Clinical Trial can be Detected in Patients from an Anti-CTLA-4 Trial

This example demonstrates that the public TCRβ CDR3 sequences identified from LV305 trial can be detected in patients receiving anti-CTLA4 mAb therapy with tremelimumab (Robert, et al, Clin Can Res, 2014).

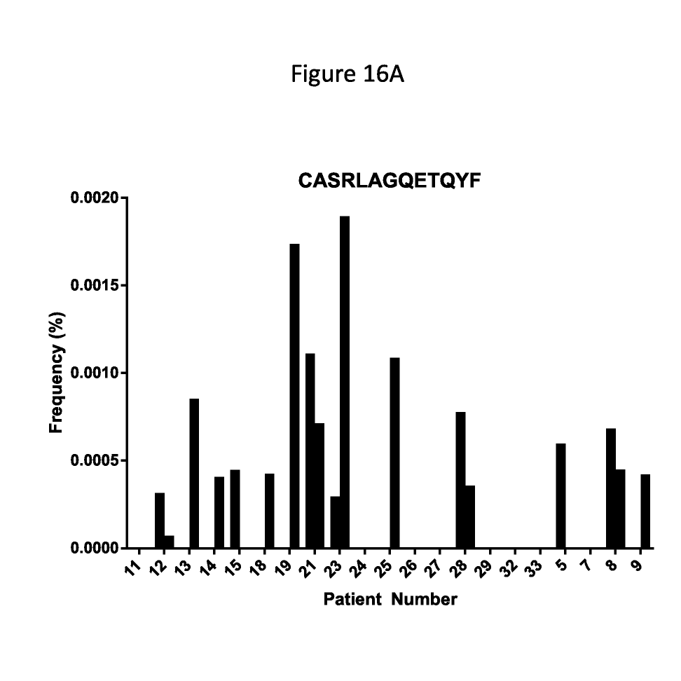

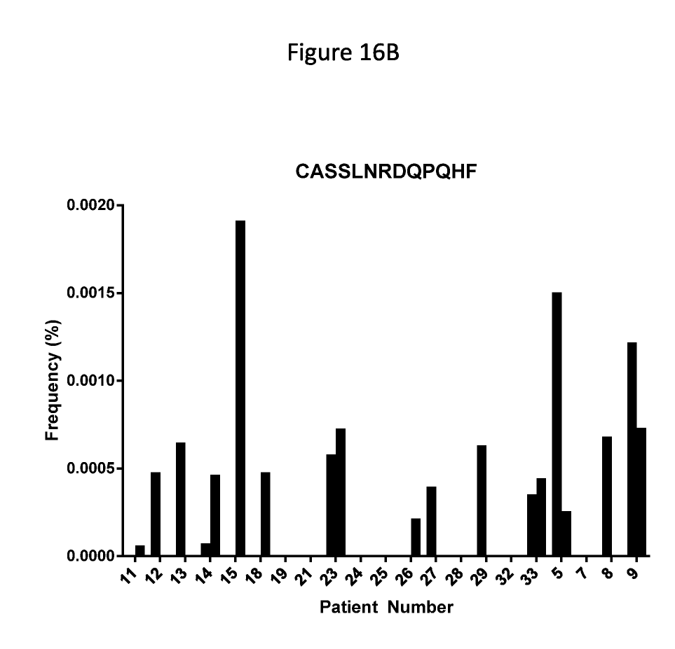

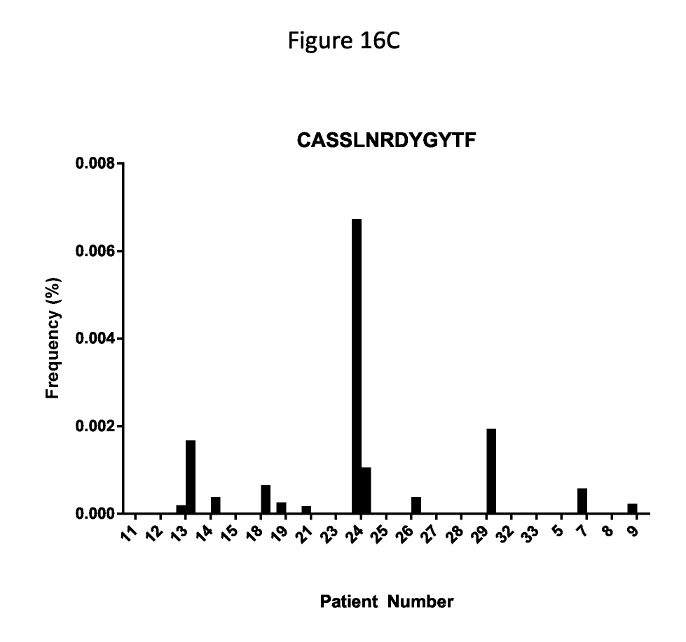

In this trial with tremelimumab, PBMC were collected at baseline and 30 to 60 days after receiving tremelimumab. Next-generation sequencing was used to study the CDR3 region of TCRβ. The sequencing data from 21 patients were deposited in the on-line database accessible through the Adaptive ImmunoSEQ Analyzer software. Database query comparing the CDR3 sequences from PT151006-IVS3 and the sequences of the 21 patients receiving anti-CTLA4 therapy showed that the 3 public sequences identified from PT151006 can also be detected in these patients on CTLA4 therapy (FIG. 16).

Increased TCR V-beta CDR3 richness and Shannon index diversity was observed in patients with CTLA4 blockade therapy (Robert, et al, Clin Can Res, 2014). Regarding the frequency of public TCR, there was no clear trend of whether the frequency increases or decreases after anti-CTLA4 therapy, and it varies from patient to patient. Of note, in one of the three patients that had completed response (CR) from the CTLA4 trial, GA18, all three public sequences were detected in post-Tx PBMC while none of them was detected in pre-Tx PBMC. The potential use of these public CDR3 sequences as biomarkers for treatment response needs to be further investigated.

### Example 15: Different TCRB V Usage for the Same CDR3 in the Same or Different Patients

This example demonstrates that the same CDR3 region can pair with different TCRβ V genes in different patients, or even in the same patient.

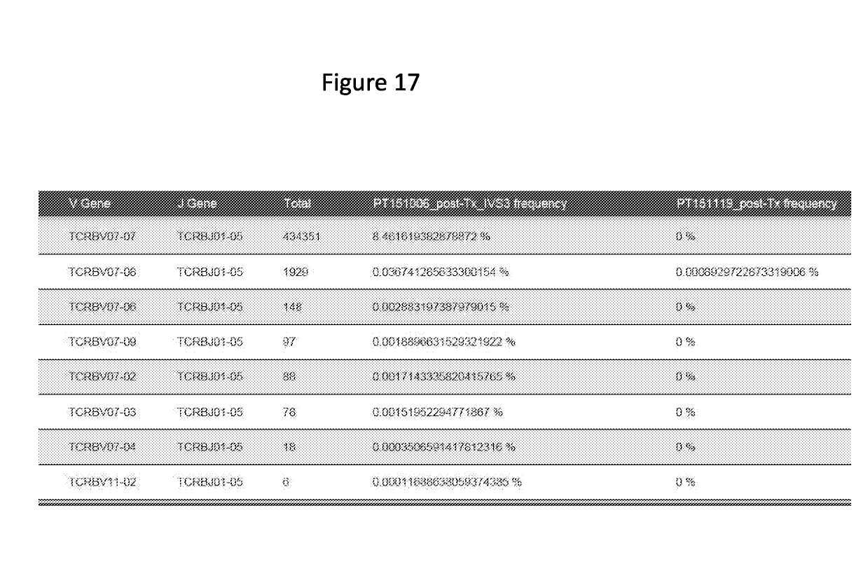

FIG. 17 lists the different TCRβ V gene usage that were detected to be associated with the same CDR3 by deep sequencing analysis. CASSLNRDQPQHF (SEQ ID NO:3) is the second public CDR3, which was detected in both PT151006 and PT151119. This CDR3 mainly uses TCRβ V07-07 in PT151006. It also uses TCRβ V07-08, TCRβ V07-06, TCRβ V07-09, TCRβ V07-02, TCRB V07-03, and TCRβ V07-04 in PT151006. In PT151119, only TCRβ V07-08 is used for this CDR3. This shows that different TCRβ V-gene families can be used by different patients for the same CDR3, and different TCRβ V-genes can also be used within the same patient for the same CDR3. Of note, the J gene usage (TCRβ J01-05) is the same for both patients.

FIG. 19 lists the nucleotide sequences, CDR3 amino acid sequences, and V gene and J gens of TCRβ in patients from different trials. PT151006 is from the LV305 clinical trial; C131-001 and C131-013 are from the CMB305 trial; G2-C1W4B is from the G100 trial. The data in this figure demonstrated that patients from different trials can have the same CDR3 amino acid sequences but with different nucleotide sequences and different TCRβ V-gene usage. In the case of patient C131-013, three different nucleotide sequences were found to encode the same CDR3, CASSLNRDQPQHF (SEQ ID NO:3), with different TCRβ V-gene usage. This is similar to the observation for PT151006, as shown in FIG. 17 and FIG. 18.

### Example 16: The Public CDR3 are Identified from NY-ESO-1 Specific CD4 T Cells

This example shows that the in vitro generated cell culture which was used to identify the public TCRβ CDR3 was composed of CD4 T cells.

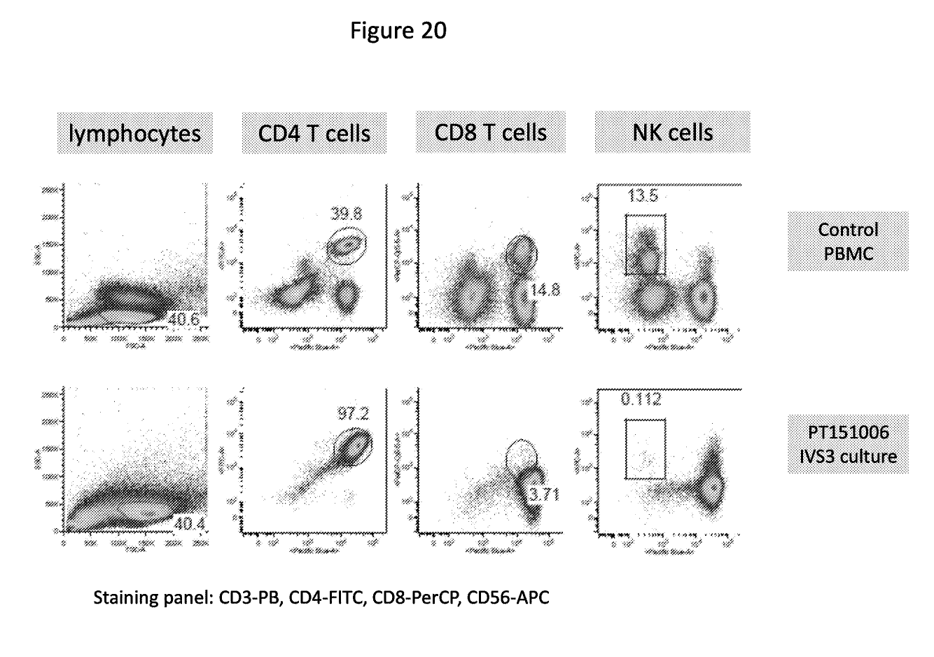

To characterize the phenotype of the PT151006-IVS3 T cell culture, we stained the cultured cells side-by-side with uncultured PBMC from a normal donor. The cells were stained with monoclonal antibodies against T cell markers (CD3, CD4, and CD8) and NK cell marker (CD56) using fluorochrome-conjugated monoclonal antibodies and then analyzed on a BD LSRII flow cytometer. Data analysis was done using the FlowJo software. As shown in FIG. 20, the lymphocytes population was first gated on the FSC/SSC plot, then CD4 T cells were gated as CD3⁺CD4⁺ lymphocytes and CD8 T cells were gated as CD3⁺CD8⁺ lymphocytes. The NK cells were gated as CD3⁻CD56⁺ lymphocytes. The control donor PBMC has the expected percentages of CD4, CD8 T cells and NK cells, as normally observed in healthy donor PBMC. In contrast, the cultured cells from PT151006-IVS3 show a lack of NK cells and CD8 T cells and only contains CD4 T cells. This data showed that the NY-ESO-1 specific T cell line that were cultured from PT151006 are CD4 T cells, and the public TCRs are CD4 TCRs.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A chimeric heterodimeric T cell receptor (TCR) polypeptide comprising:
a. a first polypeptide comprising a TCR beta chain variable region, a TCR beta chain constant region, and optionally a transmembrane domain and a cytoplasmic signaling domain;
b. a second polypeptide comprising a TCR alpha chain variable region, a TCR alpha chain constant region, and optionally a transmembrane domain and a cytoplasmic signaling domain;
wherein the heterodimeric TCR specifically binds to an NY-ESO-1/MHC complex, wherein the TCR beta chain variable region comprises the TCR beta chain variable region amino acid sequence set forth in SEQ ID NO:9;
wherein the TCR alpha chain variable region comprises the cognate TCR alpha chain variable region amino acid sequence set forth in SEQ ID NO:8; and wherein there is at least one disulfide bond between the first polypeptide and the second polypeptide.

2. The chimeric TCR of claim 1, wherein the TCR beta chain variable region CDR3 comprises the amino acid sequence CASRLAGQETQYF (SEQ ID NO: 4).

3. The chimeric heterodimeric TCR of claim 1 which is soluble, wherein the first polypeptide and the second polypeptide do not comprise the transmembrane domain and the cytoplasmic signaling domain.

4. A nucleic acid comprising a polynucleotide sequence that encodes the chimeric heterodimeric TCR of claim 1.

5. An expression vector comprising the nucleic acid of claim 4.

6. The expression vector of claim 5, that is a retroviral vector.

7. (canceled)

8. An isolated cell comprising the nucleic acid of claim 4 or the vector of claim 5.

9. The cell of claim 8, that is a T cell.

10. A pharmaceutical composition comprising the chimeric heterodimeric TCR of claim 1, the vector of claim 5, the nucleic acid of claim 4 or the isolated cell of claim 9.

11. A single chain TCR comprising a TCR beta chain variable region, a TCR alpha chain variable region, a constant region and optionally a transmembrane domain and a cytoplasmic signaling domain; wherein the TCR beta chain variable region CDR3 comprises an amino acid sequence selected from the group consisting of CASSLNRDYGYTF (SEQ ID NO: 2), CASSLNRDQPQHF (SEQ ID NO: 3) and CASRLAGQETQYF (SEQ ID NO: 4); wherein the single chain TCR is specific for an NY-ESO-1/MHC complex.

12. The single chain TCR of claim 11, wherein the TCR beta chain variable region comprises a TCR beta chain variable region amino acid sequence set forth in SEQ ID NO:9; wherein the TCR alpha chain variable region comprises the cognate TCR alpha chain variable region amino acid sequence as set forth in SEQ ID NO:8.

13. The single chain TCR of claim 11, that is a soluble single chain TCR; wherein the single chain TCR does not comprise the transmembrane domain and the cytoplasmic signaling domain.

14. A nucleic acid comprising a polynucleotide sequence that encodes the single chain TCR of claim 11.

15. An expression vector comprising the nucleic acid of claim 14.

16. The expression vector of claim 15, that is a retroviral vector.

17. (canceled)

18. An isolated cell comprising the nucleic acid of claim 14 or the vector of claim 16.

19. The cell of claim 18, that is a T cell.

20. A pharmaceutical composition comprising the single chain TCR of claim 11, the vector of claim 15, the nucleic acid of claim 15 or the cell of claim 20.

21. A method of treating an NY-ESO-1 cancer in a mammalian subject comprising administering to the subject a therapeutic composition, said composition comprising one or more therapeutic agents selected from the isolated cell of claim 9 and the isolated cell of claim 19; wherein the therapeutic composition is administered in an amount effective to treat the cancer in the subject.

22. A method of inhibiting proliferation of a cancer cell that expresses NY-ESO-1 in a mammalian subject comprising administering to the subject a therapy selected from the isolated cell of claim 9 or the isolated cell of claim 19; wherein the therapeutic composition is administered in an amount effective to inhibit proliferation of the cancer cell that expresses NY-ESO-1 in the subject.

23. A method of treating cancer comprising:
(a) identifying a mammalian subject as likely to benefit from a NY-ESO-1 cancer therapy comprising determining in a sample from the mammalian subject the presence of
(i) a polynucleotide encoding a TCR polypeptide comprising a TCR beta chain variable region complementarity determining region 3 (VβCDR3) that is specific for NY-ESO-1, wherein the Vβ CDR3 comprises the amino acid sequence of CASSLNRDXXXXF (SEQ ID NO: 1); or CASRLAGQETQYF (SEQ ID NO: 4); or
both; or
(ii) a TCR polypeptide comprising a VβCDR3 that is specific for NY-ESO-1, wherein the VβCDR3 comprises the amino acid sequence of CASSLNRDXXXXF (SEQ ID NO: 1); or CASRLAGQETQYF (SEQ ID NO: 4), or both;
wherein the presence of (i) and/or (ii) is indicative that the subject is likely to benefit from the NY-ESO-1 cancer therapy; and
(b) administering the NY-ESO-1 cancer therapy to the mammalian subject.

24. The method of claim 23, wherein the VβCDR3 comprises an amino acid sequence selected from the group consisting of CASSLNRDYGYTF (SEQ ID NO: 2), CASSLNRDQPQHF (SEQ ID NO: 3) and CASRLAGQETQYF (SEQ ID NO: 4), or any combination of one or more of the foregoing.

25.-30. (canceled)

31. The method of claim 23, wherein the NY-ESO-1 cancer therapy comprises administering to the subject a composition comprising GLA, said composition comprising:
(a) GLA of the formula: 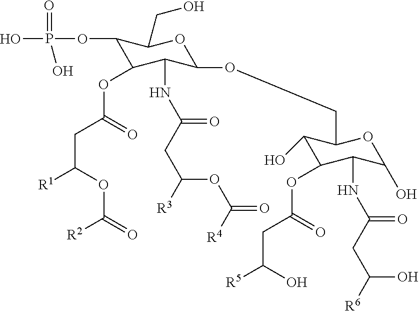
wherein:
R¹, R³, R⁵ and R⁶ are C₁₁-C₂₀ alkyl; and
R² and R⁴ are C₁₂-C₂₀ alkyl; and
(b) a pharmaceutically acceptable carrier or excipient;
wherein the composition does not comprise antigen.

32. The method of claim 31, wherein R¹, R³, R⁵ and R⁶ are undecyl and R² and R⁴ are tridecyl.

33.-37. (canceled)

38. The method of claim 31, wherein the composition is administered by subcutaneous, intradermal, intramuscular, intratumoral, or intravenous injection.

39. The method of any of claims 21-23, wherein the composition is administered in conjunction with one or more additional therapeutic agents or treatments.

40. The method of claim 39, wherein the therapeutic agent is an immune checkpoint inhibitor.

41.-44. (canceled)

45. The method of claim 39, wherein the one or more additional therapeutic treatments is radiation therapy.

46. A method of identifying a mammalian subject that is likely to benefit from an NY-ESO-1 cancer therapy comprising:
(a) determining in a sample from the mammalian subject the presence of
(i) a polynucleotide encoding a TCR polypeptide comprising a VβCDR3 that is specific for NY-ESO-1, wherein the VβCDR3 comprises the amino acid sequence of CASSLNRDXXXXF (SEQ ID NO: 1) or CASRLAGQETQYF (SEQ ID NO: 4); or both; or
(ii) a TCR polypeptide comprising a VβCDR3 that is specific for NY-ESO-1, wherein the VβCDR3 comprises the amino acid sequence of CASSLNRDXXXXF (SEQ ID NO: 1) or CASRLAGQETQYF (SEQ ID NO: 4); or
both;
wherein the presence of (i) and/or (ii) is indicative that the subject is likely to benefit from the NY-ESO-1 cancer therapy.

47. The method of claim 46, wherein the VβCDR3 comprises an amino acid sequence selected from the group consisting of CASSLNRDYGYTF (SEQ ID NO: 2), CASSLNRDQPQHF (SEQ ID NO: 3) and CASRLAGQETQYF (SEQ ID NO: 4), or any combination of one or more of the foregoing.

48. A method for detecting cells or tissue comprising an NY-ESO-1 peptide antigen presented on the cells or tissue in the context of an MHC complex, the method comprising: a) contacting the cells or tissue with at least one soluble TCR molecule or functional fragment thereof of claim 3 or claim 13 under conditions that form a specific binding complex between the presented NY-ESO-1 peptide antigen and the soluble TCR or fragment, b) washing the cells or tissue under conditions appropriate to remove any soluble TCR molecule or fragment not bound to the presented peptide antigen; and c) detecting the specific binding complex as being indicative of cells or tissue comprising the presented peptide antigen.


SEQUENCE LISTING (26 sequences)

SEQ ID NO: 1 - protein, 13 aa, Artificial Sequence
  Public TCR CDR3 consensus sequence
  MISC_FEATURE (9)..(12): Xaa is any amino acid
CASSLNRDXXXXF

SEQ ID NO: 2 - protein, 13 aa, Homo sapiens
CASSLNRDYGYTF

SEQ ID NO: 3 - protein, 13 aa, Homo sapiens
CASSLNRDQPQHF

SEQ ID NO: 4 - protein, 13 aa, Homo sapiens
CASRLAGQETQYF

SEQ ID NO: 5 - DNA, 87 bp, Homo sapiens
aagatccagcgcacacagcaggaggactccgccgtgtatctctgtgccagcagcttgaacagggactatggctacaccttcggttcg

SEQ ID NO: 6 - DNA, 87 bp, Homo sapiens
acgattcagcgcacagagcagcgggactcagccatgtatcgctgtgctagcagcttgaacagggaccagccccagcattttggtgat

SEQ ID NO: 7 - DNA, 87 bp, Homo sapiens
attctggagtccgccagcaccaaccagacatctatgtacctctgtgccagcagactagcgggacaagagacccagtacttcgggcca

SEQ ID NO: 8 - protein, 200 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVVGGSYIPTFGRGTSLIVHPYIQKPDPAVYQLRDSKSSDKSVCLFTDFDSQTNVSQSKDSDVYITDKTVLDMRSMDFKSNSAVAWSNKSD

SEQ ID NO: 9 - protein, 117 aa, Homo sapiens
DVKVTQSSRYLVKRTGEKVFLECVQDMDHENMFWYRQDPGLGLRLIYFSYDVKMKEKGDIPEGYSVSREKKERFSLILESASTNQTSMYLCASRLAGQETQYFGPGTRLLVLEDLKN

SEQ ID NO: 10 - DNA, 87 bp, Homo sapiens
aagatccagccctcagaacccagggactcagctgtgtacttctgtgccagcagtttgaaccgggactatggctacaccttcggttcg

SEQ ID NO: 11 - DNA, 87 bp, Homo sapiens
aagatccagcgcacagagcggggggactcagccgtgtatctctgtgccagcagcttaaacagggactatggctacaccttcggttcg

SEQ ID NO: 12 - DNA, 87 bp, Homo sapiens
gagatccagcgcacagagcagggggactcggccatgtatctctgtgccagcagcttaaaccgggaccagccccagcattttggtgat

SEQ ID NO: 13 - DNA, 87 bp, Homo sapiens
aatgtgaacgccttggagctggacgactcggccctgtatctctgtgccagcagcttgaatcgtgatcagccccagcattttggtgat

SEQ ID NO: 14 - DNA, 87 bp, Homo sapiens
aggctggagttggctgctccctcccagacatctgtgtacttctgtgccagcagactagcggggcaagagacccagtacttcgggcca

SEQ ID NO: 15 - DNA, 87 bp, Homo sapiens
aatgtgagcaccttggagctgggggactcggccctttatctttgcgccagcagactagcggggcaagagacccagtacttcgggcca

SEQ ID NO: 16 - protein, 170 aa, Homo sapiens
MEAVVTTLPREGGVRPSRKMLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARKGLAGRETQYFGPGTRLLVLEDLKNVFPPEVAVFEPSEAE

SEQ ID NO: 17 - DNA, 87 bp, Homo sapiens
aagatccggtccacaaagctggaggactcagccatgtacttctgtgccagcagtctaaacagggactatggctacaccttcggttcg

SEQ ID NO: 18 - DNA, 87 bp, Homo sapiens
attctggagtccgccagcaccaaccagacatctatgtacctctgtgccagcagtttaaaccgggattatggctacaccttcggttcg

SEQ ID NO: 19 - DNA, 87 bp, Homo sapiens
aatgtgaacgccttggagctggaggactcggccctgtatctctgtgccagcagcttgaacagggatcagccccagcattttggtgat

SEQ ID NO: 20 - DNA, 87 bp, Homo sapiens
aacatgagctccttggagctgggggactcagccctgtacttctgtgccagcagcttgaacagggatcagccccagcattttggtgat

SEQ ID NO: 21 - DNA, 87 bp, Homo sapiens
aatgtgaacgccttgttgctgggggactcggccctgtatctctgtgccagcagcttgaacagggatcagccccagcattttggtgat

SEQ ID NO: 22 - DNA, 87 bp, Homo sapiens
aggctggagttggctgctccctcccagacatctgtgtacttctgtgccagcagacttgcgggccaagagacccagtacttcgggcca

SEQ ID NO: 23 - DNA, 87 bp, Homo sapiens
accctggagtctgccaggccctcacatacctctcagtacctctgtgccagcagactagcgggacaggagacccagtacttcgggcca

SEQ ID NO: 24 - DNA, 39 bp, Homo sapiens
tgtgctagcagcttgaacagggaccagccccagcatttt

SEQ ID NO: 25 - DNA, 39 bp, Homo sapiens
tgtgccagcagcttaaacagggaccagccccagcatttt

SEQ ID NO: 26 - DNA, 39 bp, Homo sapiens; identical to SEQ ID NO: 25